SEQUENCE LISTING (94 sequences)

SEQ ID NO: 1 - DNA, 22 bp, Artificial Sequence
  Description of Artificial Sequence Synthetic primer
tgaggtagtaggttgtatagtt

SEQ ID NO: 2 - DNA, 22 bp, Artificial Sequence
  Description of Artificial Sequence Synthetic primer
tgaggtagtagattgtatagtt

SEQ ID NO: 3 - DNA, 21 bp, Artificial Sequence
  Description of Artificial Sequence Synthetic primer
tcgtaccgtgagtaataatgc

SEQ ID NO: 4 - DNA, 22 bp, Artificial Sequence
  Description of Artificial Sequence Synthetic primer
tagcagcacgtaaatattggcg

SEQ ID NO: 5 - DNA, 23 bp, Artificial Sequence
  Description of Artificial Sequence Synthetic primer
tgtgcaaatccatgcaaaactga

SEQ ID NO: 6 - DNA, 21 bp, Artificial Sequence
  Description of Artificial Sequence Synthetic primer
ctgacctatgaattgacagcc

SEQ ID NO: 7 - DNA, 21 bp, Artificial Sequence
  Description of Artificial Sequence Synthetic primer
tagcagcacagaaatattggc

SEQ ID NO: 8 - DNA, 22 bp, Artificial Sequence
  Description of Artificial Sequence Synthetic primer
tacagtagtctgcacattggtt

SEQ ID NO: 9 - DNA, 22 bp, Artificial Sequence
  Description of Artificial Sequence Synthetic primer
tagcttatcagactgatgttga

SEQ ID NO: 10 - DNA, 22 bp, Artificial Sequence
  Description of Artificial Sequence Synthetic primer
gtgaaatgtttaggaccactag

SEQ ID NO: 11 - DNA, 21 bp, Artificial Sequence
  Description of Artificial Sequence Synthetic primer
atgacctatgaattgacagac

SEQ ID NO: 12 - DNA, 21 bp, Artificial Sequence
  Description of Artificial Sequence Synthetic primer
atcacattgccagggatttcc

SEQ ID NO: 13 - DNA, 21 bp, Artificial Sequence
  Description of Artificial Sequence Synthetic primer
atcacattgccagggattacc

SEQ ID NO: 14 - DNA, 22 bp, Artificial Sequence
  Description of Artificial Sequence Synthetic primer
tggctcagttcagcaggaacag

SEQ ID NO: 15 - DNA, 21 bp, Artificial Sequence
  Description of Artificial Sequence Synthetic primer
ttcaagtaatccaggataggc

SEQ ID NO: 16 - DNA, 21 bp, Artificial Sequence
  Description of Artificial Sequence Synthetic primer
tagcaccatctgaaatcggtt

SEQ ID NO: 17 - DNA, 23 bp, Artificial Sequence
  Description of Artificial Sequence Synthetic primer
aaaagctgggttgagagggcgaa

SEQ ID NO: 18 - DNA, 22 bp, Artificial Sequence
  Description of Artificial Sequence Synthetic primer
tttgttcgttcggctcgcgtga

SEQ ID NO: 19 - DNA, 22 bp, Artificial Sequence
  Description of Artificial Sequence Synthetic primer
ctggacttggagtcagaaggcc

SEQ ID NO: 20 - DNA, 22 bp, Artificial Sequence
  Description of Artificial Sequence Synthetic primer
gttctcccaacgtaagcccagc

SEQ ID NO: 21 - DNA, 22 bp, Artificial Sequence
  Description of Artificial Sequence Synthetic primer
taacactgtctggtaaagatgg

SEQ ID NO: 22 - DNA, 21 bp, Artificial Sequence
  Description of Artificial Sequence Synthetic primer
ttcacagtggctaagttctgc

SEQ ID NO: 23 - DNA, 22 bp, Artificial Sequence
  Description of Artificial Sequence Synthetic primer
cacacactgcaattacttttgc

SEQ ID NO: 24 - DNA, 22 bp, Artificial Sequence
  Description of Artificial Sequence Synthetic primer
catgccttgagtgtaggaccgt

SEQ ID NO: 25 - DNA, 24 bp, Artificial Sequence
  Description of Artificial Sequence Synthetic primer
tactgcatcaggaactgattggat

SEQ ID NO: 26 - DNA, 22 bp, Artificial Sequence
  Description of Artificial Sequence Synthetic primer
taacactgtctggtaacgatgt

SEQ ID NO: 27 - DNA, 22 bp, Artificial Sequence
  Description of Artificial Sequence Synthetic primer
tgagacctctgggttctgagct

SEQ ID NO: 28 - DNA, 17 bp, Artificial Sequence
  Description of Artificial Sequence Synthetic primer
ctcgcttcggcagcaca

SEQ ID NO: 29 - DNA, 19 bp, Artificial Sequence
  Description of Artificial Sequence Synthetic primer
gtctcctctgacttcaaca

SEQ ID NO: 30 - DNA, 20 bp, Artificial Sequence
  Description of Artificial Sequence Synthetic primer
caggaaatgagcttgacaaa

SEQ ID NO: 31 - DNA, 20 bp, Artificial Sequence
  Description of Artificial Sequence Synthetic primer
ctcaagaatgggcagaaagc

SEQ ID NO: 32 - DNA, 20 bp, Artificial Sequence
  Description of Artificial Sequence Synthetic primer
cttcaggaacagccaccaat

SEQ ID NO: 33 - DNA, 19 bp, Artificial Sequence
  Description of Artificial Sequence Synthetic primer
ccaaagtgtgaacgtgaag

SEQ ID NO: 34 - DNA, 18 bp, Artificial Sequence
  Description of Artificial Sequence Synthetic primer
tgggggatgcaggattga

SEQ ID NO: 35 - DNA, 22 bp, Artificial Sequence
  Description of Artificial Sequence Synthetic primer
tctacttgcacactctcccatt

SEQ ID NO: 36 - DNA, 20 bp, Artificial Sequence
  Description of Artificial Sequence Synthetic primer
gcctctatcacagtggctga

SEQ ID NO: 37 - RNA, 22 nt, Artificial Sequence
  Description of Artificial Sequence Synthetic oligonucleotide
ugagguaguagguuguauaguu

SEQ ID NO: 38 - RNA, 22 nt, Artificial Sequence
  Description of Artificial Sequence Synthetic oligonucleotide
ugagguaguagguugugugguu

SEQ ID NO: 39 - RNA, 22 nt, Artificial Sequence
  Description of Artificial Sequence Synthetic oligonucleotide
ugagguaguagauuguauaguu

SEQ ID NO: 40 - RNA, 22 nt, Artificial Sequence
  Description of Artificial Sequence Synthetic oligonucleotide
ugagguaguaguuugugcuguu

SEQ ID NO: 41 - RNA, 23 nt, Artificial Sequence
  Description of Artificial Sequence Synthetic oligonucleotide
aaaagugcuuacagugcagguag

SEQ ID NO: 42 - RNA, 23 nt, Artificial Sequence
  Description of Artificial Sequence Synthetic oligonucleotide
agcagcauuguacagggcuauca

SEQ ID NO: 43 - RNA, 23 nt, Artificial Sequence
  Description of Artificial Sequence Synthetic oligonucleotide
ccugcagcgacuugauggcuucc

SEQ ID NO: 44 - RNA, 22 nt, Artificial Sequence
  Description of Artificial Sequence Synthetic oligonucleotide
acggguuaggcucuugggagcu

SEQ ID NO: 45 - RNA, 22 nt, Artificial Sequence
  Description of Artificial Sequence Synthetic oligonucleotide
ucguaccgugaguaauaaugcg

SEQ ID NO: 46 - RNA, 17 nt, Artificial Sequence
  Description of Artificial Sequence Synthetic oligonucleotide
gugggggagaggcuguc

SEQ ID NO: 47 - RNA, 22 nt, Artificial Sequence
  Description of Artificial Sequence Synthetic oligonucleotide
uucuggaauucugugugaggga

SEQ ID NO: 48 - RNA, 22 nt, Artificial Sequence
  Description of Artificial Sequence Synthetic oligonucleotide
uaacacugucugguaaagaugg

SEQ ID NO: 49 - RNA, 22 nt, Artificial Sequence
  Description of Artificial Sequence Synthetic oligonucleotide
ucucccaacccuuguaccagug

SEQ ID NO: 50 - RNA, 22 nt, Artificial Sequence
  Description of Artificial Sequence Synthetic oligonucleotide
uagcagcacguaaauauuggcg

SEQ ID NO: 51 - RNA, 23 nt, Artificial Sequence
  Description of Artificial Sequence Synthetic oligonucleotide
caacggaaucccaaaagcagcug

SEQ ID NO: 52 - RNA, 21 nt, Artificial Sequence
  Description of Artificial Sequence Synthetic oligonucleotide
cugaccuaugaauugacagcc

SEQ ID NO: 53 - RNA, 22 nt, Artificial Sequence
  Description of Artificial Sequence Synthetic oligonucleotide
aacuggcccucaaagucccgcu

SEQ ID NO: 54 - RNA, 21 nt, Artificial Sequence
  Description of Artificial Sequence Synthetic oligonucleotide
uagcagcacagaaauauuggc

SEQ ID NO: 55 - RNA, 23 nt, Artificial Sequence
  Description of Artificial Sequence Synthetic oligonucleotide
cccaguguucagacuaccuguuc

SEQ ID NO: 56 - RNA, 23 nt, Artificial Sequence
  Description of Artificial Sequence Synthetic oligonucleotide
ugugcaaauccaugcaaaacuga

SEQ ID NO: 57 - RNA, 22 nt, Artificial Sequence
  Description of Artificial Sequence Synthetic oligonucleotide
uaacacugucugguaacgaugu

SEQ ID NO: 58 - RNA, 22 nt, Artificial Sequence
  Description of Artificial Sequence Synthetic oligonucleotide
uaauacugccugguaaugauga

SEQ ID NO: 59 - RNA, 23 nt, Artificial Sequence
  Description of Artificial Sequence Synthetic oligonucleotide
uaauacugccggguaaugaugga

SEQ ID NO: 60 - RNA, 22 nt, Artificial Sequence
  Description of Artificial Sequence Synthetic oligonucleotide
gugaaauguuuaggaccacuag

SEQ ID NO: 61 - RNA, 23 nt, Artificial Sequence
  Description of Artificial Sequence Synthetic oligonucleotide
uaaagugcuuauagugcagguag

SEQ ID NO: 62 - RNA, 22 nt, Artificial Sequence
  Description of Artificial Sequence Synthetic oligonucleotide
uagcuuaucagacugauguuga

SEQ ID NO: 63 - RNA, 21 nt, Artificial Sequence
  Description of Artificial Sequence Synthetic oligonucleotide
augaccuaugaauugacagac

SEQ ID NO: 64 - RNA, 23 nt, Artificial Sequence
  Description of Artificial Sequence Synthetic oligonucleotide
uacugcaucaggaacugauugga

SEQ ID NO: 65 - RNA, 22 nt, Artificial Sequence
  Description of Artificial Sequence Synthetic oligonucleotide
ugucaguuugucaaauacccca

SEQ ID NO: 66 - RNA, 21 nt, Artificial Sequence
  Description of Artificial Sequence Synthetic oligonucleotide
aucacauugccagggauuucc

SEQ ID NO: 67 - RNA, 21 nt, Artificial Sequence
  Description of Artificial Sequence Synthetic oligonucleotide
aucacauugccagggauuacc

SEQ ID NO: 68 - RNA, 22 nt, Artificial Sequence
  Description of Artificial Sequence Synthetic oligonucleotide
uggcucaguucagcaggaacag

SEQ ID NO: 69 - RNA, 22 nt, Artificial Sequence
  Description of Artificial Sequence Synthetic oligonucleotide
uucaaguaauccaggauaggcu

SEQ ID NO: 70 - RNA, 21 nt, Artificial Sequence
  Description of Artificial Sequence Synthetic oligonucleotide
uucacaguggcuaaguucugc

SEQ ID NO: 71 - RNA, 22 nt, Artificial Sequence
  Description of Artificial Sequence Synthetic oligonucleotide
uagcaccaucugaaaucgguua

SEQ ID NO: 72 - RNA, 22 nt, Artificial Sequence
  Description of Artificial Sequence Synthetic oligonucleotide
uauacaagggcagacucucucu

SEQ ID NO: 73 - RNA, 22 nt, Artificial Sequence
  Description of Artificial Sequence Synthetic oligonucleotide
caauuuagugugugugauauuu

SEQ ID NO: 74 - RNA, 22 nt, Artificial Sequence
  Description of Artificial Sequence Synthetic oligonucleotide
aaaagcuggguugagagggcga

SEQ ID NO: 75 - RNA, 22 nt, Artificial Sequence
  Description of Artificial Sequence Synthetic oligonucleotide
uuuguucguucggcucgcguga

SEQ ID NO: 76 - RNA, 21 nt, Artificial Sequence
  Description of Artificial Sequence Synthetic oligonucleotide
acuggacuuggagucagaagg

SEQ ID NO: 77 - RNA, 23 nt, Artificial Sequence
  Description of Artificial Sequence Synthetic oligonucleotide
uucucgaggaaagaagcacuuuc

SEQ ID NO: 78 - RNA, 22 nt, Artificial Sequence
  Description of Artificial Sequence Synthetic oligonucleotide
caugccuugaguguaggaccgu

SEQ ID NO: 79 - RNA, 23 nt, Artificial Sequence
  Description of Artificial Sequence Synthetic oligonucleotide
ugagugugugugugugagugugu

SEQ ID NO: 80 - RNA, 21 nt, Artificial Sequence
  Description of Artificial Sequence Synthetic oligonucleotide
caaagaggaaggucccauuac

SEQ ID NO: 81 - RNA, 22 nt, Artificial Sequence
  Description of Artificial Sequence Synthetic oligonucleotide
cacacacugcaauuacuuuugc

SEQ ID NO: 82 - RNA, 21 nt, Artificial Sequence
  Description of Artificial Sequence Synthetic oligonucleotide
ucuaguaagaguggcagucga

SEQ ID NO: 83 - RNA, 21 nt, Artificial Sequence
  Description of Artificial Sequence Synthetic oligonucleotide
uggguuuacguugggagaacu

SEQ ID NO: 84 - RNA, 21 nt, Artificial Sequence
  Description of Artificial Sequence Synthetic oligonucleotide
uggaggagaaggaaggugaug

SEQ ID NO: 85 - RNA, 22 nt, Artificial Sequence
  Description of Artificial Sequence Synthetic oligonucleotide
ugagaccucuggguucugagcu

SEQ ID NO: 86 - RNA, 24 nt, Artificial Sequence
  Description of Artificial Sequence Synthetic oligonucleotide
uggggagcugaggcucugggggug

SEQ ID NO: 87 - DNA, 15 bp, Homo sapiens
ttgttatttaggtca

SEQ ID NO: 88 - DNA, 19 bp, Homo sapiens
cacatgtcagccactgtga

SEQ ID NO: 89 - DNA, 21 bp, Homo sapiens
gaaatgatttcacagtgtgtg

SEQ ID NO: 90 - DNA, 18 bp, Homo sapiens
gtctttcttgtaaggcat

SEQ ID NO: 91 - DNA, 22 bp, Homo sapiens
gtttaatgttaattatgcagtg

SEQ ID NO: 92 - DNA, 21 bp, Homo sapiens
taatgttaattatgcagtgtt

SEQ ID NO: 93 - DNA, 19 bp, Homo sapiens
atgttaattatgcagtgtt

SEQ ID NO: 94 - DNA, 19 bp, Homo sapiens
ttagagcagagaggtttcg

(19) United States
(12) Patent Application Publication
(10) Pub. No.: US 2011/0117111 A1
(43) Pub. Date: May 19, 2011

(54) MICRORNA-BASED DIAGNOSTIC TESTING AND THERAPIES FOR INFLAMMATORY BOWEL DISEASE AND RELATED DISEASES

(75) Inventors: John H. Kwon, Lutherville, MD (US); Feng Wu, Lutherville, MD (US)

(73) Assignee: JOHNS HOPKINS UNIVERSITY, Baltimore, MD (US)

(21) Appl. No.: 12/934,820

(22) PCT Filed: Mar. 26, 2009
(86) PCT No.: PCT/US2009/038424

§ 371 Date: Jan. 13, 2011

Related U.S. Application Data
(60) Provisional application No. 61/070,829, filed on Mar. 26, 2008.

(51) Int. Cl. C12Q 1/68; C40B 30/04; A61K 31/7088; A61K 31/7105; A61K 38/00; A61K 39/395; G01N 33/53; G01N 33/566; A61P 1/00; A61P 29/00

(52) U.S. Cl. 424/172.1; 435/6; 506/9; 514/44 A; 514/44 R; 514/1.1; 435/7.1; 436/501

(57) ABSTRACT

The present invention is based, at least in part, on the novel discovery that certain microRNAs are associated with inflammatory bowel diseases and other related diseases. Accordingly, the invention relates to microRNA-based compositions, kits, and methods for detecting, characterizing, modulating, preventing, and treating inflammatory bowel diseases and other related diseases.

### RELATED APPLICATIONS

This application claims the benefit of U.S. Provisional Application Ser. No. 61/070,829, filed on Mar. 26, 2008; the entire contents of the application is incorporated herein by reference.

### GOVERNMENT FUNDING

Work described herein was supported, at least in part, by the National Institutes of Health (NIH) under grants K08DK078046 and R24DK064388. The government may therefore have certain rights to this invention.

### BACKGROUND OF THE INVENTION

Inflammatory bowel diseases (also referred to herein as “IBD”) comprise a group of conditions characterized by chronic relapsing inflammation affecting the gastrointestinal tract, including both the small and large intestine. These conditions often share similar clinical characteristics that make specific distinction difficult. In particular, acute and chronic inflammation of the colon may be seen in both diseases. Studies examining the global gene expression profiles in IBD demonstrate the increased expression of numerous genes involved in inflammation and fibrosis. Most therapies for both diseases have been aimed at decreasing the global inflammation through the use of corticosteroids, immune modulators and other biologic therapies. However, current therapies remain inadequate because the precise mechanisms of pathology remain unknown. In particular, satisfactory treatment of IBD is an unmet medical need, as existing therapeutic agents have not been successful in curtailing the disease and avoiding the need for surgery. Up to 40% of all ulcerative colitis patients undergo surgery, which typically includes either the removal of part of the large intestine or a full colostomy. While surgery is not curative for Crohn's disease, 75% of all patients will undergo at least one surgery in their lifetime, and up to 90% of these patients require additional surgeries. A therapeutic agent which can successfully treat inflammatory bowel disease can enormously improve a patient's quality of life, while potentially saving the healthcare system millions of dollars in costs associated with invasive surgical procedures. Identification of such useful therapeutic agents has been hindered because no gene expression profile has been adequately developed that can distinguish between various IBD subtypes.

In view of the above, it is clear that there remains a need in the art for compositions and methods to combat inflammatory bowel diseases, including ulcerative colitis and Crohn's disease.

### SUMMARY OF THE INVENTION

The present invention relates in general to the association of certain biomarkers (e.g., microRNAs) with inflammatory bowel diseases.

In certain embodiments, the invention relates to a method of determining whether a subject is afflicted with an inflammatory bowel disease, condition, or subtype thereof, the method comprising:

a) determining the level of expression or activity of a biomarker listed in Tables 2-14 or a fragment thereof in a subject sample;

b) determining the normal level of expression or activity of the biomarker in a control sample; and

c) comparing the level of expression or activity of said biomarker detected in steps a) and b);

wherein a significant modulation in the level of expression or activity of the biomarker in the subject sample relative to the normal level of expression or activity of the biomarker in a control sample is an indication that the subject is afflicted with an inflammatory bowel disease, condition, or a subtype thereof.

It will be appreciated that the embodiments described herein may be applicable to any of the methods of the invention.

In certain embodiments, the invention relates to any one of the methods described herein, wherein the inflammatory bowel disease, condition, or subtype thereof is selected from the group consisting of active ulcerative colitis, inactive ulcerative colitis, Crohn's disease, irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis, pseudomembranous colitis, ischemic inflammatory bowel disease, Behcet's disease, sarcoidosis, scleroderma, IBD dysplasia, and dysplasia associated masses or lesions.

In certain embodiments, the invention relates to any one of the methods described herein, wherein the sample comprises cells, tissue, blood, plasma, serum, stool, or mucus, obtained from the subject. In certain embodiments, the invention relates to any one of the methods described herein, wherein the subject cells are obtained from the group consisting of stomach tissue, small intestine tissue, colon tissue, and peripheral blood cell subtypes.

In certain embodiments, the invention relates to any one of the methods described herein, wherein the expression level of the biomarker is assessed by detecting the presence in the samples of a polynucleotide molecule encoding the biomarker or a portion of said polynucleotide molecule. In certain embodiments, the invention relates to any one of the methods described herein, wherein the polynucleotide molecule is a mRNA, cDNA, miRNA, or functional variants or fragments thereof. In certain embodiments, the invention relates to any one of the methods described herein, wherein the miRNA or functional variants thereof comprise mature miRNA, pre-miRNA, pri-miRNA, miRNA*, anti-miRNA, or a miRNA binding site. In certain embodiments, the invention relates to any one of the methods described herein, wherein the step of detecting further comprises amplifying the polynucleotide molecule. In certain embodiments, the invention relates to any one of the methods described herein, wherein the expression level of the biomarker is assessed by annealing a nucleic acid probe with the sample of the polynucleotide encoding the biomarker or a portion of said polynucleotide molecule under stringent hybridization conditions.

In certain embodiments, the invention relates to any one of the methods described herein, wherein the expression level of the biomarker is assessed by detecting the presence in the samples of a protein of the biomarker, a polypeptide, or protein fragment thereof comprising said protein. In certain embodiments, the invention relates to any one of the methods described herein, wherein the presence of said protein, polypeptide or protein fragment thereof is detected using a reagent which specifically binds with said protein, polypeptide or protein fragment thereof. In certain embodiments, the invention relates to any one of the methods described herein, wherein the reagent is selected from the group consisting of an antibody, an antibody derivative, and an antibody fragment.

In certain embodiments, the invention relates to any one of the methods described herein, wherein the activity level of the biomarker is assessed by determining the magnitude of modulation of the activity or expression level of downstream targets of the biomarker.

In certain embodiments, the invention relates to any one of the methods described herein, wherein said significant modulation comprises an at least two fold increase or an at least two fold decrease between the expression or activity level of the biomarker in the subject sample relative to the normal expression or activity of the biomarker in the sample from the control subject.

In certain embodiments, the invention relates to a method for monitoring the progression of an inflammatory bowel disease, condition, or a subtype thereof in a subject, the method comprising:

a) detecting in a subject sample at a first point in time the level of expression or activity of a biomarker listed in Tables 2-14 or a fragment thereof;

b) repeating step a) at a subsequent point in time; and

c) comparing the level of expression or activity of said biomarker detected in steps a) and b) to monitor the progression of the inflammatory bowel disease, condition, or subtype thereof.

In certain embodiments, the invention relates to any one of the aforementioned methods, wherein an at least two fold increase or an at least two fold decrease between the expression or activity level of the biomarker in the subject sample at a first point in time relative to the expression or activity level of the biomarker in the subject sample at a subsequent point in time indicates progression of the inflammatory bowel disease.

In certain embodiments, the invention relates to any one of the aforementioned methods, wherein less than a two fold increase or less than a two fold decrease between the expression or activity level of the biomarker in the subject sample at a first point in time relative to the expression or activity level of the biomarker in the subject sample at a subsequent point in time indicates a lack of significant progression of the inflammatory bowel disease.

In certain embodiments, the invention relates to any one of the aforementioned methods, wherein between the first point in time and the subsequent point in time, the subject has undergone treatment to ameliorate the inflammatory bowel disease.

In certain embodiments, the invention relates to a method for predicting the clinical outcome of a patient, the method comprising:

a) assessing the level of expression or activity of a biomarker listed in Tables 2-14 or a fragment thereof in a patient sample;

b) assessing the level of expression or activity of the biomarker in a sample from a control subject having a good clinical outcome; and

c) comparing the level of expression or activity of the biomarker in the patient sample and in the sample from the control subject;

wherein a significantly modulated level of expression or activity in the patient sample as compared to the expression or activity level in the sample from the control subject predicts the clinical outcome of the patient.

In certain embodiments, the invention relates to any one of the aforementioned methods, wherein an at least two fold increase or an at least two fold decrease between the expression or activity level of the biomarker in the subject sample at a first point in time relative to the expression or activity level of the biomarker in the subject sample at a subsequent point in time predicts that the patient has a poor clinical outcome.

In certain embodiments, the invention relates to any one of the aforementioned methods, wherein less than a two fold increase or less than a two fold decrease between the expression or activity level of the biomarker in the subject sample at a first point in time relative to the expression or activity level of the biomarker in the subject sample at a subsequent point in time predicts that the patient has a good clinical outcome.

In certain embodiments, the invention relates to a method of determining the efficacy of a test compound for inhibiting an inflammatory bowel disease, condition, or subtype thereof in a subject, the method comprising comparing:

a) the level of expression or activity of a biomarker listed in Tables 2-14 or a fragment thereof in a first sample obtained from the subject and exposed to the test compound; and

b) the level of expression or activity of the biomarker in a second sample obtained from the subject, wherein the second sample is not exposed to the test compound,

wherein a significantly modulated level of expression or activity of the biomarker, relative to the second sample, is an indication that the test compound is efficacious for inhibiting an inflammatory bowel disease, condition, or subtype thereof in the subject.

In certain embodiments, the invention relates to any one of the aforementioned methods, wherein said significant modulation comprises an at least two fold increase or an at least two fold decrease between the expression or activity level of the biomarker in the first subject sample relative to the second subject sample.

In certain embodiments, the invention relates to any one of the aforementioned methods, wherein the first and second samples are portions of a single sample obtained from the subject or portions of pooled samples obtained from the subject.

In certain embodiments, the invention relates to a method of determining the efficacy of a therapy for inhibiting an inflammatory bowel disease, condition, or subtype thereof in a subject, the method comprising comparing:

a) the level of expression or activity of a biomarker listed in Tables 2-14 or a fragment thereof in a first sample obtained from the subject prior to providing at least a portion of the therapy to the subject, and

b) the level of expression or activity of the biomarker in a second sample obtained from the subject following provision of the portion of the therapy,

wherein a significantly modulated level of expression or activity of the biomarker in the second sample, relative to the first sample, is an indication that the therapy is efficacious for inhibiting the inflammatory bowel disease, condition, or subtype thereof in the subject.

In certain embodiments, the invention relates to any one of the aforementioned methods, wherein said therapy further comprises standard of care therapy for treating the inflammatory bowel disease, condition, or subtype thereof.

In certain embodiments, the invention relates to a method for identifying a compound which inhibits an inflammatory bowel disease, condition, or subtype thereof, the method comprising:

a) contacting a biomarker listed in Tables 2-14 or a fragment thereof with a test compound; and

b) determining the effect of the test compound on the level of expression or activity of the biomarker to thereby identify a compound which inhibits an inflammatory bowel disease, condition, or subtype thereof.

In certain embodiments, the invention relates to any one of the aforementioned methods, wherein an at least two fold increase or an at least two fold decrease between the expression or activity level of the biomarker in the presence of the test compound relative to the expression or activity level of the biomarker in the absence of the test compound identifies a compound which inhibits an inflammatory bowel disease, condition, or subtype thereof.

In certain embodiments, the invention relates to any one of the aforementioned methods, wherein the biomarker is expressed on a cell. In certain embodiments, the invention relates to any one of the aforementioned methods, wherein said cells are isolated from an animal model of an inflammatory bowel disease, condition, or subtype thereof. In certain embodiments, the invention relates to any one of the aforementioned methods, wherein said cells are from a subject afflicted with an inflammatory bowel disease, condition, or subtype thereof.

In certain embodiments, the invention relates to a method for inhibiting an inflammatory bowel disease, condition, or subtype thereof, the method comprising contacting a cell with an agent that modulates the expression or activity level of a biomarker listed in Tables 2-14 or a fragment thereof to thereby inhibit an inflammatory bowel disease, condition, or subtype thereof.

In certain embodiments, the invention relates to any one of the aforementioned methods, wherein the expression or activity of the biomarker is downmodulated.

In certain embodiments, the invention relates to any one of the aforementioned methods, wherein the expression or activity of the biomarker is upmodulated.

In certain embodiments, the invention relates to any one of the aforementioned methods, wherein the step of contacting occurs in vivo, ex vivo, or in vitro.

In certain embodiments, the invention relates to any one of the aforementioned methods, further comprising contacting the immune cell with an additional agent that inhibits an inflammatory bowel disease, condition, or subtype thereof.

In certain embodiments, the invention relates to a method for treating a subject having an inflammatory bowel disease, condition, or subtype thereof, the method comprising administering an agent that modulates the level of expression or activity of a biomarker listed in Tables 2-14 or a fragment thereof such that the inflammatory bowel disease, condition, or subtype thereof is treated.

In certain embodiments, the invention relates to any one of the aforementioned methods, wherein said agent downmodulates the expression or activity of the biomarker.

In certain embodiments, the invention relates to any one of the aforementioned methods, wherein said agent upmodulates the expression or activity of the biomarker.

In certain embodiments, the invention relates to any one of the aforementioned methods, further comprising administering a second agent that treats an inflammatory bowel disease, condition, or subtype thereof.

In certain embodiments, the invention relates to a pharmaceutical composition comprising a polynucleotide encoding a biomarker listed in Tables 2-14 or a fragment thereof in a pharmaceutically acceptable carrier.

In certain embodiments, the invention relates to any one of the aforementioned pharmaceutical compositions, wherein the polynucleotide encoding a biomarker listed in Tables 2-14 or a fragment thereof further comprises an expression vector.

In certain embodiments, the invention relates to a method of using any one of the aforementioned pharmaceutical compositions for treating an inflammatory bowel disease, condition, or subtype thereof.

In certain embodiments, the invention relates to a kit comprising an agent which selectively binds to a biomarker listed in Tables 2-14 or a fragment thereof and instructions for use.

In certain embodiments, the invention relates to a kit comprising an agent which selectively hybridizes to a polynucleotide encoding a biomarker listed in Tables 2-14 or fragment thereof and instructions for use.

In certain embodiments, the invention relates to a kit comprising an agent which mimics a biomarker listed in Tables 2-14 or a fragment thereof and instructions for use.

In certain embodiments, the invention relates to a biochip comprising a solid substrate, said substrate comprising a plurality of probes capable of detecting one or more biomarkers listed in Tables 2-14 or a fragment thereof wherein each probe is attached to the substrate at a spatially defined address.

In certain embodiments, the invention relates to any one of the aforementioned biochips, wherein the probes are complementary to a miRNA listed in Tables 2-14 as differentially expressed in inflammatory bowel diseases, conditions, or subtypes thereof.

### BRIEF DESCRIPTION OF THE DRAWINGS

FIG. 1 shows miRNA expression in human colon tissues. The expression of active UC-associated miRNAs was assessed in healthy control tissues as well as in active UC, inactive UC, IC, IBS, MC, and CD by qRT-PCR. The 6 most highly expressed, active UC-associated miRNAs are shown. Data are presented as box-whisker plots (box, 25%-75%; whisker, 5%-95%; line, median). *P<0.05; **P<0.005; ***P<0.001.

FIG. 2 shows miR-192 and MIP-2α localization in human colon tissues. Dual immunohistochemistry and in situ hybridization were performed on colon biopsy tissues from healthy controls and active UC for MIP-2α and miR-192, respectively. MIP-2α is not detected in epithelial cells of healthy control tissues but is detected in the epithelial cells and lamina propria cells of active UC tissues. miR-192 is localized to colonic epithelial cells of healthy control tissues, but not visible in the epithelial layer of active UC tissues. Green, miR-192; red, MIP-2α; blue, DAPI nuclear staining Pictures were imaged at ×40 magnification at 1024×1024 pixels resolution on a Zeiss LSM 510 Meta™ confocal microscope (20 μm scale). Controls for immunohistochemistry and in situ hybridization are shown in FIG. 8.

FIGS. 3A-3B show MIP-2α expression in human colon tissues and correlation with miR-192 expression. FIG. 3A shows MIP-2α mRNA expression in human colon biopsy tissues by qRT-PCR. Data are presented as MIP-2α expression relative to GAPDH (***P<0.001). FIG. 3B shows correlation of MIP-2α mRNA expression with miR-192 expression in individual biopsy samples from healthy controls (open circles; n=15), active UC (closed circles; n=15), and all other tissues (open squares; n=32).

FIGS. 4A-4C show MIP-2α and associated miRNA expression in TNF-α-stimulated HT29 colonic epithelial cells. FIGS. 4A-4B shows MIP-2α mRNA expression (FIG. 4A) and protein secretion (FIG. 4B) in HT29 cells stimulated with TNF-α at various time points (*P<0.05). FIG. 4C shows the expression of MIP-2α-associated miRNAs was assessed at 1 and 24 hours after TNF-α stimulation. The expression patterns of the 4 most highly expressed miRNAs are demonstrated. *P<0.05; **P<0.005, ***P<0.001.

FIGS. 5A-5C show MIP-2α miRNA binding site mutation effects on reporter expression. FIG. 5A shows a schematic representation of MIP-2α mRNA with putative miRNA binding sites. FIG. 5B shows a sequence alignment and specific miRNA binding site mutations in the pMIR-MIP-2α 3′UTR reporter constructs. FIG. 5C shows luciferase reporter activity in the pMIR-MIP-2α 3′UTR reporter construct and associated miRNA binding site mutations. Luciferase activity (normalized to Renilla luciferase activity) data is presented relative to the pMIR-MIP-2α 3′UTR reporter construct (*P<0.05).

FIGS. 6A-6E show miR-192 inhibition of MIP-2α mRNA and protein expression. FIGS. 6A-6B shows TNF-α-induced MIP-2α mRNA expression (FIG. 6A) and protein secretion (FIG. 6B) were significantly reduced in HT29 cells transfected with an miR-192 mimic. The control mimic had no effect. *P<0.005; **P<0.001. FIG. 6C shows TNF-α-induced RANTES expression was not inhibited by the miR-192 mimic. FIGS. 6D-6E show that TNF-α-induced MIP-2α mRNA expression (FIG. 6D) and protein secretion (FIG. 6E) were also significantly reduced in HT29 cells transfected with a plasmid containing the genomic sequence of pre-miR-192. Transfection of a plasmid containing a scrambled miR-192 sequence had no effect (**P<0.001).

FIG. 7 shows additional miRNA expression in human colon tissues. The expression of active UC-associated miRNAs was assessed in healthy control tissues as well as in active UC, inactive UC, IC, IBS, MC, and CD by qRT-PCR. The remaining 5 active UC-associated miRNAs, not included in FIG. 1, are shown. Data is presented as box-whisker plots (box, 25%-75%; whisker, 5%-95%; line, median). *P<0.05; **P<0.005, ***P<0.001.

FIGS. 8A-8B show MIP-2α immunohistochemistry and miR-192 in situ hybridization controls. FIGS. 8A-8B show the results of dual in situ hybridization (FIG. 8A) and immunohistochemistry (FIG. 8B) controls performed on colon biopsy tissues from active UC. FIG. 8A shows in situ hybridization fluorescence (green) was seen in scattered lamina propria cells in the absence of probe; however, no fluorescence was seen in epithelial cells. FIG. 8B shows that immunohistochemical staining (red) was absent in all cells when using nonspecific goat serum as a control (blue, DAPI nuclear staining) Pictures were imaged at ×40 magnification at 1024×1024 pixels resolution on a Zeiss LSM 510 Meta™ confocal microscope.

FIG. 9 shows MIP-2α-associated miRNA expression in TNF-α-stimulated HT29 colonic epithelial cells. The expression of MIP-2α-associated miRNAs was assessed at 1 and 24 hours after TNF-α stimulation. The expression patterns of the remaining 5 MIP-2α-associated miRNAs, not shown in FIG. 4, are demonstrated. *P<0.05; **P<0.005, ***P<0.001.

FIG. 10 shows representative miRNA microarray results from peripheral blood samples. The differential expression of miRNAs isolated from normal, healthy control patients (columns 1 and 2) compared to patients with active UC (column 3) and active CD (column 4) was assessed. Results indicate that patients with IBD express different peripheral blood miRNAs. These peripheral blood miRNAs are distinct from intestinal tissue-specific miRNAs.

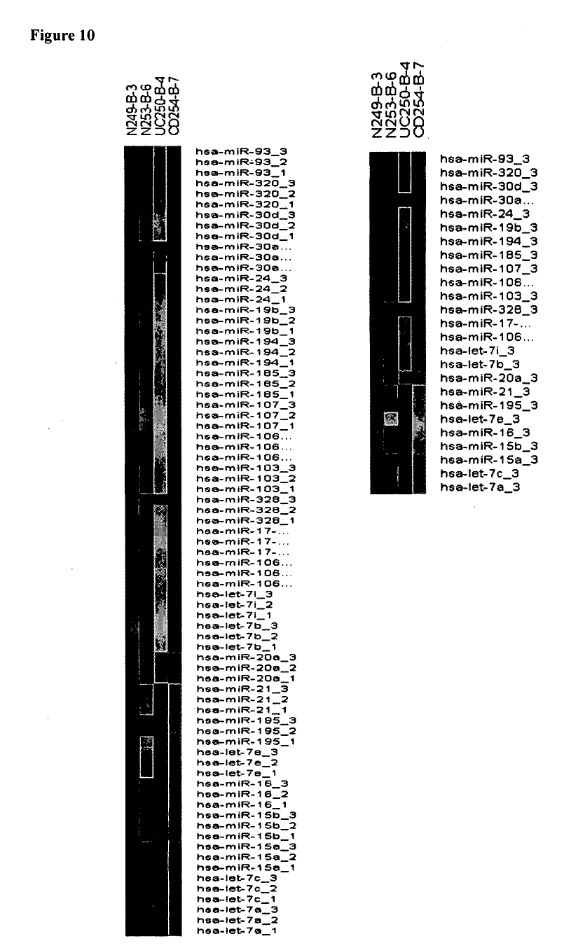

FIG. 11 shows miRNA expression in murine colon tissues. The expression of TNBS-associated miRNAs was assessed in healthy control tissues (Cs) as well as in colon tissues from mice exhibiting TNBS-induced murine colitis. *P<0.005.

FIG. 12 shows the results of in vivo miRNA mimic and inhibitor delivery into colonic tissue of mice.

### BRIEF DESCRIPTION OF THE TABLES

Table 1 shows clinical characteristics of patients who participated in the study.

Table 2 shows miRNAs with binding sites in macrophage inflammatory peptide-2α and their miRNA microarray hybridization intensities in tissues. Hybridization intensities (arbitrary units) are presented as mean values±SE. UC, ulcerative colitis. *P<0.05.

Table 3 shows primers used for qRT-PCR analyses.

Table 4 shows miRNAs differentially expressed in active ulcerative colitis (UC) tissues as compared with normal, healthy controls. Microarray data are presented as mean values±SE in arbitrary units.

Table 5 shows miRNAs differentially expressed in inactive ulcerative colitis (UC) tissues as compared with normal, healthy controls. Microarray data are presented as mean values±SE in arbitrary units.

Table 6 shows miRNAs differentially expressed in active ulcerative colitis (UC) tissues as compared with inactive UC tissues. Microarray data are presented as mean values±SE in arbitrary units.

Table 7 shows relative qRT-PCR levels of active ulcerative colitis (UC)-associated miRNAs. Data are presented as mean values±SE. ^{a}P<0.05. ^{b}P<0.005. ^{c}P<0.001.

Table 8 shows genes differentially expressed in ulcerative colitis (UC) patients versus normal controls.

Table 9 shows miRNAs differentially expressed in Crohn's disease (CD) sigmoid colon biopsy tissues as compared with normal, healthy controls.

Table 10 shows miRNAs differentially expressed in Crohn's disease (CD) terminal ileal biopsy tissues as compared with normal, healthy controls.

Table 11 shows miRNAs differentially expressed in blood samples from subjects having active ulcerative colitis (UC) as compared with normal, healthy controls.

Table 12 shows miRNAs differentially expressed in blood samples from subjects having active Crohn's disease (CD) as compared with normal, healthy controls.

Table 13 shows miRNAs differentially expressed in colon tissues from murine subjects having TNBS-induced colitis as compared with normal, healthy controls.

Table 14 shows the sequences of miRNAs described in Tables 1-13.

### DETAILED DESCRIPTION OF THE INVENTION

The invention is based, at least in part, on the novel discovery that gene and miRNA profiles described herein can be used to distinguish subtypes of inflammatory bowel diseases and related diseases. In addition, the invention provides specific miRNAs that inhibit epithelial cell-derived inflammatory cytokine expression, which are herein identified to be associated with IBD subtypes. Thus, agents such as miRNAs, miRNA analogues, small molecules, RNA interference, aptamer, peptides, peptidomimetics, and antibodies that specifically bind to a biomarker of the invention (e.g., biomarkers listed in Tables 2-14) can be utilized to identify, diagnose, prognose, assess, prevent, and treat inflammatory disease processes, such as IBD, and other related diseases.

### I. Definitions

The articles “a” and “an” are used herein to refer to one or to more than one (i.e. to at least one) of the grammatical object of the article. By way of example, “an element” means one element or more than one element.

Unless otherwise specified here within, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “anti-miRNA” comprises a sequence that is capable of blocking the activity of a miRNA or miRNA* (for example by using LNA-based or morpholino based sequences). The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25 or 26 nucleotides. The sequence of the anti-miRNA may comprise (a) at least 5 nucleotides that are substantially identical to the 5′ of a miRNA and at least 5-12 nucleotide that are substantially complimentary to the flanking regions of the target site from the 5′ end of said miRNA, or (b) at least 5-12 nucleotides that are substantially identical to the 3′ of a miRNA and at least 5 nucleotide that are substantially complimentary to the flanking region of the target site from the 3′ end of said miRNA.

The term “antisense” nucleic acid polypeptide comprises a nucleotide sequence which is complementary to a “sense” nucleic acid encoding a protein, e.g., complementary to the coding strand of a double-stranded cDNA polypeptide, complementary to an mRNA sequence or complementary to the coding strand of a gene. Accordingly, an antisense nucleic acid polypeptide can hydrogen bond to a sense nucleic acid polypeptide.

The term “biochip” refers to a solid substrate comprising an attached probe or plurality of probes of the invention, wherein the probe(s) comprise at least 1, 2, 3, 4, 5, 6, 7, 8, 9, 10, 11, 12, 13, 14, 15, 16, 17, 18, 19, 20, 25, 30, 35, 40, 45, 50, 100, 150, 200 or more probes. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder. The probes may be attached to the biochip in a wide variety of ways, as will be appreciated by those in the art. The probes may either be synthesized first, with subsequent attachment to the biochip, or may be directly synthesized on the biochip. The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing. The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics. The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide. The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit).

The term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “diagnosing IBD” includes the use of the methods, systems, and code of the present invention to determine the presence or absence of IBD in an individual. The term also includes methods, systems, and code for assessing the level of disease activity in an individual. One skilled in the art will know of other methods for evaluating the level of IBD in an individual.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody,” as used herein, is intended to include antibodies made by a non-human cell having variable and constant regions which have been altered to more closely resemble antibodies that would be made by a human cell, for example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

As used herein, the term “inflammatory bowel diseases” or “IBD” includes art-recognized forms of a group of related conditions. Several major forms of IBD are known, and Crohn's disease (regional bowel disease, e.g., inactive and active forms) and ulcerative colitis (e.g., inactive and active forms) are the most common of these disorders. In addition, the IBD encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

As used herein, the term “inhibit” includes the decrease, limitation, or blockage, of, for example a particular action, function, or interaction.

As used herein, the term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules (e.g., miR-192 and MIP-2alpha). Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

An “isolated antibody,” as used herein, is intended to refer to an antibody that is substantially free of other antibodies having different antigenic specificities. Moreover, an isolated antibody may be substantially free of other cellular material and/or chemicals.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g. a package or container) comprising at least one reagent, e.g. a probe, for specifically detecting or modulating the expression of a marker of the invention. The kit may be promoted, distributed, or sold as a unit for performing the methods of the present invention.

A “marker” or “biomarker” includes a nucleic acid or polypeptide whose altered level of expression in a tissue or cell from its expression level in normal or healthy tissue or cell is associated with a disease state, such as a subtype of IBD (e.g., ulcerative colitis). A “marker nucleic acid” is a nucleic acid (e.g., mRNA, cDNA, mature miRNA, pre-miRNA, pri-miRNA, miRNA*, anti-miRNA, or a miRNA binding site, or a variant thereof) and other classes of small RNAs known to a skilled artisan) encoded by or corresponding to a marker of the invention. Such marker nucleic acids include DNA (e.g., cDNA) comprising the entire or a partial sequence of any of the nucleic acid sequences set forth in Tables 2-14 or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” includes a protein encoded by or corresponding to a marker of the invention. A marker protein comprises the entire or a partial sequence of any of the sequences set forth in Tables 2-14. The terms “protein” and “polypeptide” are used interchangeably.

As used herein, the term “modulate” includes up-regulation and down-regulation, e.g., enhancing or inhibiting a response.

As used herein, the term “miRNA” or “miR” means a non-coding RNA of between about 17 and about 25 nucleobases in length which hybridizes to and regulates the expression of a coding RNA. An ˜17-25 nucleotide miRNA molecule can be obtained from a miR precursor through natural processing routes (e.g., using intact cells or cell lysates) or by synthetic processing routes (e.g., using isolated processing enzymes, such as isolated Dicer, Argonaut, or RNAase III). It is understood that the 17-25 nucleotide RNA molecule can also be produced directly by biological or chemical syntheses, without having been processed from a miR precursor. For ease of discussion, the phrase “miR gene expression products” encompasses both miRNAs produced through pre-miRNA processing and miRNAs produced through direct biological or chemical synthesis. A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke at al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

As used herein, the term “miR precursor,” “pre-miRNA,” or “pre-miR” means a non-coding RNA having a hairpin structure, which contains a miRNA. In certain embodiments, a pre-miRNA is the product of cleavage of a primary mi-RNA transcript, or “pri-miR” by the double-stranded RNA-specific ribonuclease known as Drosha, but pre-miRNAs can also be produced directly by biological or chemical synthesis without having been processed from a pri-miR. The pre-miRNA sequence may comprise from 45-90, 60-80 or 60-70 nucleotides. The sequence of the pre-miRNA may comprise a miRNA and a miRNA* as set forth below. The pre-miRNA may also comprise a miRNA or miRNA* and the complement thereof, and variants thereof. The sequence of the pre-miRNA may also be that of a pri-miRNA excluding from 0-160 nucleotides from the 5′ and 3′ ends of the pri-miRNA. The effector miR duplex or single stranded sequence actually loaded into the RISC complex is known as “mature miRNA,” whereas the single stranded sequence of the miR duplex not loaded into the RISC complex is known as “miRNA*”.

As used herein, the term “pri-miRNA” means a primary miRNA transcript that is cleaved by Drosha or an equivalent protein. The pri-miRNA sequence may comprise from 45-250, 55-200, 70-150 or 80-100 nucleotides. The sequence of the pri-miRNA may comprise a pre-miRNA, miRNA and miRNA* as set forth below. The pri-miRNA may also comprise a miRNA or miRNA* and the complement thereof, and variants thereof. The pri-miRNA may form a hairpin structure. The hairpin may comprise a first and second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefte f. Chemie 125: 167-188 (1994), the contents of which are incorporated herein. The hairpin may comprise a terminal loop of 4-20, 8-12 or 10 nucleotides.

The “normal” level of expression of a marker is the level of expression of the marker in cells of a subject, e.g., a human patient, not afflicted with an inflammatory bowel disease. An “over-expression” or “significantly higher level of expression” of a marker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples.

The term “peripheral blood cell subtypes” refers to cell types normally found in the peripheral blood including, but is not limited to, eosinophils, neutrophils, T cells, monocytes, NK cells, granulocytes, and B cells.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of IBD or the likelihood of recovery from the disease. In some embodiments, the use of statistical algorithms provides a prognosis of IBD in an individual. For example, the prognosis can be surgery, development of a clinical subtype of IBD (e.g., ulcerative colitis), development of one or more clinical factors, development of intestinal cancer, or recovery from the disease.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

As used herein, “subject” refers to any healthy animal, mammal or human, or any animal, mammal or human afflicted with an inflammatory bowel disease (e.g., ulcerative colitis) or a related disease. The term “subject” is interchangeable with “patient”.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

A “transcribed polynucleotide” or “nucleotide transcript” is a polynucleotide (e.g. an mRNA, hnRNA, cDNA, mature miRNA, pre-miRNA, pri-miRNA, miRNA*, anti-miRNA, or a miRNA binding site, or a variant thereof or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors.” In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

| GENETIC CODE | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for a fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

### II. Agents that Modulate Immune Cell Activation

The agents of the present invention can modulate, e.g., up- or down-regulate, expression and/or activity of gene products or fragments thereof encoded by biomarkers of the invention, including the biomarkers listed in Tables 2-14, or fragments thereof and, thereby, prevent and treat inflammatory bowel diseases (e.g., ulcerative colitis). Exemplary agents include antibodies, small molecules, peptides, peptidomimetics, natural ligands, and derivatives of natural ligands, that can either activate or inhibit protein biomarkers of the invention, including the biomarkers listed in Tables 2-14, or fragments thereof; RNA interference, antisense, nucleic acid aptamers, that can downregulate the expression and/or activity of the biomarkers of the invention, including the biomarkers listed in Tables 2-14, or fragments thereof; and miRNAs, nucleic acid expression vectors, and that can upregulate the expression and/or activity of the biomarkers of the invention, including the biomarkers listed in Tables 2-14, or fragments thereof.

An isolated polypeptide or a fragment thereof (or a nucleic acid encoding such a polypeptide) corresponding to a biomarker of the invention, including the biomarkers listed in Tables 2-14 or fragments thereof, can be used as an immunogen to generate antibodies that bind to said immunogen, using standard techniques for polyclonal and monoclonal antibody preparation according to well known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

For example, a polypeptide immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well known protocols used for fusing lymphocytes and immortalized cell lines can be applied for the purpose of generating a monoclonal antibody against a biomarker of the invention, including the biomarkers listed in Tables 2-14, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. The use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

Additionally, fully human antibodies could be made against biomarkers of the invention, including the biomarkers listed in Tables 2-14, or fragments thereof. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of a biomarker of the invention, including a biomarker listed in Tables 2-14, or a fragment thereof. In one embodiment, the bispecific antibody could specifically bind to both a polypeptide or a fragment thereof and its natural binding partner(s) or a fragment(s) thereof.

In another aspect of this invention, peptides or peptide mimetics can be used to antagonize or promote the activity of a biomarker of the invention, including a biomarker listed in Tables 2-14, or a fragment(s) thereof. In one embodiment, variants of a biomarker listed in Tables 2-14 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes a biomarker of the invention, including a biomarker listed in Tables 2-14, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivitization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers listed in Tables 2-14 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

The invention also relates to chimeric or fusion proteins of the biomarkers of the invention, including the biomarkers listed in Tables 2-14, or fragments thereof. As used herein, a “chimeric protein” or “fusion protein” comprises a biomarker of the invention, including a biomarker listed in Tables 2-14, or a fragment thereof, operatively linked to another polypeptide having an amino acid sequence corresponding to a protein which is not substantially homologous to the respective biomarker. In a preferred embodiment, the fusion protein comprises at least one biologically active portion of a biomarker of the invention, including a biomarker listed in Tables 2-14, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between a biomarker polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can enhance or upregulate one or more biological activities associated with the corresponding wild-type, naturally occurring, or synthetic small nucleic acids. In another embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, a biomarker of the invention, including a biomarkers listed in Tables 2-14, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin. In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Tables 2-14). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, R. (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl)cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods and compositions presented herein also include RNA endoribonucleases (hereinafter “Cech-type ribozymes”) such as the one which occurs naturally in Tetrahymena thermophile (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g. antibodies, small molecules, peptides, fusion proteins, or small nucleic acids) can be incorporated into pharmaceutical compositions and administered to a subject in vivo. The compositions may contain a single such molecule or agent or any combination of modulatory agents described herein.

### III. Methods of Selecting Agents that Modulate Immune Cell Activation

Another aspect of the invention relates to methods of selecting agents (e.g., antibodies, fusion proteins, peptides, small molecules, or small nucleic acids) which bind to, upregulate, downregulate, or modulate a biomarker of the invention listed in Tables 2-14 and/or an inflammatory bowel disease (e.g., ulcerative colitis). Such methods utilize screening assays, including cell based and non-cell based assays.

In one embodiment, the invention relates to assays for screening candidate or test compounds which bind to or modulate the expression or activity level of, a biomarker of the invention, including a biomarker listed in Tables 2-14, or a fragment thereof. Such compounds include, without limitation, antibodies, proteins, fusion proteins, nucleic acid molecules, and small molecules.

In one embodiment, an assay is a cell-based assay, comprising contacting a cell expressing a biomarker of the invention, including a biomarker listed in Tables 2-14, or a fragment thereof, with a test compound and determining the ability of the test compound to modulate (e.g. stimulate or inhibit) the level of interaction between the biomarker and its natural binding partners as measured by direct binding or by measuring a parameter of inflammatory bowel disease.

For example, in a direct binding assay, the biomarker polypeptide, a binding partner polypeptide of the biomarker, or a fragment(s) thereof, can be coupled with a radioisotope or enzymatic label such that binding of the biomarker polypeptide or a fragment thereof to its natural binding partner(s) or a fragment(s) thereof can be determined by detecting the labeled molecule in a complex. For example, the biomarker polypeptide, a binding partner polypeptide of the biomarker, or a fragment(s) thereof, can be labeled with ¹²⁵I, ³⁵S, ¹⁴C, or ³H, either directly or indirectly, and the radioisotope detected by direct counting of radioemmission or by scintillation counting. Alternatively, the polypeptides of interest a can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to modulate the interactions between a biomarker of the invention, including a biomarker listed in Tables 2-14, or a fragment thereof, and its natural binding partner(s) or a fragment(s) thereof, without the labeling of any of the interactants (e.g., using a microphysiometer as described in McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of the blocking agents (e.g. antibodies, fusion proteins, peptides, nucleic acid molecules, or small molecules) to antagonize the interaction between a given set of polypeptides can be accomplished by determining the activity of one or more members of the set of interacting molecules. For example, the activity of a biomarker of the invention, including a biomarker listed in Tables 2-14, or a fragment thereof, can be determined by detecting induction of cytokine or chemokine response (e.g., downstream effectors of MIP-2alpha), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the biomarker or a fragment thereof. Determining the ability of the blocking agent to bind to or interact with said polypeptide can be accomplished by measuring the ability of an agent to modulate immune responses, for example, by detecting changes in type and amount of cytokine secretion, changes in apoptosis or proliferation, changes in gene expression or activity associated with cellular identity, or by interfering with the ability of said polypeptide to bind to antibodies that recognize a portion thereof.

In yet another embodiment, an assay of the present invention is a cell-free assay in which a biomarker of the invention, including a biomarker listed in Tables 2-14 or a fragment thereof, e.g. a biologically active fragment thereof, is contacted with a test compound, and the ability of the test compound to bind to the polypeptide, or biologically active portion thereof, is determined. Binding of the test compound to the biomarker or a fragment thereof, can be determined either directly or indirectly as described above. Determining the ability of the biomarker or a fragment thereof to bind to its natural binding partner(s) or a fragment(s) thereof can also be accomplished using a technology such as real-time Biomolecular Interaction Analysis (BIA) (Sjolander, S, and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological polypeptides. A biomarker polypeptide or a fragment thereof can be immobilized on a BIAcore chip and multiple agents, e.g., blocking antibodies, fusion proteins, peptides, or small molecules, can be tested for binding to the immobilized biomarker polypeptide or fragment thereof. An example of using the BIA technology is described by Fitz et al. (1997) Oncogene 15:613.

The cell-free assays of the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either the biomarker polypeptide, the natural binding partner(s) polypeptide of the biomarker, or fragments thereof, to facilitate separation of complexed from uncomplexed forms of one or both of the proteins, as well as to accommodate automation of the assay. Binding of a test compound in the assay can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-base fusion proteins, can be adsorbed onto glutathione Sepharose® beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

In an alternative embodiment, determining the ability of the test compound to modulate the activity of a biomarker of the invention, including a biomarker listed in Tables 2-14, or a fragment thereof, or of natural binding partner(s) thereof can be accomplished by determining the ability of the test compound to modulate the expression or activity of a gene, e.g., nucleic acid, or gene product, e.g., polypeptide, that functions downstream of the interaction. For example, inflammation (e.g., cytokine and chemokine) responses can be determined, the activity of the interactor polypeptide on an appropriate target can be determined, or the binding of the interactor to an appropriate target can be determined as previously described.

In another embodiment, modulators of a biomarker of the invention, including a biomarker listed in Tables 2-14, or a fragment thereof, are identified in a method wherein a cell is contacted with a candidate compound and the expression or activity level of the biomarker is determined. The level of expression of biomarker mRNA or polypeptide or fragments thereof in the presence of the candidate compound is compared to the level of expression of biomarker mRNA or polypeptide or fragments thereof in the absence of the candidate compound. The candidate compound can then be identified as a modulator of biomarker expression based on this comparison. For example, when expression of biomarker mRNA or polypeptide or fragments thereof is greater (statistically significantly greater) in the presence of the candidate compound than in its absence, the candidate compound is identified as a stimulator of biomarker expression. Alternatively, when expression of biomarker mRNA or polypeptide or fragments thereof is reduced (statistically significantly less) in the presence of the candidate compound than in its absence, the candidate compound is identified as an inhibitor of biomarker expression. The expression level of biomarker mRNA or polypeptide or fragments thereof in the cells can be determined by methods described herein for detecting biomarker mRNA or polypeptide or fragments thereof.

In yet another aspect of the invention, biomarker of the invention, including a biomarker listed in Tables 2-14, or a fragment thereof, can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other polypeptides which bind to or interact with the biomarker or fragments thereof and are involved in activity of the biomarkers. Such biomarker-binding proteins are also likely to be involved in the propagation of signals by the biomarker polypeptides or biomarker natural binding partner(s) as, for example, downstream elements of a biomarker-mediated signaling pathway.

The two-hybrid system is based on the modular nature of most transcription factors, which consist of separable DNA-binding and activation domains. Briefly, the assay utilizes two different DNA constructs. In one construct, the gene that codes for a biomarker polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming a biomarker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with a biomarker polypeptide of the invention, including a biomarker listed in Tables 2-14 or a fragment thereof.

In another aspect, the invention pertains to a combination of two or more of the assays described herein. For example, a modulating agent can be identified using a cell-based or a cell-free assay, and the ability of the agent to modulate the activity of a biomarker polypeptide or a fragment thereof can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### IV. Pharmaceutical Compositions

Agents that modulate the expression or activity level of a biomarker of the invention, including a biomarker listed in Tables 2-14 or a fragment thereof, including, e.g., blocking antibodies, peptides, fusion proteins, nucleic acid molecules, and small molecules) can be incorporated into pharmaceutical compositions suitable for administration to a subject. Such compositions typically comprise the antibody, peptide, fusion protein or small molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Oral or rectal administration may be particularly effective, because of the greater convenience and acceptability of these routes for treatment of inflammatory bowel diseases. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., blocking antibodies, peptides, fusion proteins, or small molecules that inhibit or enhance the interactions between or activity of a biomarker polypeptide or a fragment thereof and its natural binding partner(s) or a fragment(s) thereof) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by, and directly dependent on, the unique characteristics of the active compound, the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In some embodiments, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity of a biomarker of the invention, including biomarkers listed in Tables 2-14 or fragments thereof. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the scope of knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such polypeptides may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and Thorpe et al. “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The above described modulating agents may be administered it the form of expressible nucleic acids which encode said agents. Such nucleic acids and compositions in which they are contained, are also encompassed by the present invention. For instance, the nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### V. Uses and Methods of the Invention

The biomarkers of the invention, including the biomarkers listed in Tables 2-14 or fragments thereof, described herein, can be used in one or more of the following methods: a) screening assays; b) predictive medicine (e.g., diagnostic assays, prognostic assays, and monitoring clinical trials); and c) methods of treatment (e.g., therapeutic and prophylactic, e.g., by up- or down-modulating the immune response).

The isolated nucleic acid molecules of the invention can be used, for example, to express a biomarker of the invention, including a biomarker listed in Tables 2-14 or a fragment thereof (e.g., via a recombinant expression vector in a host cell in gene therapy applications or synthetic nucleic acid molecule), to detect biomarker mRNA or a fragment thereof (e.g., in a biological sample) or a genetic alteration in a biomarker gene, and to modulate biomarker activity, as described further below. The biomarker polypeptides or fragments thereof can be used to treat conditions or disorders characterized by insufficient or excessive production of a biomarker polypeptide or fragment thereof or production of biomarker polypeptide inhibitors. In addition, the biomarker polypeptides or fragments thereof can be used to screen for naturally occurring biomarker binding partner(s), to screen for drugs or compounds which modulate biomarker activity, as well as to treat conditions or disorders characterized by insufficient or excessive production of biomarker polypeptide or a fragment thereof or production of biomarker polypeptide forms which have decreased, aberrant or unwanted activity compared to biomarker wild-type polypeptides or fragments thereof (e.g., inflammatory bowel diseases such as immune system disorders such as Crohn's disease (regional bowel disease, e.g., inactive and active forms), ulcerative colitis (e.g., inactive and active forms), irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease), Behcet's disease, sarcoidosis, scleroderma, IBD-associated dysplasia, dysplasia associated masses or lesions, and sclerosing cholangitis.

A. Screening Assays

In one aspect, the invention relates to a method for preventing in a subject, a disease or condition associated with an unwanted or less than desirable immune response. Subjects at risk for a disease that would benefit from treatment with the claimed agents or methods can be identified, for example, by any or a combination of diagnostic or prognostic assays known in the art and described herein (see, for example, agents and assays described in III. Methods of Selecting Agents that Modulate Immune Cell Activation).

B. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the expression and/or activity level of biomarkers of the invention, including biomarkers listed in Tables 2-14 or fragments thereof, in the context of a biological sample (e.g., blood, serum, cells, or tissue) to thereby determine whether an individual is afflicted with a disease or disorder, or is at risk of developing a disorder, associated with aberrant or unwanted biomarker expression or activity. The invention also provides for prognostic (or predictive) assays for determining whether an individual is at risk of developing a disorder associated with biomarker polypeptide, nucleic acid expression or activity. For example, mutations in a biomarker gene can be assayed in a biological sample.

Such assays can be used for prognostic or predictive purpose to thereby prophylactically treat an individual prior to the onset of a disorder characterized by or associated with biomarker polypeptide, nucleic acid expression or activity.

Another aspect of the invention pertains to monitoring the influence of agents (e.g., drugs, compounds, and small nucleic acid-based molecules) on the expression or activity of biomarkers of the invention, including biomarkers listed in Tables 2-14, or fragments thereof, in clinical trials. These and other agents are described in further detail in the following sections.

1. Diagnostic Assays

The present invention provides, in part, methods, systems, and code for accurately classifying whether a biological sample is associated with IBD or a clinical subtype thereof. In some embodiments, the present invention is useful for classifying a sample (e.g., from a subject) as an IBD sample using a statistical algorithm and/or empirical data (e.g., the presence or level of an IBD marker).

An exemplary method for detecting the level of expression or activity of a biomarker of the invention, including a biomarker listed in Tables 2-14 or fragments thereof, and thus useful for classifying whether a sample is associated with IBD or a clinical subtype thereof involves obtaining a biological sample from a test subject and contacting the biological sample with a compound or an agent capable of detecting the biomarker (e.g., polypeptide or nucleic acid that encodes the biomarker or fragments thereof) such that the level of expression or activity of the biomarker is detected in the biological sample. In some embodiments, the presence or level of at least two, three, four, five, six, seven, eight, nine, ten, or more biomarkers of the invention are determined in the individual's sample. In certain instances, the statistical algorithm is a single learning statistical classifier system. For example, a single learning statistical classifier system can be used to classify a sample as an IBD (e.g., ulcerative colitis) sample or non-IBD sample based upon a prediction or probability value and the presence or level of at least one IBD marker. The use of a single learning statistical classifier system typically classifies the sample as an IBD (e.g., ulcerative colitis) sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the IBD classification results to a clinician, e.g., a gastroenterologist or a general practitioner.

In another embodiment, the method of the present invention further provides a diagnosis in the form of a probability that the individual has IBD or a clinical subtype thereof. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having IBD or a clinical subtype thereof. In yet another embodiment, the method of the present invention further provides a prognosis of IBD in the individual. For example, the prognosis can be surgery, development of a clinical subtype of IBD (e.g., ulcerative colitis), development of one or more symptoms, development of intestinal cancer, or recovery from the disease. In some instances, the method of classifying a sample as an IBD sample is further based on the symptoms (e.g., clinical factors) of the individual from which the sample is obtained. The symptoms or group of symptoms can be, for example, diarrhea, abdominal pain, cramping, fever, anemia, weight loss, anxiety, depression, and combinations thereof. In some embodiments, the diagnosis of an individual as having IBD or a clinical subtype thereof is followed by administering to the individual a therapeutically effective amount of a drug useful for treating one or more symptoms associated with IBD or the IBD subtype (e.g., ulcerative colitis). Suitable IBD drugs and standard of care treatments include, but are not limited to, aminosalicylates (e.g., mesalazine, sulfasalazine, and the like), corticosteroids (e.g., prednisone), thiopurines (e.g., azathioprine, 6-mercaptopurine, and the like), methotrexate, monoclonal antibodies (e.g., infliximab), free bases thereof, pharmaceutically acceptable salts thereof, derivatives thereof, analogs thereof, and combinations thereof.

In some embodiments, an agent for detecting biomarker mRNA, genomic DNA, or fragments thereof is a labeled nucleic acid probe capable of hybridizing to biomarker mRNA, genomic DNA, or fragments thereof. The nucleic acid probe can be, for example, full-length biomarker nucleic acid, or a portion thereof, such as an oligonucleotide of at least 15, 30, 50, 100, 250 or 500 nucleotides in length and sufficient to specifically hybridize under stringent conditions well known to a skilled artisan to biomarker mRNA or genomic DNA. Other suitable probes for use in the diagnostic assays of the invention are described herein.

A preferred agent for detecting a biomarker listed in Tables 2-14 or a fragment thereof is an antibody capable of binding to the biomarker, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells, and fluids present within a subject. That is, the detection method of the invention can be used to detect biomarker mRNA, polypeptide, genomic DNA, or fragments thereof, in a biological sample in vitro as well as in vivo. For example, in vitro techniques for detection of biomarker mRNA or a fragment thereof include Northern hybridizations and in situ hybridizations. In vitro techniques for detection of biomarker polypeptide include enzyme linked immunosorbent assays (ELISAs), Western blots, immunoprecipitations and immunofluorescence. In vitro techniques for detection of biomarker genomic DNA or a fragment thereof include Southern hybridizations. Furthermore, in vivo techniques for detection of a biomarker polypeptide or a fragment thereof include introducing into a subject a labeled anti-biomarker antibody. For example, the antibody can be labeled with a radioactive marker whose presence and location in a subject can be detected by standard imaging techniques.

In one embodiment, the biological sample contains polypeptide molecules from the test subject. Alternatively, the biological sample can contain mRNA molecules from the test subject or genomic DNA molecules from the test subject. A preferred biological sample is a gastroenterological tissue (e.g., colon or small intestine tissue) sample isolated by conventional means from a subject.

In another embodiment, the methods further involve obtaining a control biological sample from a control subject, contacting the control sample with a compound or agent capable of detecting polypeptide, mRNA, cDNA, mature miRNA, pre-miRNA, pri-miRNA, miRNA*, anti-miRNA, or a miRNA binding site, or a variant thereof, genomic DNA, or fragments thereof of a biomarker listed in Tables 2-14 such that the presence of biomarker polypeptide, mRNA, genomic DNA, or fragments thereof, is detected in the biological sample, and comparing the presence of biomarker polypeptide, mRNA, cDNA, mature miRNA, pre-miRNA, pri-miRNA, miRNA*, anti-miRNA, or a miRNA binding site, or a variant thereof, genomic DNA, or fragments thereof in the control sample with the presence of biomarker polypeptide, mRNA, cDNA, mature miRNA, pre-miRNA, pri-miRNA, miRNA*, anti-miRNA, or a miRNA binding site, or a variant thereof, genomic DNA, or fragments thereof in the test sample.

The invention also encompasses kits for detecting the presence of a polypeptide, mRNA, cDNA, mature miRNA, pre-miRNA, pri-miRNA, miRNA*, anti-miRNA, or a miRNA binding site, or a variant thereof, genomic DNA, or fragments thereof, of a biomarker listed in Tables 2-14 in a biological sample. For example, the kit can comprise a labeled compound or agent capable of detecting a biomarker polypeptide, mRNA, cDNA, mature miRNA, pre-miRNA, pri-miRNA, miRNA*, anti-miRNA, or a miRNA binding site, or a variant thereof, genomic DNA, or fragments thereof, in a biological sample; means for determining the amount of the biomarker polypeptide, mRNA, cDNA, mature miRNA, pre-miRNA, pri-miRNA, miRNA*, anti-miRNA, or a miRNA binding site, or a variant thereof, genomic DNA, or fragments thereof, in the sample; and means for comparing the amount of the biomarker polypeptide, mRNA, cDNA, mature miRNA, pre-miRNA, pri-miRNA, miRNA*, anti-miRNA, or a miRNA binding site, or a variant thereof, genomic DNA, or fragments thereof, in the sample with a standard. The compound or agent can be packaged in a suitable container. The kit can further comprise instructions for using the kit to detect the biomarker polypeptide, mRNA, cDNA, mature miRNA, pre-miRNA, pri-miRNA, miRNA*, anti-miRNA, or a miRNA binding site, or a variant thereof, genomic DNA, or fragments thereof.

2. Prognostic Assays

The diagnostic methods described herein can furthermore be utilized to identify subjects having or at risk of developing a disease or disorder associated with aberrant expression or activity of a biomarker of the invention, including a biomarker listed in Tables 2-14, or a fragment thereof. As used herein, the term “aberrant” includes biomarker expression or activity levels which deviates from the normal expression or activity in a control.

The assays described herein, such as the preceding diagnostic assays or the following assays, can be utilized to identify a subject having or at risk of developing a disorder associated with a misregulation of biomarker activity or expression, such as in an inflammatory bowel disease (e.g., ulcerative colitis). Alternatively, the prognostic assays can be utilized to identify a subject having or at risk for developing a disorder associated with a misregulation of biomarker activity or expression, such as in an inflammatory bowel disease (e.g., ulcerative colitis). Thus, the present invention provides a method for identifying and/or classifying a disease associated with aberrant expression or activity of a biomarker of the invention, including a biomarker listed in Tables 2-14, or a fragment thereof. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with aberrant biomarker expression or activity. For example, such methods can be used to determine whether a subject can be effectively treated with an agent for an inflammatory bowel disease (e.g., ulcerative colitis). Thus, the present invention provides methods for determining whether a subject can be effectively treated with an agent for a disease associated with aberrant biomarker expression or activity in which a test sample is obtained and biomarker polypeptide or nucleic acid expression or activity is detected (e.g., wherein a significant increase or decrease in biomarker polypeptide or nucleic acid expression or activity relative to a control is diagnostic for a subject that can be administered the agent to treat a disorder associated with aberrant biomarker expression or activity). In some embodiments, significant increase or decrease in biomarker expression or activity comprises at least 2 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher or lower, respectively, than the expression activity or level of the marker in a control sample.

The methods of the invention can also be used to detect genetic alterations in a biomarker of the invention, including a biomarker listed in Tables 2-14 or a fragment thereof, thereby determining if a subject with the altered biomarker is at risk for a disease (e.g., inflammatory bowel disease) characterized by aberrant biomarker activity or expression levels. In preferred embodiments, the methods include detecting, in a sample of cells from the subject, the presence or absence of a genetic alteration characterized by at least one alteration affecting the integrity of a gene encoding a biomarker polypeptide, or the mis-expression of the biomarker. For example, such genetic alterations can be detected by ascertaining the existence of at least one of 1) a deletion of one or more nucleotides from a biomarker gene, 2) an addition of one or more nucleotides to a biomarker gene, 3) a substitution of one or more nucleotides of a biomarker gene, 4) a chromosomal rearrangement of a biomarker gene, 5) an alteration in the level of a messenger RNA transcript of a biomarker gene, 6) aberrant modification of a biomarker gene, such as of the methylation pattern of the genomic DNA, 7) the presence of a non-wild type splicing pattern of a messenger RNA transcript of a biomarker gene, 8) a non-wild type level of a biomarker polypeptide, 9) allelic loss of a biomarker gene, and 10) inappropriate post-translational modification of a biomarker polypeptide. As described herein, there are a large number of assays known in the art which can be used for detecting alterations in a biomarker gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker gene (see Abravaya et al. (1995) Nucleic Acids Res. 23:675-682). This method can include the steps of collecting a sample of cells from a subject, isolating nucleic acid (e.g., genomic, mRNA, mature miRNA, pre-miRNA, pri-miRNA, miRNA*, anti-miRNA, or a miRNA binding site, or a variant thereof) from the cells of the sample, contacting the nucleic acid sample with one or more primers which specifically hybridize to a biomarker gene of the invention, including the biomarker genes listed in Tables 2-14, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a biomarker gene of the invention, including a biomarker listed in Tables 2-14, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a biomarker gene of the invention, including a gene listed in Tables 2-14, or a fragment thereof, can be identified by hybridizing a sample and control nucleic acids, e.g., DNA, RNA, mRNA, mature miRNA, pre-miRNA, pri-miRNA, miRNA*, anti-miRNA, or a miRNA binding site, or a variant thereof, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in a biomarker can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a biomarker gene of the invention, including a gene listed in Tables 2-14, or a fragment thereof, and detect mutations by comparing the sequence of the sample biomarker gene with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad. Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene of the invention, including a gene listed in Tables 2-14, or fragments thereof, include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker genes of the invention, including genes listed in Tables 2-14, or fragments thereof, obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in biomarker genes of the invention, including genes listed in Tables 2-14, or fragments thereof. For example, single strand conformation polymorphism (SSCP) may be used to detect differences in electrophoretic mobility between mutant and wild type nucleic acids (Orita et al. (1989) Proc Natl. Acad. Sci. USA 86:2766; see also Cotton (1993) Mutat. Res. 285:125-144 and Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a biomarker of the invention, including a biomarker listed in Tables 2-14, or fragments thereof.

3. Monitoring of Effects During Clinical Trials

Monitoring the influence of agents (e.g., drugs) on the expression or activity of a biomarker of the invention, including a biomarker listed in Tables 2-14, or a fragment thereof (e.g., the modulation of inflammatory bowel disease state) can be applied not only in basic drug screening, but also in clinical trials. For example, the effectiveness of an agent determined by a screening assay as described herein to increase expression and/or activity of a biomarker of the invention, including a biomarker listed in Tables 2-14 or a fragment thereof, can be monitored in clinical trials of subjects exhibiting decreased expression and/or activity of a biomarker of the invention, including a biomarker of the invention, including a biomarker listed in Tables 2-14, or a fragment thereof, relative to a control reference. Alternatively, the effectiveness of an agent determined by a screening assay to decrease expression and/or activity of a biomarker of the invention, including a biomarker listed in Tables 2-14, or a fragment thereof, can be monitored in clinical trials of subjects exhibiting decreased expression and/or activity of the biomarker of the invention, including a biomarker listed in Tables 2-14 or a fragment thereof relative to a control reference. In such clinical trials, the expression and/or activity of the biomarker can be used as a “read out” or marker of the phenotype of a particular cell.

In some embodiments, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting the level of expression and/or activity of a biomarker of the invention, including a biomarker listed in Tables 2-14 or fragments thereof in the preadministration sample; (iii) obtaining one or more post-administration samples from the subject; (iv) detecting the level of expression or activity of the biomarker in the post-administration samples; (v) comparing the level of expression or activity of the biomarker or fragments thereof in the pre-administration sample with the that of the biomarker in the post administration sample or samples; and (vi) altering the administration of the agent to the subject accordingly. For example, increased administration of the agent may be desirable to increase the expression or activity of a biomarker to higher levels than detected (e.g., to increase the effectiveness of the agent.) Alternatively, decreased administration of the agent may be desirable to decrease expression or activity of the biomarker to lower levels than detected (e.g., to decrease the effectiveness of the agent.) According to such an embodiment, biomarker expression or activity may be used as an indicator of the effectiveness of an agent, even in the absence of an observable phenotypic response.

D. Methods of Treatment

The present invention provides for both prophylactic and therapeutic methods of treating a subject at risk of (or susceptible to) a disorder characterized by insufficient or excessive production of biomarkers of the invention, including biomarkers listed in Tables 2-14 or fragments thereof, which have aberrant expression or activity compared to a control. Moreover, agents of the invention described herein can be used to detect and isolate the biomarkers or fragments thereof, regulate the bioavailability of the biomarkers or fragments thereof, and modulate biomarker expression levels or activity.

1. Prophylactic Methods

In one aspect, the invention provides a method for preventing in a subject, a disease or condition associated with an aberrant expression or activity of a biomarker of the invention, including a biomarker listed in Tables 2-14 or a fragment thereof, by administering to the subject an agent which modulates biomarker expression or at least one activity of the biomarker. Subjects at risk for a disease or disorder which is caused or contributed to by aberrant biomarker expression or activity can be identified by, for example, any or a combination of diagnostic or prognostic assays as described herein. Administration of a prophylactic agent can occur prior to the manifestation of symptoms characteristic of the biomarker expression or activity aberrancy, such that a disease or disorder is prevented or, alternatively, delayed in its progression.

2. Therapeutic Methods

Another aspect of the invention pertains to methods of modulating the expression or activity or interaction with natural binding partner(s) of a biomarker of the invention, including a biomarker listed in Tables 2-14 or fragments thereof, for therapeutic purposes. The biomarkers of the invention have been demonstrated to correlate with inflammatory bowel disease (e.g., ulcerative colitis). Accordingly, the activity and/or expression of the biomarker, as well as the interaction between a biomarker or a fragment thereof and its natural binding partner(s) or a fragment(s) thereof can be modulated in order to modulate the immune response.

Modulatory methods of the invention involve contacting a cell with a biomarker of the invention, including a biomarker of the invention, including a biomarker listed in Tables 2-14 or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, a biomarker agonist or antagonist, a peptidomimetic of a biomarker agonist or antagonist, a biomarker peptidomimetic, other small molecule, or small RNA directed against or a mimic of a biomarker nucleic acid gene expression product.

An agent that modulates the expression of a biomarker of the invention, including a biomarker of the invention, including a biomarker listed in Tables 2-14 or a fragment thereof is, e.g., an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule, triplex oligonucleotide, ribozyme, or recombinant vector for expression of a biomarker polypeptide. For example, an oligonucleotide complementary to the area around a biomarker polypeptide translation initiation site can be synthesized. One or more antisense oligonucleotides can be added to cell media, typically at 200 μg/ml, or administered to a patient to prevent the synthesis of a biomarker polypeptide. The antisense oligonucleotide is taken up by cells and hybridizes to a biomarker mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, preferably, such modulation occurs by a means other than by knocking out the biomarker gene.

Agents which modulate expression, by virtue of the fact that they control the amount of biomarker in a cell, also modulate the total amount of biomarker activity in a cell.

In one embodiment, the agent stimulates one or more activities of a biomarker of the invention, including a biomarker listed in Tables 2-14 or a fragment thereof. Examples of such stimulatory agents include active biomarker polypeptide or a fragment thereof and a nucleic acid molecule encoding the biomarker or a fragment thereof that has been introduced into the cell (e.g., mature miRNA, pre-miRNA, pri-miRNA, miRNA*, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, anti-biomarker antibodies, biomarker inhibitors, and compounds identified in the screening assays described herein.

These modulatory methods can be performed in vitro (e.g., by contacting the cell with the agent) or, alternatively, by contacting an agent with cells in vivo (e.g., by administering the agent to a subject). As such, the present invention provides methods of treating an individual afflicted with a condition or disorder that would benefit from up- or down-modulation of a biomarker of the invention listed in Tables 2-14 or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering a biomarker polypeptide or nucleic acid molecule as therapy to compensate for reduced, aberrant, or unwanted biomarker expression or activity.

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In addition, these modulatory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for inflammatory bowel diseases well known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regiment and dosages of these aforementioned chemotherapeutic drugs that are therapeutically effective will depend on the particular immune disorder, e.g., inflammatory bowel disease, being treated, the extent of the disease and other factors familiar to the physician of skill in the art and can be determined by the physician.

### V. Administration of Agents

The immune modulating agents of the invention are administered to subjects in a biologically compatible form suitable for pharmaceutical administration in vivo, to either enhance or suppress immune cell mediated immune responses. By “biologically compatible form suitable for administration in vivo” is meant a form of the protein to be administered in which any toxic effects are outweighed by the therapeutic effects of the protein. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of a blocking antibody may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agents of the invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the invention (e.g., an antibody, peptide, fusion protein or small molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, an agent of the invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays. In addition, an antibody of the invention can also be administered in combination therapy with, e.g., chemotherapeutic agents, hormones, antiangiogens, radiolabelled, compounds, or with surgery, cryotherapy, and/or radiotherapy. An antibody of the invention can also be administered in conjunction with other forms of conventional therapy, either consecutively with, pre- or post-conventional therapy. For example, the antibody can be administered with a therapeutically effective dose of chemotherapeutic agent. In another embodiment, the antibody can be administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regiment and dosages of these aforementioned chemotherapeutic drugs that are therapeutically effective will depend on the particular immune disorder, e.g., Hodgkin lymphoma, being treated, the extent of the disease and other factors familiar to the physician of skill in the art and can be determined by the physician.

In addition, the agents of the invention described herein can be administered using nanoparticle-based composition and delivery methods well known to the skilled artisan. For example, nanoparticle-based delivery for improved nucleic acid (e.g., small RNAs) therapeutics are well known in the art (Expert Opinion on Biological Therapy 7:1811-1822).

This invention is further illustrated by the following examples which should not be construed as limiting. The contents of all references, patents and published patent applications cited throughout this application, as well as the Figures, are incorporated herein by reference.

### EXAMPLES

### Example 1

### Materials and Methods Used in Examples 2-6

### A. Human Tissues

Colonoscopic pinch biopsies from the sigmoid colon of patients with chronic active ulcerative colitis (UC), chronic inactive UC, chronic active colonic Crohn's disease (CD), irritable bowel syndrome (IBS), infectious colitis (IC), microscopic colitis (MC), and normal, healthy patients undergoing screening colonoscopies (Table 1) were obtained using a protocol approved by The Johns Hopkins University Institutional Review Board. In total, 62 patient biopsies were assessed. The diagnoses of active UC, inactive UC, MC (2 cases of collagenous colitis and 1 case of lymphocytic colitis), and CD were confirmed by histopathology conducted on biopsies taken within 10 cm. IC (3 cases of Clostridium difficile colitis and 1 case of Salmonella) were confirmed by microbiological analysis of stool.

B. Total RNA and miRNA Enrichment

Biopsies were placed immediately into 1 mL of TRIzol reagent (Invitrogen, Carlsbad, Calif.) and total RNA was extracted. Small RNA molecules were separated from large RNA fragments (>200 nucleotides) using the PureLink miRNA Isolation Kit™ (Invitrogen) and stored at −80° C. The RediPlate 96 RiboGreen RNA Quantitation Kit™ (Invitrogen) was used to quantitate the RNA molecules in each sample.

C. miRNA Microarray

The NCode Multi-Species miRNA Microarray V2™ (Invitrogen) slides, containing 3 replicate subarrays each, were used to assess miRNA expression in individual small RNA samples from each patient. A total of 58 arrays were assessed. Four biopsy samples (2 IBS, 1 MC, and 1 IC) were assessed only for quantitative reverse transcriptase polymerase chain reaction (qRT-PCR) owing to insufficient small RNA to conduct both miRNA microarray and subsequent validation RTPCR. Briefly, 500 ng of small miRNAs, mixed with NCode miRNA Microarray Controls™, were labeled with the Flashtag RNA labeling Kit™ (Genisphere, Hatfield, Pa.). The Oyster-550-tagged small RNAs were hybridized to the NCode miRNA microarray slides at 52° C. for 16 hours. The arrays were scanned with a GenePix 4000B scanner (Molecular Devices, Downingtown, Pa.) and raw hybridization intensities obtained. The background subtracted median fluorescence intensity was used for normalization using dChip software (available on the world wide web at dchip.org (Li and Wong (2001) Proc. Natl. Acad. Sci. U.S.A. 98, 31-36)). When comparing 2 groups, findings were considered significant if (1) fold change >1.5, (2) t test, P<0.05, (3) the difference between 2 groups means >100 arbitrary units, and (4) mean fluorescence intensity in either group >200 arbitrary units.

D. qRT-PCR for miRNA and mRNA

The NCode SYBR Green miRNA qRT-PCR Kit™ (Invitrogen) and the SYBR Green PCR Master Mix™ (Applied Biosystems, Foster City, Calif.) were used to confirm the miRNA and mRNA expression changes, respectively. For quantitative real-time PCR (qRT-PCR) on biopsy tissues, 200 ng of small RNA was converted to cDNA. The expression of each target miRNA in tissues was calculated relative to Let-7a and Let-7b, 2 highly and ubiquitously expressed miRNAs previously used as control miRNAs (Ro et al. (2006) Biochem. Biophys. Res. Commun. 351, 756-763). For qRT-PCR on the HT29 cell line, total RNA was converted to cDNA. The expression of each target miRNA in the HT29 cell line was calculated relative to U6, a ubiquitously expressed small nuclear RNA. For miRNA qPCR, the reverse primer was the NCode miRNA universal qPCR Primer™ (Invitrogen). Forward miRNA primers and mRNA primers were obtained (Operon; Table 3). A comparative threshold cycle method was used to compare each condition with controls (User Bulletin #2: ABI PRISM 7700 sequence detection system, Perkin-Elmer Corporation, Boston (1997) 11-15).

E. Dual Immunohistochemistry and miRNA in Situ Hybridization

In situ hybridization for miR-192 was performed on cryosections of sigmoid biopsies using a 5′-end digoxigenin-labeled Locked Nucleic Acid-modified mirCURY™ miR-192 detection probe, scrambled control probe or no probe (Exiqon, Vedbaek, Denmark) following manufacturer's recommendations (Kloosterman et al. (2006) Nat. Methods 3, 27-29). Briefly, 10- to 12-μm cryosections were fixed in 4% paraformaldehyde in phosphate-buffered saline (PBS) at 4° C. for 10 minutes then hybridized with 20 nmol/L of the selected detection probes in hybridization buffer (50% formamide, 0.3 mol/L NaCl, 5 mmol/L EDTA, 10% dextran sulfate, 1×Denhardt's, 0.5 mg/mL yeast RNA, 10 mmol/L Na₂PO₄/NaHPO₄, 20 mmol/L Tris-HCl, pH 8.0) overnight at 52° C. After washing and blocking with 0.5% blocking powder (Roche, Nutley, N.J.), 10% sheep serum, and 0.1% Tween-20 in PBS, the sections were incubated with an anti-digoxigenin-fluorescein Fab fragment (1:200, Roche) and goat anti-human MIP-2α immunoglobulin (Ig)G (2 μg/mL, Santa Cruz Biotechnology, Santa Cruz, Calif.) for 1 hour at room temperature. Normal goat serum (Santa Cruz Biotechnology) was used as the negative control. After washing in PBS, the sections were incubated with rabbit anti-FITC-Alexa488 (1:250; Invitrogen) and donkey anti-goat-IgG-Texas Red (1 μg/mL; Santa Cruz Biotechnology). After washing, the sections were counterstained with 4′,6-diamidino-2-phenylindole (DAPI; 1 μg/mL; Molecular Probes, Eugene, Oreg.) for 1 minute. Images were captured with a Zeiss LSM510METAT™ confocal microscope (Zeiss, Oberkochen, Germany).

### F. Human Genome-Wide Microarray

The Human Genome U133 Plus 2.0 Array™ (Affymetrix, Santa Clara, Calif.) was used to compare expression differences in pooled large RNA samples from UC (6 patients) and normal healthy controls (5 patients) as described previously (Lawrance et al. (2001) Hum. Mol. Gen. 10, 445-456). The raw output data of each array was normalized using the dChip software (available on the world wide web at dchip.org) for array-to-array comparison. Fluorescence intensities that demonstrated a >2-fold difference were considered significant. The complete dataset is available at the NCBI Gene Expression Omnibus available on the world wide web at ncbi.nlm.nih.gov/geo, accession number 10791.

### G. Tissue Culture

HT29 cells were cultured in Dulbecco's modified Eagle medium (DMEM) supplemented with 10% fetal bovine serum, penicillin, and streptomycin in a 5% CO₂ incubator. Inflammatory cytokine-induced miRNA experiments were conducted on postconfluent cells that were incubated overnight in DMEM (without serum or antibiotics) then treated with TNF-α (R&D Systems, Minneapolis, Minn.) at 10 ng/mL.

### H. Enzyme-Linked Immunosorbent Assay

The enzyme-linked immunosorbent assay (ELISA) procedures followed a standard protocol. Briefly, 96-well plates were precoated with a rabbit anti-human MIP-2α polyclonal antibody (MBL; 100 ng/well). After washing and blocking (1% sucrose, 1% bovine serum albumin, and 0.9% NaCl), 100 μL of cell culture medium was incubated for 2 hours at room temperature. Rabbit anti-human MIP-2α polyclonal antibody conjugated to biotin (20 ng/well; Antigenix America, Huntington Station, N.Y.) was used as the detection antibody. After incubation with streptavidin-horseradish peroxidase (Invitrogen) and tetramethylbenzidine (Zymed Laboratories, San Francisco, Calif.), 1 N HCl was added to stop the color development. The optical density of the color was measured by a microplate reader at 450 nm. Results were calculated by averaging the duplicate reading for each sample and subtracting the optical density of a blank well.

### I. MIP-2α 3′UTR Construct and Luciferase Report Assay

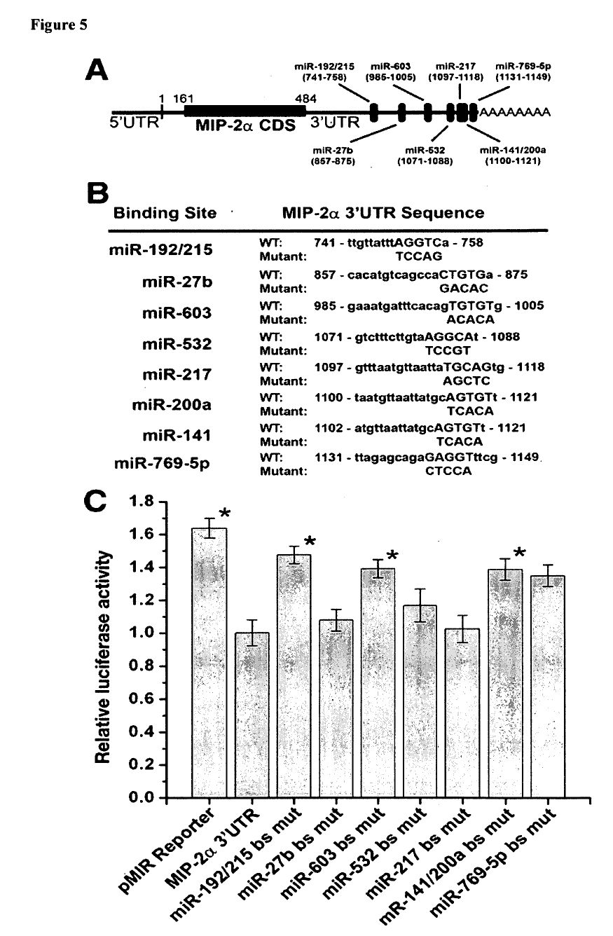

The 3′UTR of MIP-2α mRNA bearing miRNA-binding sites (corresponding to 699-1154 nucleotides of RefSeq NM_{—}002089.3) was cloned into the PmeI and Sad sites downstream of the firefly luciferase reporter vector, pMIR-Report (Ambion, Inc, Austin, Tex.), according to manufacturer's instructions. The QuikChange II Site-Directed Mutagenesis Kit™ (Stratagene, La Jolla, Calif.) was used to create mutant miRNA binding sites in the pMIR-3′UTR vector. Overall, 7 mutants were generated corresponding to the predicted binding sites on MIP-2α 3′UTR for miR-192/215, miR-27b, miR-603, miR-532, miR-217, miR-141/200a, and miR-769-5p. For each miRNA binding site, 5 nucleotides in the 5′ seeding region were substituted as detailed in FIG. 5B.

HT29 cells were cultured in 24-well plates. Each pMIR construct (400 ng/well), along with the Renilla luciferase control plasmid, phRL-CMV (Promega, Madison, Wis.; 1.5 ng/well), was transfected into cells using Lipofectamine 2000 (Invitrogen) according to the manufacturer's guidelines. Cells were harvested 48 hours posttransfection and luciferase levels measured using the Dual Luciferase Reporter Assay System™ (Promega) according to the manufacturer's instructions. Experiments were performed in quadruplicate.

J. miRNA Mimic and Expression Construct Transfection

The miRIDIAN miRNA™ mimics to miR-192 and the cel-miR-67 negative control were obtained from Dharmacon Inc (Lafayette, Colo.). HT29 cells in DMEM containing 10% fetal bovine serum were placed into 12-well plates at 90% confluence at 37° C. in a 5% CO₂ incubator. After 6 hours, the culture media was replaced with DMEM (without serum and antibiotics) containing varying amounts of miRNA mimics and the transfection agent, DharmaFECT-4™ (Dharmacon), according to manufacturer's instructions. The transfected cells were incubated at 37° C. in a 5% CO₂ incubator overnight. The cells were then treated with TNF-α for 4 and 24 hours. The culture media was collected for MIP-2α secretion by ELISA and the total RNA was isolated using TRIzol to detect gene expression changes. The genomic sequence of pre-miR-192 was inserted into the BamH I and Xho I sites of pRNAT-CMV3.2/Hygro expression vector (GenScript, Piscataway, N.J.). An additional control plasmid was constructed by substituting the mature miR-192 sequence with scramble nucleotides and cloning it into the same parental vector. HT29 cells were cultured in 12-well plates overnight. Plasmid DNA (1.5 μg/well) was transfected into cells using Lipofectamine 2000 and incubated at 37° C. in a 5% CO₂ incubator for 48 hours. The cells were then treated with TNF-α for 4 and 24 hours. The culture media was collected for MIP-2α secretion by ELISA and the total RNA was isolated using TRIzol to detect gene expression changes.

### K. Statistical Analysis

Experimental results are expressed as mean values±standard error. Statistical analyses for ELISA and qRT-PCR were performed with the unpaired, 2-tailed Student t tests and 1-way ANOVA for comparing all pairs of groups (SPSS software, version 2.0). P<0.05 was considered significant.

### Example 2

### miRNAs are Differentially Expressed in UC Tissue

Chronic inflammatory bowel diseases such as ulcerative colitis (UC) are associated with differential expression of genes involved in inflammation and tissue remodeling. MicroRNAs, which direct mRNA degradation and translational inhibition, influence a number of disease processes. Thus, it was first sought to determine whether miRNAs are differentially expressed in active UC. Sigmoid colon pinch biopsies from patients with histologically confirmed active UC, inactive UC, and normal, healthy control subjects were obtained. Additional control groups used for comparison included patients with IBS, IC, MC, and CD. Clinical characteristics of each patient group are listed in Table 1.

An miRNA microarray capable of measuring the expression of 553 known human miRNA genes was used to compare miRNA expression among collected samples. A total of 58 miRNA microarrays were performed and analyzed using relatively low-stringency criteria to maximize the identification of candidate miRNAs. A comparison of active UC tissues with healthy control tissues identified an initial 18 miRNAs with a differential expression pattern (Table 4). A comparison of inactive UC tissues with healthy control tissues identified 12 differentially expressed miRNAs (Table 5). Finally, a comparison of inactive UC tissues to active UC tissues identified 6 differentially expressed miRNAs (Table 6).

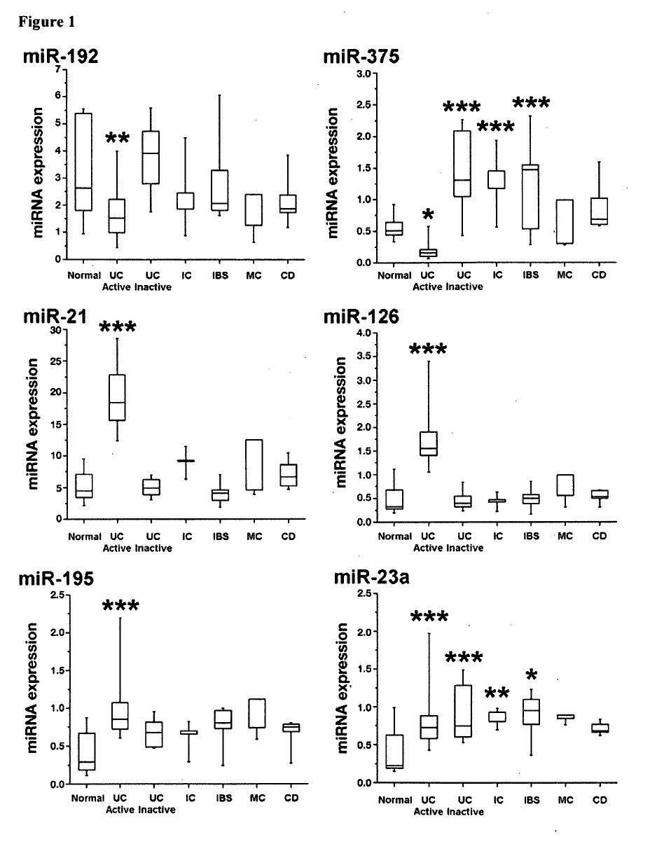

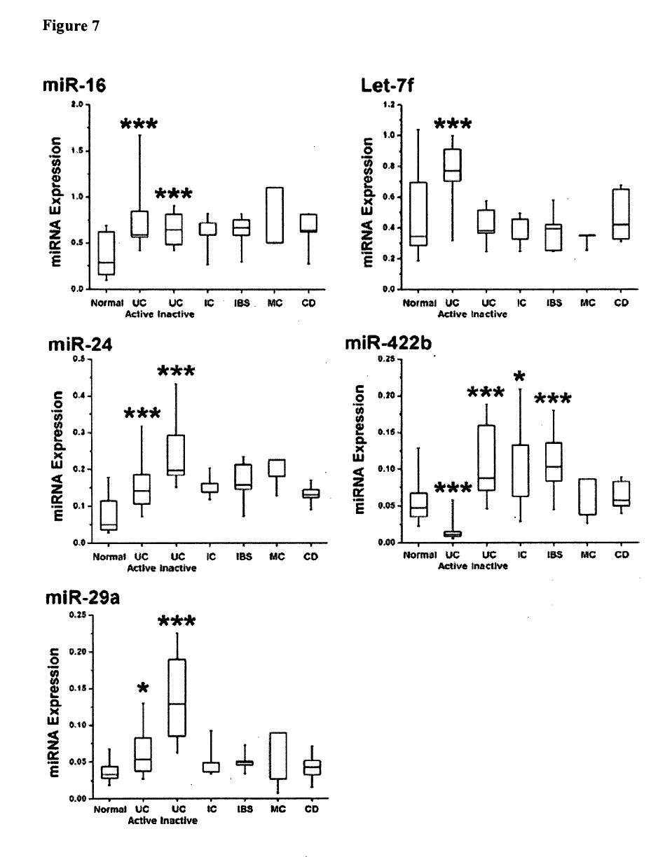

To confirm the differential expression of the candidate miRNAs, subsequent qRT-PCR validation was performed. The miRNAs differentially expressed in active UC tissues as compared with healthy control tissues were focused upon. All 18 active UC-associated miRNAs were subjected to the validation screen. Three miRNAs, miR-192, miR-375, and miR-422b (also referred to as miR-378), were confirmed to be significantly decreased in active UC tissues, whereas 8 miRNAs (miR-16, miR-21, miR-23a, miR-24, miR-29a, miR-126, miR-195, and Let-7f) were significantly increased in active UC tissues, as compared with healthy control tissues. The differential expression of the 6 most highly expressed miRNAs is depicted in FIG. 1. Additional data is included in Table 7 and FIG. 7. Although initially identified in the miRNA microarray as differentially expressed in active UC tissues, qRT-PCR did not confirm the differential expression of the other 7 miRNAs.

Both the miRNA microarray analysis and qRT-PCR identified miR-192 and miR-21 as the most highly expressed of the active UC-associated miRNAs in human colon tissues (FIG. 1). The expression of miR-192 decreased by 47.1% in active UC tissues as compared to healthy control tissues (P<0.005). In contrast, the expression of miR-21 increased by 354.6% in active UC tissues as compared with healthy control tissues (P<0.001).

Further miRNA microarray analysis and validation qRT-PCR conducted on the additional comparison groups (inactive UC, IBS, IC, MC, and CD) revealed distinct differences in expression patterns of the active UC-associated miRNAs (FIG. 1). In inactive UC tissues, all 3 miRNAs with decreased expression in active UC demonstrated different expression patterns. Specifically, in inactive UC, miR-192 was unchanged whereas miR-375 and miR-422b (also known as miR-378) were increased as compared with healthy control tissues (P<0.001). Similarly, although the increased expression of miR-23a, miR-16, miR-24, and miR-29a in inactive UC tissues was similar to that seen in active UC tissues, the expression of miR-21, miR-126, miR-195, and Let-7f was more consistent with the levels seen in healthy control tissues. These results, combined with the observation that 6 other miRNAs are differentially expressed in active UC as compared with inactive UC, indicate that the expression of miRNAs in active and inactive UC are distinct.

In both IC and IBS tissues, miR-375, miR-422b (also known as miR-378), and miR-23a were differentially expressed as compared with healthy control tissues. The pattern of expression of these miRNAs was more similar to inactive UC tissues than active UC, with all 3 demonstrating increased expression. In MC and CD tissues, none of the active UC-associated miRNAs were differentially expressed when compared with healthy control tissues.

### Example 3

### In Situ Hybridization Localization of miR-192

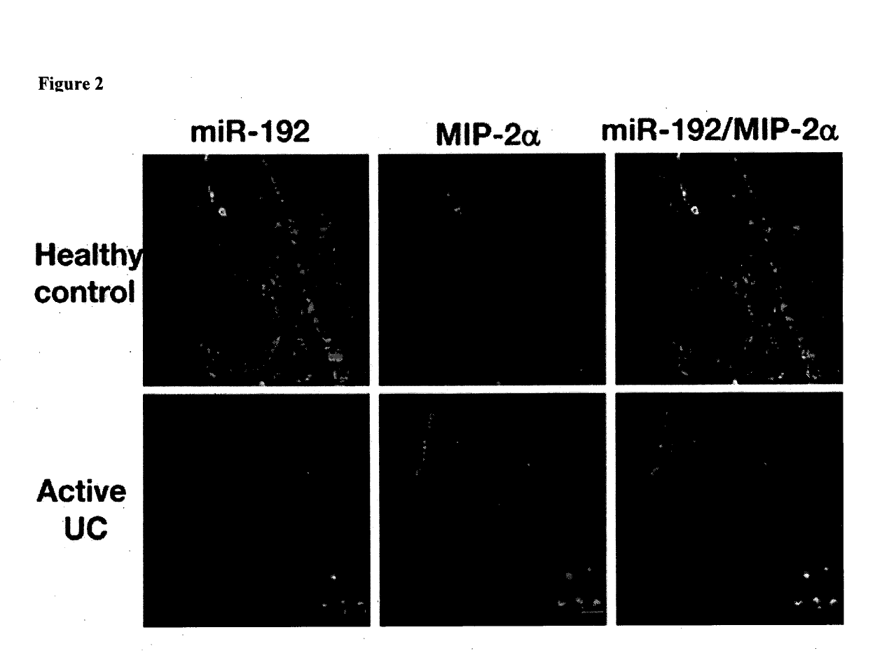

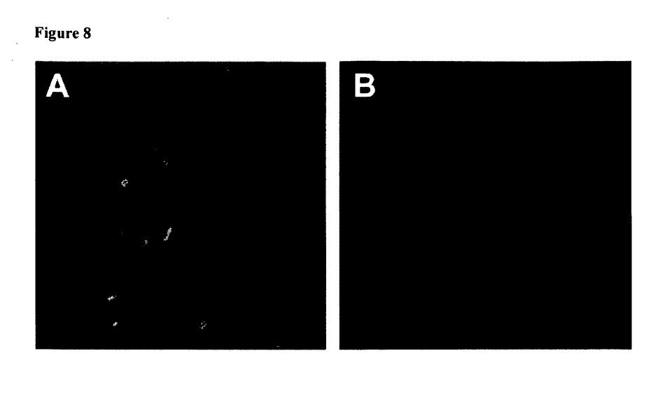

Given the abundant expression of miR-192 in the miRNA microarray analysis and subsequent qRT-PCR, it was next determined which cell types express miR-192 by performing in situ hybridization on colon biopsy samples. The expression of miR-192 was found to be predominantly expressed in epithelial cells in the normal colon. In active UC tissues, miR-192 expression in the epithelial layer appeared qualitatively decreased (FIG. 2). The negative in situ hybridization controls demonstrated fluorescence in scattered lamina propria cells but no fluorescence in epithelial cells (FIG. 8).

### Example 4

### mRNA Microarray Analysis of Active UC Tissues to Identify Potential miRNA Targets

Target genes containing binding sites for the 8 active UC-associated miRNAs were expected to demonstrate altered expression levels in active UC tissues. To identify potential targets of the active UC-associated miRNAs, a human genome-wide mRNA microarray was used to screen pooled large RNAs from biopsy samples of patients with active UC and healthy control subjects. Overall, 876 genes were increased in active UC patients and 267 genes were decreased in active UC patients (Table 8).

Because miR-192 was localized to colonic epithelial cells and its expression was decreased in active UC, target genes expressed by colonic epithelial cells were focused upon. Among the genes that were increased in active UC tissues were 12 colonic epithelial-derived cytokines and chemokines. These cytokines and chemokines included monocyte chemoattractant protein-1 (CCL2), MIP-1α (CCL3), MIP-1β (CCL4), MIP-3α (CCL20), GRO1 (CXCL1), MIP-2α (CXCL2), GRO3 (CXCL3), epithelial neutrophil-activating peptide-78 (CXCL5), granulocyte chemoattractant protein-2 (CXCL6), interferon-inducible protein-10 (CXCL10), IL-8, and IL-18 (Table 8). The mRNA sequences of these cytokines and chemokines were subsequently analyzed for putative miRNA binding sites (available on the world wide web at microrna.sanger.ac.uk/targets/v3/; Griffiths-Jones et al. (2006) Nucleic Acids Res 0.34, D140-D144).

The MIP-2α mRNA was found to contain putative binding sites for 9 miRNAs in its 3′UTR (Table 2). Four miRNAs—miR-27b, miR-603, miR-532 and miR-769-5p—were predicted to have unique binding sites on the MIP-2α mRNA. In addition, miR-141, miR-200a, and miR-217 share an overlapping binding site on the MIP-2α mRNA. Similarly, miR-192 and miR-215 share an overlapping binding site on the MIP-2α mRNA.

According to the microarray analysis of human tissues, miR-192 was the most abundant of the 9 miRNAs with putative binding sites on the MIP-2α mRNA and the only miRNA that was differentially expressed in active UC tissues. Furthermore, whereas miR-192 and miR-215 share a common binding site on the MIP-2α mRNA, qRT-PCR analysis demonstrated that the expression of miR-192 in healthy control tissues was 16.0 times more abundant than miR-215 and confirmed that miR-215 expression was unchanged in active UC tissues.

### Example 5

### MIP-2α and miR-192 Expression are Inversely Correlated in Human Tissues

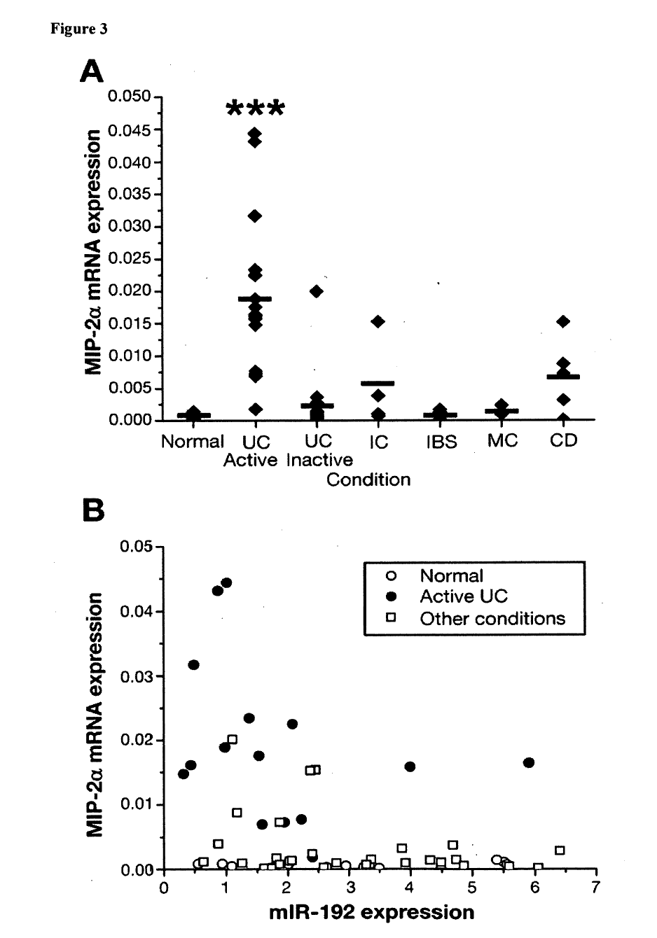

MIP-2α, a chemotactic cytokine produced by colonic epithelial cells and macrophages (Ohtsuka et al. (2001) Gut 49, 526-533; Wolpe et al. (1989) Proc. Natl. Acad. Sci. U.S.A 86, 612-616) was identified in the microarray analysis to be significantly increased in active UC tissues and confirmed by qRT-PCR analysis (FIG. 3A). A 32.2-fold increase in MIP-2α mRNA expression was observed in active UC tissues relative to healthy control tissues (P<0.001; FIG. 3A). When compared with healthy control tissues, the expression of MIP-2α was not statistically different in patients with inactive UC, IC, MC, IBS, and CD.

A comparison of MIP-2α mRNA expression with miR-192 in all 60 human biopsy tissues demonstrated that they are inversely correlated (FIG. 3B; r=−0.325; P<0.01). Furthermore, combined immunohistochemistry and in situ hybridization was conducted on biopsy samples for MIP-2α and miR-192, respectively (FIG. 2). MIP-2α protein was localized to epithelial cells and scattered lamina propria mononuclear cells in active UC tissues but not detected in epithelial cells in healthy control colon tissues, indicating that MIP-2α and miR-192 are expressed in similar cell types in the human colon but under opposing conditions. The negative immunohistochemistry control demonstrated no staining in colonic epithelial cells (FIG. 8).

### Example 6

### Regulation of MIP-2α by miRNAs in Colonic Epithelial Cells

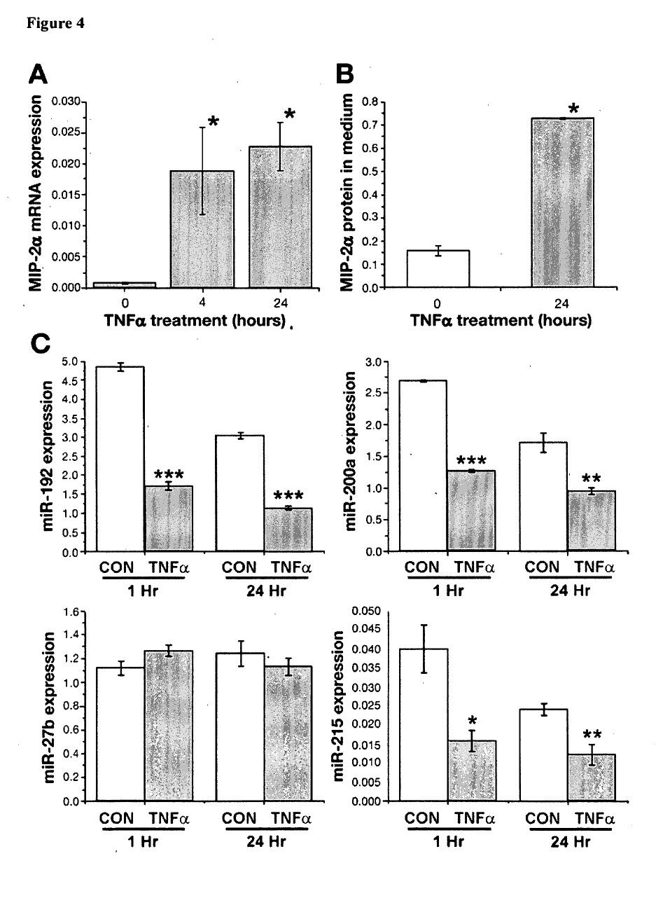

The in vivo data indicated a negative correlation between MIP-2α and miR-192 expression in active UC tissues, raising the possibility that inflammatory mediators may differentially regulate MIP-2α and miR-192 expression. This negative correlation was tested for in an in vitro model, using inflammatory cytokine-stimulated colonic epithelial cells. HT29 cells stimulated with TNF-α resulted in a 137-fold and a 166-fold increase in MIP-2α mRNA expression at 1 and 24 hours, respectively (FIG. 4A; P<0.05). Similarly, TNF-α induced a 4.6-fold increase in MIP-2α protein secretion at 24 hours (FIG. 4B; P<0.05).

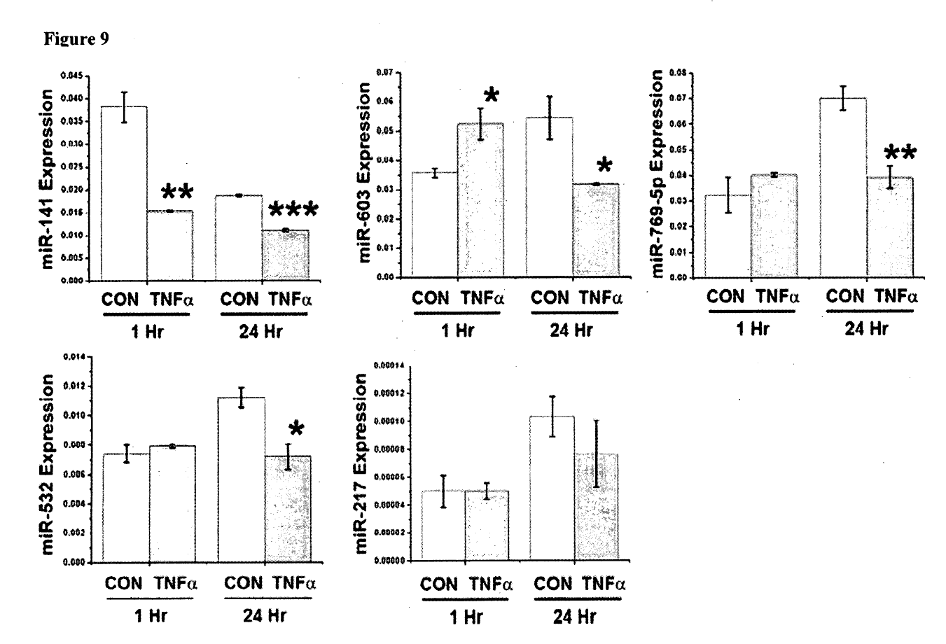

This MIP-2α induction was accompanied by TNF-α-induced alterations in several of the miRNAs with putative binding sites on the MIP-2α mRNA (FIG. 4C and FIG. 9). Specifically, the expression of miR-192, miR-215, miR-141, and miR-200a were all significantly decreased in TNF-α-stimulated HT29 cells at 1 and 24 hours. Significantly less miR-532, miR-603, and miR-769-5p was observed 24 hours after TNF-α stimulation as compared with unstimulated cells. The expression of miR-27b and miR-217 were not significantly altered with TNF-α stimulation.

Based on the data demonstrating inverse patterns of TNF-α-induced MIP-2α and associated miRNA expression, it was hypothesized that several of these putative miRNAs may regulate MIP-2α production. To determine which endogenous miRNAs may influence MIP-2α expression, a luciferase reporter construct containing the MIP-2α 3′UTR was generated and transfected into unstimulated cells. Seven additional luciferase reporter constructs containing mutations in each of the putative miRNA binding sites were generated (FIGS. 5A and 5B).

Transfecting the pMIR reporter construct containing the wild-type MIP-2α 3′UTR into HT29 cells resulted in a 39.0% reduction in luciferase activity (FIG. 5C), indicating that the MIP-2α 3′UTR and endogenous miRNAs can influence MIP-2α gene expression (P<0.001). Mutating the miR-192/miR-215 binding site in the MIP-2α 3′UTR resulted in a restoration of luciferase activity to 73% of the original pMIR reporter (P<0.05). Mutating the binding sites for miR-603 and miR-141/200a resulted in a restoration of luciferase activity to 61.1% and 60.1% of the original pMIR reporter, respectively (P<0.05). Mutations in the putative binding sites for miR-27b, miR-532, miR-217, and miR-769-5p did not significantly influence reporter activity. Results indicate that 3 miRNA binding sites, miR-192/215, miR-141/200a, and miR-603, influence MIP-2α expression in HT29 cells.

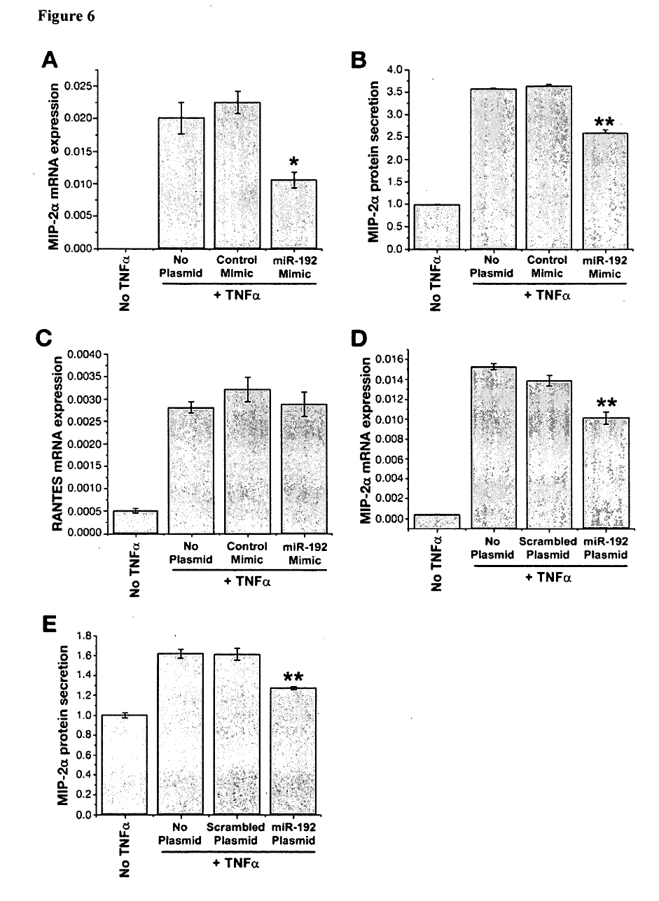

Whether miR-192 can negatively influence inducible MIP-2α expression in an ex vivo cell culture system was also test. An miR-192 mimic or an miR-192 overexpression construct was transiently transfected into HT29 cells and TNF-α-induced MIP-2α mRNA expression and protein secretion assessed (FIG. 6). By 4 hours of TNF-α stimulation, MIP-2α mRNA expression in cells transfected with the miR-192 mimic was decreased by 53% (FIG. 6A). Similarly, the miR-192 mimic reduced secreted MIP-2α protein by 28% after 24 hours of TNF-α stimulation (FIG. 6B). The control mimic did not significantly reduce either MIP-2α mRNA expression or protein secretion. Similarly, the transfection of an miR-192 overexpression construct into HT29 cells reduced the TNF-α-stimulated MIP-2α mRNA expression and protein secretion by 34% and 21%, respectively (FIGS. 6D and 6E). The control plasmid containing a scrambled miR-192 sequence did not significantly reduce either MIP-2α mRNA expression or protein secretion. Furthermore, the miR-192 mimic did not inhibit the TNF-α-stimulated expression of RANTES (FIG. 6C), a TNF-α-stimulated epithelial chemokine that does not contain a putative miR-192 binding site. The results indicate that the effect of miR-192 on the TNF-α-induced expression of MIP-2α was not due to a global effect on cytokine production.

Accordingly, these results demonstrate that miRNAs are differentially expressed in the tissues of patients with active UC as compared with normal, healthy control subjects. Specifically, 8 up-regulated and 3 down-regulated miRNAs were identified in active UC tissues. The pattern of expression of these active UC-associated miRNAs was distinct from other conditions, including inactive UC, IC, MC, CD, and IBS. This study is the first to link chronic IBD with altered expression of miRNAs, thereby expanding the known inflammatory diseases associated with miRNAs, which previously included chronic pancreatitis and hepatitis (Bloomston et al. (2007) JAMA 297, 1901-1908; Murakami et al. (2006) Oncogene 25, 2537-2545).

The results herein demonstrate miR-192 expression in colonic epithelial cells, a significant reduction in expression in active UC tissues, and an inverse correlation between the expression of mir-192 and MIP-2α, an epithelial cell-expressed chemokine previously implicated in IBD and murine colitis (Dieckgraefe et al. (2000) Physiol. Genomics 4, 1-11; Lawrance et al. (2001) Hum. Mol. Gen. 10, 445-456; Wu et al. (2007) Inflamm. Bowel Dis. 13, 807-821; Dooley et al. (2004) Inflamm. Bowel Dis. 10, 1-14; Ohtsuka and Sanderson (2003) Pediatr. Res. 53, 143-147). In colonic epithelial cells, basal levels of miR-192 can regulate MIP-2α expression. In the same colonic epithelial cells, TNF-α-induced MIP-2α expression was also influenced by miR-192. These findings, demonstrating its regulation of epithelial chemokine expression, expand the known roles of miR-192 to include the regulation of long-lived chemokines

Previously, miR-192 was shown to be induced by transforming growth factor (TGF)-β and regulate Smad-interacting protein 1 expression in murine mesangial cells, thereby implicating it in collagen regulation and diabetic nephropathy (Kato et al. (2007) Proc. Natl. Acad. Sci. U.S.A. 104, 3432-3437). Its regulation by TGF-β and TNF-α and its regulation of collagen expression and chemokine expression indicate that miR-192 may play a key role in processes of inflammation and fibrosis. This is reflected in the demonstration that other putative miR-192 targets include components of microbial response pathways and other inflammatory and fibrosis mediators, including NOD2, Toll-like receptor 6, TRAF-interacting protein, CC chemokine receptor 6, IL-18 receptor, and matrix metalloproteinase 16. Further studies are necessary to determine whether miR-192 regulates each of these putative targets and influences models of innate immunity, inflammation, and fibrosis.

Of the other 10 active UC-associated miRNAs, previous studies indicate a potential role for 3 of these miRNAs in inflammation: miR-21, miR-16, and Let-7f were previously found to be increased in T-cell subtypes (Wu et al. (2007) PLoS ONE 2, e1020). In addition, miR-21 was increased in the lungs of mice exposed to aerosolized lipopolysaccharide (Moschos et al. (2007) BMC Genomics 8, 240) and miR-16 was shown to influence the degradation of mRNAs containing the AU-rich elements from the TNF, IL-8, and IL-6 3′UTRs (Jing et al. (2005) Cell 120, 623-634). The identification of other targets of the active UC-associated miRNAs, the confirmation of their regulation by miRNAs and the examination of the influence of these miRNAs on intestinal inflammation in experimental models of colitis will likely be the subject of future studies.

It is notable that 11 miRNAs were found to be differentially expressed in patients with active UC as compared with healthy control subjects. However, a similar expression pattern of these miRNAs was not seen in inactive UC tissues. In inactive UC tissues, only 3 miRNAs demonstrated a similar expression pattern to that seen in active UC. The expression of 2 miRNAs was opposite to that seen with active UC; 6 miRNAs were unchanged in inactive UC tissues. This indicates that individual miRNAs may influence varying aspects of inflammation, including acute and chronic inflammation. Similarly, none of the active UC-associated miRNAs were differentially expressed in the active Crohn's colitis or MC tissues. Although it is highly likely that there exist miRNAs that are differentially expressed in active Crohn's colitis and MC, the findings support the likelihood that the pathogenesis of these subtypes of IBD are distinct from active UC. Further studies are necessary to identify the miRNAs associated with MC and Crohn's colitis as well as CD involving the small intestine. Furthermore, because evidence indicates that CD and UC are characterized by differing Th1 and Th2 cytokine profiles, it will be interesting to examine the role of miRNAs in modulating Th1 and Th2 responses. These results support miRNAs as key negative regulators of inflammation. A further understanding of the regulation and role of miRNAs in acute and chronic inflammatory diseases may lead to the use of miRNAs in the diagnosis and the use of miRNA mimics and inhibitors in the treatment of chronic inflammatory diseases.

### Example 7

### Assessment of Peripheral Blood miRNA Expression in Active IBD

In order to demonstrate the applicability of miRNA profiling for the assessment of IBD to diverse subject samples, peripheral blood, serum, and plasmid miRNA extraction and expression were compared. A stable miRNA extraction protocol was developed from peripheral blood. Briefly, a total of 2.5 cc of blood was collected into PAXgene™ tubes and centrifuged to remove lysed blood cells using the PAXgene™ Blood RNA Kit (PreAnalytiX). After washing, total RNA was extracted using the miRNeasy mini kit (Qiagen). The RediPlate 96 Ribo Green RNA Quantitation Kit (Invitrogen) was used to quantitate the RNA molecules in each sample. The RNA samples were stored at −80° C. Our laboratory has found that each PAXgene™ tube yields between 2.4 to 8 μg of total RNA. Total RNA was labeled and microRNA expression assessed using the multi-species miRNA microarray (Dharmacon). The raw hybridization intensities were obtained after arrays scanned. Data normalization and analysis were performed using the Dharmacon proprietary software specified for microRNA microarray. Findings were considered significant if a greater than two-fold difference is observed when comparing the mean hybridization intensities of blood with other intestinal conditions and normal, healthy controls (Tables 11 and 12).

### Example 8

### Assessment of miRNA Expression in Crohn's Disease Tissues

Sigmoid colon biopsy samples from five CD patients and fifteen normal controls were used to generate miRNA microarray profiles using microRNA microarray. Terminal ileal biopsy samples from seven CD patients and seven normal controls were used to generate miRNA microarray profiles. All results were validated using qRT-PCR. The results comparing sigmoid colon microRNA expression in CD patients as compared to normal controls are shown in Table 9. Overall, one microRNA was increased in sigmoid colon tissues of CD patients while 11 were decreased. The results comparing terminal ileal microRNA expression in CD patients as compared to normal controls are shown in Table 10. Overall, seven microRNAs were found to be differentially expressed in the terminal ileum of CD patients.

### Example 9

### Assessment of miRNAs in Murine Colitis

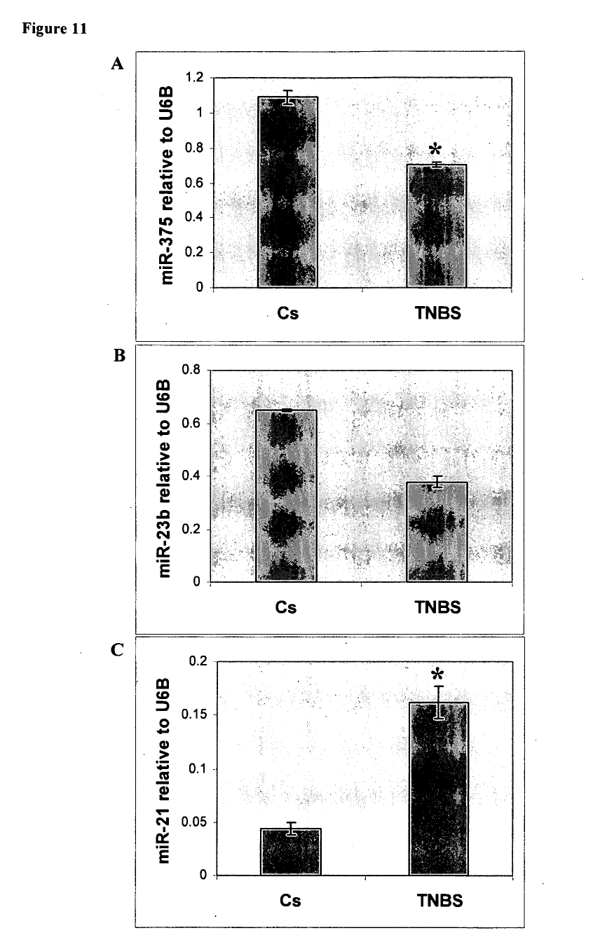

Differentially expressed miRNAs were identified in a murine model of colitis (i.e., trinitrobenzene sulfuric acid (TNBS)-induced colitis). It was determined that 23 miRNAs were downregulated, whereas 4 miRNAs were upregulated (Table 13). Several TNBS-associated miRNAs are differentially expressed in active UC, as shown in FIG. 11. Thus, miRNAs are differentially expressed in the colons of mice with TNBS-induced colitis and the miRNA profile in TNBS-induced colitis are similar to that of human IBD.

### Example 10

### Delivery of miRNA Mimics and Inhibitors In Vivo

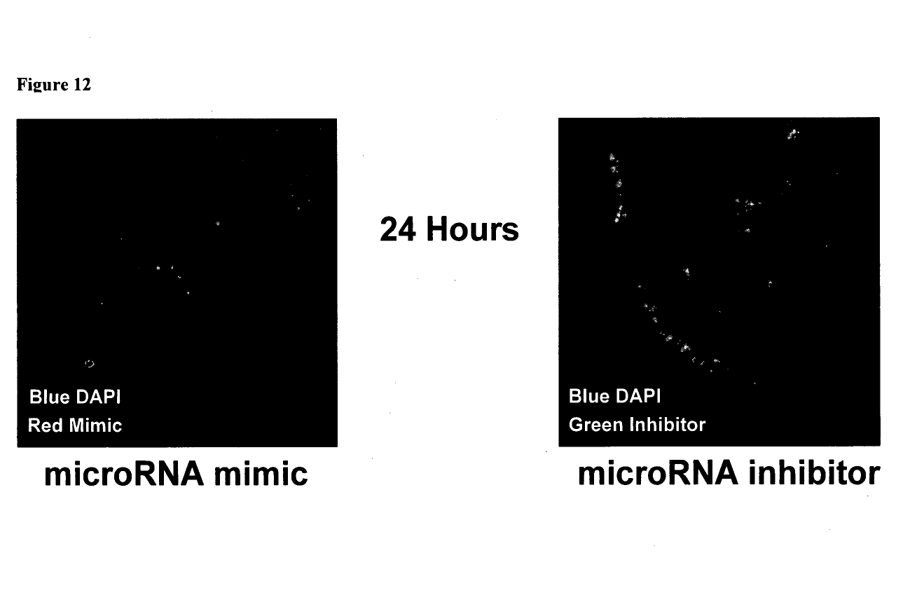

miRNA mimics and inhibitors were designed and tested for delivery into colonic tissue in vivo. In particular, peri-rectal and tail vein injection of miRNA mimics and inhibitors were assessed in a mice. Briefly, a 5′-DY547-labeled double-stranded RNA oligonucleotide microRNA control mimic (Dharmacon) and a 3′-6FAM-labeled DNA oligonucleotide (Exiqon) were designed. Six to eight week old C57BL6 mice were anesthetized with 0.25 ml Avertin (Tribromoethanol 12.5 mg/ml) i.p. and varying concentrations of microRNA mimics or inhibitors (in 0.1 ml) instilled per rectum using a 20G mouse feeding needle inserted 3-4 cm from the anus. Colon tissues from mice were snap frozen in OCT media at 4 hrs, 24 hours and 72 hours. Cryosections (5-6 μm) were fixed in 4% paraformaldehyde in PBS at 4° C. for 10 min. Sections were counterstained with DAPI for 1 min and mounted. Immunofluorescence images will be captured with a Zeiss LSM 410 confocal microscope using Metamorph and Velocity software, in conjunction with the Hopkins Digestive Diseases Basic Research Development Core. FIG. 12 indicates successful delivery of such agents to the desired tissues and specific epithelial cell uptake.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org, the National Center for Biotechnology Information (NCBI) on the world wide web at ncbi.nlm.nih.gov, or miRBase on the world wide web at microrna.sanger.ac.uk.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1
Clinical Characteristics of Patients

| | | Active | Inactive | | | | Crohn's |
|---|---|---|---|---|---|---|---|
| | Control | UC | UC | IC | IBS | MC | disease |
| No. of patients | 15 | 15 | 15 | 4 | 5 | 3 | 5 |
| Male, n (%) | 6 (40) | 7 (46.7) | 7 (46.7) | 1 (25) | 0 (0) | 0 (0) | 3 (60) |

Age (y)

| | | | | | | | |
|---|---|---|---|---|---|---|---|
| Mean | 53.3 | 35.3 | 41.1 | 55 | 37.4 | 42 | 32.6 |
| Range | 38-68 | 18-55 | 23-61 | 33-72 | 26-54 | 27-63 | 23-51 |

Duration of IBD (y)

| | | | | | | | |
|---|---|---|---|---|---|---|---|
| Mean | | 6.2 | 12.4 | | | 3.2 | 10.2 |
| Range | NA | 0.25-26 | 1-29 | NA | NA | 1.5-6 | 1-22 |
| Medications | | | | | | | |
| Mesalamine, n (%) | 0 | 12 (80) | 15 (100) | 0 | 0 | 1 (33) | 3 (60) |
| Antibiotics, n (%) | 0 | 2 (13.3) | 0 | 2 (50) | 0 | 0 | 1 (20) |
| Steroids, n (%) | 0 | 4 (27) | 2 (13.3) | 0 | 0 | 0 | 0 |
| Immunomodulators, | 0 | 8 (53) | 7 (46.7) | 0 | 0 | 1 (33) | 0 |
| n (%) | | | | | | | |
| Biologics, n (%) | 0 | 0 | 1 (6.7) | 0 | 0 | 0 | 1 (20) |

IBD, inflammatory bowel disease;
IBS, irritable bowel syndrome;
IC, infectious colitis;
MC, microscopic colitis;
UC, ulcerative colitis

TABLE 2
miRNAs With Binding Sites in Macrophage Inflammatory Peptide-2α
and Their miRNA Microarray Hybridization Intensities in Tissues

| | MicroRNA | | | |
|---|---|---|---|---|
| | binding site | Healthy | Active | Inactive |
| MicroRNA | position | control | UC | UC |
| miR-192 | 741-758 | 1190 ± 126 | 676 ± 93* | 1225 ± 86 |
| miR-215 | 741-758 | 354 ± 90 | 257 ± 62 | 353 ± 93 |
| miR-27b | 857-875 | 61 ± 15 | 95 ± 8 | 127 ± 15 |
| miR-603 | 985-1005 | 14 ± 1 | 12 ± 1 | 14 ± 1 |
| miR-532 | 1071-1008 | 18 ± 3 | 18 ± 3 | 22 ± 2 |
| miR-217 | 1097-1118 | 12 ± 2 | 12 ± 1 | 13 ± 1 |
| miR-200a | 1100-1121 | 69 ± 12 | 76 ± 9 | 116 ± 14 |
| miR-141 | 1102-1121 | 31 ± 5 | 23 ± 3 | 42 ± 4 |
| miR-769- | 1131-1149 | 11 ± 2 | 9 ± 1 | 9 ± 1 |
| 5p | | | | |

TABLE 3
Primers Used for Quantitative Real-Time Polymerase
Chain Reactions (PCR)

| | |
|---|---|
| Name | Primer (5′-3′) |

For microRNA

| | | |
|---|---|---|
| Universal | Reverse | NCode™ miRNA First-strand |
| quantitative | | cDNA synthesis kits |
| PCR primer | | (Invitrogen) |
| Let-7a | Forward | tgaggtagtaggttgtatagtt |
| Let-7f | Forward | tgaggtagtagattgtatagtt |
| miR-126 | Forward | tcgtaccgtgagtaataatgc |
| miR-16 | Forward | tagcagcacgtaaatattggcg |
| miR-19b | Forward | tgtgcaaatccatgcaaaactga |
| miR-192 | Forward | ctgacctatgaattgacagcc |
| miR-195 | Forward | tagcagcacagaaatattggc |
| miR-199a* | Forward | tacagtagtctgcacattggtt |
| miR-21 | Forward | tagcttatcagactgatgttga |
| miR-203 | Forward | gtgaaatgtttaggaccactag |
| miR-215 | Forward | atgacctatgaattgacagac |
| miR-23a | Forward | atcacattgccagggatttcc |
| miR-23b | Forward | atcacattgccagggattacc |
| miR-24 | Forward | tggctcagttcagcaggaacag |
| miR-26a | Forward | ttcaagtaatccaggataggc |
| miR-29a | Forward | tagcaccatctgaaatcggtt |
| miR-320 | Forward | aaaagctgggttgagagggcgaa |
| miR-375 | Forward | tttgttcgttcggctcgcgtga |
| miR-422b (also | Forward | ctggacttggagtcagaaggcc |
| known as miR-378) | | |
| miR-629 | Forward | gttctcccaacgtaagcccagc |
| miR-141 | Forward | taacactgtctggtaaagatgg |
| miR-27b | Forward | ttcacagtggctaagttctgc |
| miR-603 | Forward | cacacactgcaattacttttgc |
| miR-532 | Forward | catgccttgagtgtaggaccgt |
| miR-217 | Forward | tactgcatcaggaactgattggat |
| miR-200a | Forward | taacactgtctggtaacgatgt |
| miR-769-5p | Forward | tgagacctctgggttctgagct |
| U6a | Forward | ctcgcttcggcagcaca |

For mRNA

| | | |
|---|---|---|
| GAPDH | Forward | GTCTCCTCTGACTTCAACA |
| | Reverse | CAGGAAATGAGCTTGACAAA |
| MIP2A (CXCL2) | Forward | CTCAAGAATGGGCAGAAAGC |
| | Reverse | CTTCAGGAACAGCCACCAAT |
| GRO1 (CXCL1) | Forward | CCAAAGTGTGAACGTGAAG |
| | Reverse | TGGGGGATGCAGGATTGA |
| MIP2A 3′UTR | Forward | TCTACTTGCACACTCTCCCATT |
| | Reverse | GCCTCTATCACAGTGGCTGA |

TABLE 4
miRNAs Differentially Expressed in Active Ulcerative Colitis (UC)
Tissues as Compared With Normal, Healthy Controls

| miRNA | Normal | Active UC | Fold change |
|---|---|---|---|
| miR-19b | 334 ± 92 | 79 ± 8 | −4.6 |
| miR-192 | 1190 ± 126 | 676 ± 93 | −1.8 |
| miR-320 | 650 ± 142 | 147 ± 21 | −4.4 |
| miR-375 | 457 ± 76 | 172 ± 24 | −2.7 |
| miR-422b | 539 ± 133 | 42 ± 6 | −12.8 |
| (also known | | | |
| as miR-378) | | | |
| miR-629 | 364 ± 103 | 95 ± 23 | −3.8 |
| miR-16 | 371 ± 98 | 971 ± 86 | 2.6 |
| miR-21 | 117 ± 25 | 912 ± 156 | 7.8 |
| miR-23a | 177 ± 39 | 519 ± 79 | 2.9 |
| miR-23b | 112 ± 26 | 289 ± 37 | 2.6 |
| miR-24 | 320 ± 57 | 934 ± 89 | 2.9 |
| miR-26a | 564 ± 122 | 1032 ± 63 | 1.8 |
| miR-29a | 159 ± 33 | 470 ± 81 | 3.0 |
| miR-126 | 114 ± 24 | 438 ± 85 | 3.9 |
| miR-195 | 122 ± 27 | 235 ± 19 | 1.9 |
| miR-199a* | 107 ± 23 | 269 ± 33 | 2.5 |
| miR-203 | 97 ± 23 | 279 ± 55 | 2.9 |
| Let-7f | 163 ± 40 | 476 ± 83 | 2.9 |

TABLE 5
miRNAs Differentially Expressed in Inactive Ulcerative Colitis (UC)
Tissues as Compared With Normal, Healthy Controls

| miRNA | Normal | Inactive UC | Fold change |
|---|---|---|---|
| miR-19b | 334 ± 92 | 94 ± 11 | −3.9 |
| miR-422b | 539 ± 133 | 110 ± 16 | −4.9 |
| (also known | | | |
| as miR-378) | | | |
| miR-629 | 364 ± 103 | 92 ± 53 | −3.9 |
| miR-21 | 117 ± 25 | 249 ± 46 | 2.1 |
| miR-23a | 177 ± 39 | 409 ± 49 | 2.3 |
| miR-23b | 112 ± 26 | 303 ± 28 | 2.7 |
| miR-26a | 564 ± 122 | 1039 ± 54 | 1.8 |
| miR-29a | 159 ± 33 | 329 ± 32 | 2.1 |
| miR-126 | 114 ± 24 | 233 ± 39 | 2.1 |
| miR-195 | 122 ± 27 | 247 ± 26 | 2.0 |
| miR-199a | 107 ± 23 | 219 ± 21 | 2.0 |
| Let-7f | 163 ± 40 | 302 ± 45 | 1.9 |

TABLE 6
miRNAs Differentially Expressed in Active Ulcerative Colitis (UC)
Tissues as Compared With Inactive UC Tissues

| miRNA | Active UC | Inactive UC | Fold Change |
|---|---|---|---|
| miR-16 | 971 ± 86 | 556 ± 68 | −1.7 |
| miR-21 | 912 ± 156 | 249 ± 46 | −3.7 |
| miR-24 | 934 ± 89 | 451 ± 56 | −2.1 |
| miR-126 | 438 ± 85 | 233 ± 39 | −1.9 |
| miR-203 | 279 ± 55 | 134 ± 18 | −2.1 |
| miR-200b | 291 ± 33 | 613 ± 79 | 2.1 |

TABLE 7
Relative Quantitative Reverse Transcription-Polymerase Chain Reaction Expression
Levels of Active Ulcerative Colitis (UC)-Associated miRNAs

| Micro | Normal | Active | Inactive | | | | Crohn's |
|---|---|---|---|---|---|---|---|
| RNA | Control | UC | UC | IC | IBS | MC | Disease |
| miR- | 3.424 ± 0.440 | 1.813 ± 0.216^{b} | 3.733 ± 0.225 | 2.425 ± 0.412 | 2.967 ± 0.442 | 1.437 ± 0.261 | 2.206 ± 0.250 |
| 192 | | | | | | | |
| miR- | 0.581 ± 0.047 | 0.229 ± 0.039^{a} | 1.402 ± 0.102^{c} | 1.276 ± 0.151^{c} | 1.229 ± 0.198^{c} | 0.522 ± 0.118 | 0.893 ± 0.104 |
| 375 | | | | | | | |
| miR- | 0.0589 ± 0.006 | 0.018 ± 0.003^{c} | 0.109 ± 0.008^{c} | 0.108 ± 0.021^{a} | 0.110 ± 0.012^{c} | 0.050 ± 0.009 | 0.063 ± 0.005 |
| 422b | | | | | | | |
| miR- | 5.478 ± 0.504 | 19.427 ± 0.939^{c} | 5.290 ± 0.320 | 9.073 ± 0.581 | 4.129 ± 0.462 | 7.011 ± 1.386 | 7.123 ± 0.624 |
| 21 | | | | | | | |
| miR- | 0.475 ± 0.057 | 1.846 ± 0.128^{c} | 0.455 ± 0.032 | 0.437 ± 0.044 | 0.495 ± 0.060 | 0.620 ± 0.099 | 0.534 ± 0.037 |
| 126 | | | | | | | |
| miR- | 0.431 ± 0.057 | 1.068 ± 0.093^{c} | 0.690 ± 0.041 | 0.616 ± 0.062 | 0.748 ± 0.074 | 0.815 ± 0.83 | 0.660 ± 0.063 |
| 195 | | | | | | | |
| miR- | 0.419 ± 0.050 | 0.844 ± 0.080^{c} | 0.885 ± 0.057^{c} | 0.853 ± 0.052^{b} | 0.882 ± 0.082^{a} | 0.834 ± 0.025 | 0.714 ± 0.030 |
| 23a | | | | | | | |
| miR- | 0.368 ± 0.049 | 0.824 ± 0.076^{c} | 0.669 ± 0.041^{c} | 0.601 ± 0.063 | 0.626 ± 0.049 | 0.706 ± 0.101 | 0.635 ± 0.058 |
| 16 | | | | | | | |
| Let-7f | 0.489 ± 0.047 | 0.757 ± 0.040^{c} | 0.415 ± 0.019 | 0.382 ± 0.031 | 0.379 ± 0.033 | 0.319 ± 0.017 | 0.476 ± 0.044 |
| miR- | 0.082 ± 0.010 | 0.173 ± 0.020^{c} | 0.242 ± 0.015^{c} | 0.155 ± 0.010 | 0.165 ± 0.015 | 0.178 ± 0.015 | 0.132 ± 0.014 |
| 24 | | | | | | | |
| miR- | 0.037 ± 0.003 | 0.069 ± 0.009^{a} | 0.138 ± 0.009^{c} | 0.053 ± 0.007 | 0.051 ± 0.004 | 0.412 ± 0.130 | 0.043 ± 0.005 |
| 29a | | | | | | | |

TABLE 8
Genes Differentially Expressed in Ulcerative Colitis (UC) Patients Versus Normal
Controls

| | | Hybridization | |
|---|---|---|---|
| | | intensity (Log₂) | |
| Gene | Accession # | Normal | UC |

Up-regulated in UC

| | | | |
|---|---|---|---|
| 6-phosphofructo-2-kinase/fructose-2,6- | NM_004566 | 8.06 | 10.16 |
| biphosphatase 3 | | | |
| a disintegrin and metalloproteinase domain 9 | NM_003816 | 9.16 | 10.72 |
| (meltrin γ) | | | |
| a disintegrin-like and metalloprotease (reprolysin | NM_007038 | 6.25 | 7.46 |
| type) with thrombospondin type 1 motif, 5 | | | |
| (aggrecanase-2) | | | |
| a disintegrin-like and metalloprotease (reprolysin | AI431730 | 5.23 | 8.27 |
| type) with thrombospondin type 1 motif, 9 | | | |
| Abhydrolase domain containing 2 | NM_007011 | 6.3 | 7.32 |
| ABI gene family, member 3 (NESH) binding | NM_024801 | 5.51 | 7.33 |
| protein | | | |
| Absent in melanoma 2 | NM_004833 | 7.26 | 8.29 |
| Actinin, α 1 | AI082078 | 9.46 | 10.48 |
| Activating signal cointegrator 1 complex subunit 3 | AA156961 | 7.74 | 8.79 |
| Activin A receptor, type I | NM_001105 | 7.77 | 8.8 |
| Acyl-CoA synthetase long-chain family member 1 | NM_021122 | 7.45 | 8.86 |
| Acyl-CoA synthetase long-chain family member 3 | BF512846 | 6.43 | 7.44 |
| Acyl-CoA synthetase long-chain family member 4 | NM_022977 | 7.58 | 9.41 |
| Adenosine deaminase | NM_000022 | 5.44 | 7.1 |
| Adipocyte-specific adhesion molecule | BG112263 | 7.71 | 9.16 |
| Adlican | AF245505 | 8.65 | 10.29 |
| ADP-ribosylation factor GTPase activating | BC005122 | 8.47 | 10.12 |
| protein 3 | | | |
| ADP-ribosylation factor-like 3 | NM_004311 | 7.44 | 8.51 |
| ADP-ribosylation factor-like 7 | BG435404 | 6.76 | 8.13 |
| Adrenomedullin | NM_001124 | 9.09 | 10.94 |
| AE binding protein 1 | NM_001129 | 6.65 | 7.72 |
| AER61 glycosyltransferase | AK023140 | 6.15 | 7.2 |
| Alcohol dehydrogenase IB (class I), β | AF153821 | 5.51 | 6.69 |
| polypeptide ? | | | |
| Aldehyde dehydrogenase 1 family, member A2 | AB015228 | 6.04 | 7.32 |
| Aldolase B, fructose-bisphosphate | AK026411 | 4.77 | 9.41 |
| Allograft inflammatory factor 1 | U19713 | 7.83 | 8.97 |
| α-2-Glycoprotein 1, zinc | D90427 | 5.99 | 7.23 |
| Amyloid β (A4) precursor protein-binding, family | AI093231 | 6.43 | 8.02 |
| B, member 1 interacting protein | | | |
| Angiopoietin-like 2 | AF007150 | 6.65 | 8.3 |
| Angiopoietin-like 4 | NM_016109 | 7.21 | 8.22 |
| Angiotensin II receptor-like 1 | X89271 | 6.38 | 7.89 |
| Ankyrin repeat domain 22 | AI925518 | 7.3 | 9.23 |
| Ankyrin repeat domain 28 | N32051 | 7.56 | 8.64 |
| Annexin A1 | NM_000700 | 8.85 | 11.92 |
| Annexin A10 | AF196478 | 5.66 | 9.18 |
| Annexin A3 | M63310 | 8.28 | 9.84 |
| Annexin A5 | NM_001154 | 9.88 | 10.94 |
| Annexin A6 | NM_001155 | 8.17 | 9.26 |
| Anterior gradient 2 homolog (Xenopus laevis) | AI922323 | 10.76 | 11.8 |
| Apolipoprotein B mRNA editing enzyme, catalytic | NM_001644 | 7.1 | 9.28 |
| polypeptide 1 | | | |
| Apolipoprotein B mRNA editing enzyme, catalytic | NM_021822 | 6.74 | 7.83 |
| polypeptide-like 3G | | | |
| Apolipoprotein C-IV | NM_001646 | 4.89 | 6.98 |
| Apolipoprotein L, 1 | AF323540 | 6.77 | 9.72 |
| Apolipoprotein L, 2 | BC004395 | 5.72 | 7.43 |
| Apolipoprotein L, 3 | NM_014349 | 7 | 8.06 |
| Apoptosis inhibitor 5 | AF229253 | 6.04 | 7.06 |
| Aquaporin 9 | NM_020980 | 3.46 | 8.19 |
| Arachidonate 5-lipoxygenase | NM_000698 | 7.25 | 8.83 |
| Arachidonate 5-lipoxygenase-activating protein | NM_001629 | 7.55 | 8.61 |
| ARG99 protein | AU151239 | 7.5 | 8.97 |
| Argininosuccinate synthetase | NM_000050 | 10.56 | 11.98 |
| Aryl hydrocarbon receptor | NM_001621 | 8.4 | 9.6 |
| Aryl hydrocarbon receptor nuclear translocator- | AF256215 | 5.55 | 7.21 |
| like 2 | | | |
| asp (abnormal spindle)-like, microcephaly | AK001380 | 5.87 | 6.95 |
| associated (Drosophila) | | | |
| Aspartate β-hydroxylase | AF289489 | 10.16 | 11.23 |
| ATPase, class V, type 10D | AI478147 | 6.23 | 7.26 |
| ATPase, class VI, type 11A | AW068936 | 6.18 | 7.56 |
| ATP-binding cassette, subfamily C (CFTR/MRP), | AI539710 | 6.92 | 8.23 |
| member 1 | | | |
| Baculoviral IAP repeat-containing 3 | AA805622 | 6.9 | 9.05 |
| BAI1-associated protein 2-like 1 | AA496034 | 6.55 | 8.19 |
| Basic helix-loop-helix domain containing, class | NM_003670 | 8.5 | 9.9 |
| B, 2 | | | |
| Basonuclin 2 | NM_017637 | 7.21 | 8.38 |
| B-cell CLL/lymphoma 6 (zinc finger protein 51) | NM_001706 | 6.88 | 8.75 |
| B-cell scaffold protein with ankyrin repeats 1 | BG200452 | 5.44 | 7.77 |
| BCL2/adenovirus E1B 19 kDa interacting protein 3 | U15174 | 6.61 | 7.95 |
| BCL2-related protein A1 | NM_004049 | 6.32 | 9.47 |
| β-Site APP-cleaving enzyme 2 | NM_012105 | 7.98 | 10.15 |
| B-factor, properdin | NM_001710 | 6.58 | 10.03 |
| BH3 interacting domain death agonist | BC005884 | 7.75 | 8.95 |
| BH3-only member B protein | NM_024949 | 5.09 | 6.75 |
| Biglycan | BC002416 | 3.27 | 7.17 |
| Biliverdin reductase A | NM_000712 | 7.79 | 9.28 |
| Brain abundant, membrane attached signal | NM_006317 | 8.98 | 10.66 |
| protein 1 | | | |
| Branched chain aminotransferase 1, cytosolic | AL390172 | 5.8 | 7.85 |
| Brother of CDO | W72626 | 5.88 | 6.89 |
| Bruton agammaglobulinemia tyrosine kinase | NM_000061 | 4.94 | 6.72 |
| BTB and CNC homology 1, basic leucine zipper | NM_021813 | 5.42 | 6.71 |
| transcription factor 2 | | | |
| BUB1 budding uninhibited by benzimidazoles 1 | AF043294 | 5.73 | 6.75 |
| homolog (yeast) | | | |
| Butyrophilin, subfamily 3, member A3 | NM_006994 | 6.87 | 8.02 |
| Cadherin 11, type 2, OB-cadherin (osteoblast) | NM_001797 | 5.97 | 7.2 |
| Cadherin 3, type 1, P-cadherin (placental) | NM_001793 | 4.89 | 8.75 |
| Cadherin 5, type 2, VE-cadherin (vascular | NM_001795 | 5.87 | 8.02 |
| epithelium) | | | |
| Calcitonin receptor-like | AI478743 | 4.67 | 6.87 |
| Calcium regulated heat stable protein 1, 24 kDa | NM_014316 | 8.09 | 9.31 |
| Caldesmon 1 | AL577531 | 9.18 | 10.34 |
| Calumenin | NM_001219 | 9.12 | 10.25 |
| cAMP responsive element modulator | NM_001881 | 6.44 | 7.61 |
| Cancer susceptibility candidate 5 | BF248364 | 6.46 | 7.87 |
| Carbohydrate (chondroitin) synthase 1 | NM_014918 | 7.44 | 8.66 |
| Carbohydrate (N-acetylglucosamine-6-O) | NM_004267 | 6.74 | 8.3 |
| sulfotransferase 2 | | | |
| Carboxypeptidase A3 (mast cell) | NM_001870 | 9.06 | 10.47 |
| Carcinoembryonic antigen-related cell adhesion | BC005008 | 11.91 | 13.08 |
| molecule 6 (nonspecific cross-reacting antigen) | | | |
| CARD only protein | NM_052889 | 8.75 | 10.23 |
| Caspase 1, apoptosis-related cysteine protease | AI719655 | 8.61 | 10.51 |
| (IL-1, β, convertase) | | | |
| Caspase 10, apoptosis-related cysteine protease | NM_001230 | 6.51 | 7.67 |
| Caspase 4, apoptosis-related cysteine protease | U25804 | 7.93 | 9.16 |
| Caspase 5, apoptosis-related cysteine protease | NM_004347 | 9.38 | 10.43 |
| Caspase recruitment domain family, member 15 | NM_022162 | 4.63 | 6.67 |
| Caspase recruitment domain family, member 6 | AF356193 | 4.69 | 6.88 |
| Cathepsin C | AI246687 | 8.67 | 9.69 |
| Cathepsin E | NM_001910 | 8.71 | 10.32 |
| Cathepsin H | NM_004390 | 8.87 | 10.5 |
| Cathepsin K (pycnodysostosis) | NM_000396 | 8.28 | 10.39 |
| Caveolin 1, caveolae protein, 22 kDa | AU147399 | 8.91 | 10.19 |
| Caveolin 2 | NM_001233 | 6.95 | 8.33 |
| CBL-interacting protein Sts-1 | AI418293 | 5.2 | 6.85 |
| CCAAT/enhancer binding protein (C/EBP), δ | NM_005195 | 9.75 | 10.87 |
| CD248 antigen, endosialin | NM_020404 | 6.29 | 7.73 |
| CD274 antigen | AI608902 | 5.65 | 8.1 |
| CD300A antigen | AF020314 | 6.39 | 7.66 |
| CD38 antigen (p45) | NM_001775 | 6.33 | 7.49 |
| CD3D antigen, δ polypeptide (TiT3 complex) | NM_000732 | 8.53 | 9.81 |
| CD40 antigen (TNF receptor superfamily member | NM_001250 | 5.38 | 7 |
| 5) | | | |
| CD44 antigen (homing function and Indian blood | M24915 | 7.9 | 9.6 |
| group system) | | | |
| CD47 antigen (Rh-related antigen, integrin- | Z25521 | 8.93 | 9.96 |
| associated signal transducer) | | | |
| CD48 antigen (B-cell membrane protein) | NM_001778 | 8.72 | 9.74 |
| CD52 antigen (CAMPATH-1 antigen) | NM_001803 | 8.88 | 9.95 |
| CD59 antigen p18-20 (antigen identified by | X16447 | 9.84 | 10.88 |
| monoclonal antibodies 16.3A5, EJ16, EJ30, EL32 | | | |
| and G344) | | | |
| CD68 antigen | NM_001251 | 5.33 | 6.75 |
| CD69 antigen (p60, early T-cell activation | L07555 | 7.37 | 8.56 |
| antigen) | | | |
| CD72 antigen | AF283777 | 5.42 | 6.88 |
| CD79A antigen (immunoglobulin-associated α) | NM_001783 | 8.2 | 9.53 |
| CD80 antigen (CD28 antigen ligand 1, B7-1 | B0042665 | 5.69 | 8.44 |
| antigen) | | | |
| CD86 antigen (CD28 antigen ligand 2, B7-2 | L25259 | 6.52 | 8.07 |
| antigen) | | | |
| CDC28 protein kinase regulatory subunit 2 | NM_001827 | 8.86 | 9.95 |
| Centromere protein E, 312 kDa | NM_001813 | 5.96 | 7.27 |
| Ceroid-lipofuscinosis, neuronal 8 (epilepsy, | AF123758 | 7.49 | 8.78 |
| progressive with mental retardation) | | | |
| Charcot-Leyden crystal protein /// Charcot- | NM_001828 | 6.26 | 8.01 |
| Leyden crystal protein | | | |
| Chemokine (C-C motif) ligand 11 | D49372 | 7.75 | 10.04 |
| Chemokine (C-C motif) ligand 18 (pulmonary and | AB000221 | 7.99 | 9.17 |
| activation regulated) | | | |
| Chemokine (C-C motif) ligand 19 | U88321 | 8.44 | 10.4 |
| Chemokine (C-C motif) ligand 2 | S69738 | 7.92 | 9.43 |
| Chemokine (C-C motif) ligand 20 | NM_004591 | 7.91 | 10.85 |
| Chemokine (C-C motif) ligand 21 | NM_002989 | 6.98 | 8.27 |
| Chemokine (C-C motif) ligand 3 /// chemokine (C- | NM_002983 | 6.34 | 8.43 |
| C motif) ligand 3-like 1 /// chemokine (C-C motif) | | | |
| ligand 3-like, centromeric | | | |
| Chemokine (C-C motif) ligand 4 | NM_002984 | 6.92 | 9.07 |
| Chemokine (C-C motif) receptor 1 | NM_001295 | 7.61 | 9.14 |
| Chemokine (C-C motif) receptor 7 | NM_001838 | 6.35 | 7.95 |
| Chemokine (C-C motif) receptor-like 1 | NM_016557 | 5.86 | 6.88 |
| Chemokine (C—X—C motif) ligand 1 (melanoma | NM_001511 | 6.94 | 11.68 |
| growth stimulating activity, α) | | | |
| Chemokine (C—X—C motif) ligand 10 | NM_001565 | 6.56 | 9.26 |
| Chemokine (C—X—C motif) ligand 11 | AF030514 | 4.08 | 8.34 |
| Chemokine (C—X—C motif) ligand 13 (B-cell | NM_006419 | 8.32 | 10.96 |
| chemoattractant) | | | |
| Chemokine (C—X—C motif) ligand 2 | M57731 | 5.16 | 9.02 |
| Chemokine (C—X—C motif) ligand 3 | NM_002090 | 6.47 | 10.1 |
| Chemokine (C—X—C motif) ligand 5 | AK026546 | 3.01 | 9.29 |
| Chemokine (C—X—C motif) ligand 6 (granulocyte | NM_002993 | 4.78 | 9.34 |
| chemotactic protein 2) | | | |
| Chemokine (C—X—C motif) ligand 9 | NM_002416 | 6.97 | 8.65 |
| Chemokine (C—X—C motif) receptor 4 | AJ224869 | 8.63 | 9.79 |
| Chemokine-like factor | NM_016951 | 7.97 | 9.38 |
| Chemokine-like factor super family 2 | AA778552 | 5.91 | 7.23 |
| Chemokine-like factor super family 3 | AL574900 | 6.7 | 7.89 |
| Chemokine-like factor super family 7 | AI708432 | 7.09 | 8.2 |
| Chitinase 3-like 1 (cartilage glycoprotein-39) | M80927 | 2.15 | 9.35 |
| Chitinase 3-like 2 | U58515 | 6.81 | 8.37 |
| Chloride intracellular channel 6 | AI638295 | 6.03 | 7.77 |
| Chondroitin sulfate GalNAcT-2 | NM_018590 | 7.28 | 8.62 |
| Chondroitin sulfate proteoglycan 2 (versican) | BF590263 | 8.02 | 9.25 |
| Chromobox homolog 4 (Pc class homolog, | AI570531 | 8.31 | 9.37 |
| Drosophila) | | | |
| Coactosin-like 1 (Dictyostelium) | NM_021615 | 8.19 | 9.44 |
| Coagulation factor II (thrombin) receptor | NM_001992 | 6.41 | 7.93 |
| Coagulation factor II (thrombin) receptor-like 2 | AI378647 | 5.67 | 8.33 |
| Coagulation factor III (thromboplastin, tissue | NM_001993 | 8.76 | 10.48 |
| factor) | | | |
| Coagulation factor V (proaccelerin, labile factor) | NM_000130 | 5.9 | 6.9 |
| Collagen triple helix repeat containing 1 | AA584310 | 4.46 | 8.22 |
| Collagen, type I, α 1 | K01228 | 10.4 | 12.09 |
| Collagen, type I, α 2 | NM_000089 | 7.96 | 10.75 |
| Collagen, type III, α 1 (Ehlers-Danlos syndrome | AI813758 | 10.72 | 11.99 |
| type IV, autosomal dominant) | | | |
| Collagen, type IV, α 1 | NM_001845 | 5.27 | 7.67 |
| Collagen, type IV, α 2 | AA909035 | 6.65 | 8.78 |
| Collagen, type V, α 1 | N30339 | 6.97 | 8.16 |
| Collagen, type V, α 2 | NM_000393 | 7.43 | 9.24 |
| Collagen, type VI, α 1 | AA292373 | 10.1 | 11.42 |
| Collagen, type VI, α 3 | NM_004369 | 8.51 | 10.88 |
| Collagen, type VII, α 1 (epidermolysis bullosa, | NM_000094 | 6.08 | 7.75 |
| dystrophic, dominant and recessive) | | | |
| Collagen, type XII, α 1 | AA788946 | 6.98 | 9.49 |
| Collagen, type XV, α 1 | NM_001855 | 7 | 8.65 |
| Collagen, type XVIII, α 1 | AF018081 | 7.29 | 8.75 |
| Colony-stimulating factor 3 receptor (granulocyte) | NM_000760 | 3.46 | 7.6 |
| Complement component (3d/Epstein-Barr virus) | NM_001877 | 7.49 | 9.65 |
| receptor 2 | | | |
| Complement component 1, q subcomponent, | NM_012072 | 7.32 | 9.36 |
| receptor 1 | | | |
| Complement component 1, r subcomponent | AL573058 | 8.42 | 9.84 |
| Complement component 1, r subcomponent-like | NM_016546 | 6.77 | 8.03 |
| Complement component 3 | NM_000064 | 7.86 | 10.36 |
| Complement component 4 binding protein, α | NM_000715 | 5.45 | 9.54 |
| Complement component 4 binding protein, β | NM_000716 | 4.55 | 8.61 |
| Complement component 5 receptor 1 (C5a | NM_001736 | 6.74 | 8.05 |
| ligand) | | | |
| Copine V | AW967768 | 6.62 | 7.76 |
| Core 1 synthase, glycoprotein-N- | NM_020156 | 7.58 | 8.66 |
| acetylgalactosamine 3-β-galactosyltransferase, 1 | | | |
| Coronin, actin binding protein, 1A | U34690 | 7.37 | 8.78 |
| Crystallin, ζ (quinone reductase) | NM_001889 | 7.84 | 9 |
| C-type lectin domain family 4, member A | NM_016184 | 6.86 | 8.27 |
| C-type lectin domain family 4, member E | BC000715 | 5.4 | 7.16 |
| C-type lectin domain family 7, member A | AF313468 | 6.88 | 8.33 |
| Cyclin B1 | BE407516 | 7.68 | 8.93 |
| Cyclin-dependent kinase inhibitor 3 (CDK2- | AF213033 | 7.48 | 8.81 |
| associated dual specificity phosphatase) | | | |
| Cystatin A (stefin A) | NM_005213 | 7.31 | 8.84 |
| Cysteine- and glycine-rich protein 2 | NM_001321 | 6.95 | 8.42 |
| Cysteine- and tyrosine-rich 1 | AI458003 | 5.73 | 7.36 |
| Cysteine-rich hydrophobic domain 1 | AA062610 | 6.45 | 7.61 |
| Cysteinyl leukotriene receptor 1 | NM_006639 | 7.12 | 8.17 |
| Cytochrome b-245, β polypeptide (chronic | NM_000397 | 7.92 | 9.09 |
| granulomatous disease) | | | |
| Cytochrome P450, family 2, subfamily C, | NM_000772 | 6.22 | 7.68 |
| polypeptide 18 /// cytochrome P450, family 2, | | | |
| subfamily C, polypeptide 18 | | | |
| Cytoplasmic FMR1 interacting protein 2 /// | NM_030778 | 6.72 | 7.77 |
| cytoplasmic FMR1 interacting protein 2 | | | |
| DDHD domain containing 1 | AA029818 | 5.8 | 6.83 |
| Decay accelerating factor for complement (CD55, | NM_000574 | 8.18 | 11.37 |
| Cromer blood group system) | | | |
| Decorin | AF138300 | 10.24 | 11.27 |
| Dedicator of cytokinesis 11 | AI742838 | 6.41 | 7.83 |
| Dedicator of cytokinesis 2 | D86964 | 6.69 | 7.8 |
| Dedicator of cytokinesis 4 | NM_014705 | 6.29 | 7.4 |
| Dedicator of cytokinesis 8 | AV760561 | 5.97 | 7.38 |
| Defensin, α 5, Paneth cell-specific | NM_021010 | 6.78 | 11.42 |
| Defensin, α 6, Paneth cell-specific | NM_001926 | 5.64 | 10.98 |
| Defensin, β 4 | NM_004942 | 5.12 | 10.13 |
| Degenerative spermatocyte homolog 1, lipid | BC000961 | 7.59 | 8.86 |
| desaturase (Drosophila) | | | |
| Dehydrogenase/reductase (SDR family) member 9 | NM_005771 | 10.85 | 11.88 |
| Deiodinase, iodothyronine, type II | NM_013989 | 6.18 | 7.19 |
| Deleted in malignant brain tumors 1 | NM_004406 | 7.99 | 11.68 |
| Dermatopontin | AI146848 | 5.42 | 6.79 |
| Desmuslin | AK026420 | 5.93 | 7.28 |
| Development and differentiation enhancing factor 1 | AW513835 | 7.93 | 9.12 |
| Dickkopf homolog 3 (Xenopus laevis) | NM_013253 | 5.43 | 6.79 |
| Discoidin domain receptor family, member 2 | W73819 | 9.17 | 10.24 |
| Discs, large homolog 7 (Drosophila) | NM_014750 | 6.94 | 8.04 |
| Disrupted in renal carcinoma 2 | AI147467 | 8.22 | 9.4 |
| DNA-damage-inducible transcript 4 | NM_019058 | 7.83 | 9.38 |
| DnaJ (Hsp40) homolog, subfamily C, member 10 | AA651899 | 6.57 | 7.75 |
| Docking protein 3 | BC004564 | 6 | 7.68 |
| DORA reverse strand protein 1 | AI536637 | 6.77 | 7.84 |
| Down-regulated by Ctnnb1, a | AV734839 | 7.15 | 8.35 |
| Down-regulated in ovarian cancer 1 | NM_014890 | 8.06 | 9.42 |
| Dual adaptor of phosphotyrosine and 3- | AA521016 | 6.59 | 7.74 |
| phosphoinositides | | | |
| Dual oxidase 2 | NM_014080 | 5.5 | 12.22 |
| Dual specificity phosphatase 10 | N36770 | 7.18 | 8.24 |
| Dual specificity phosphatase 7 | AI655015 | 7.09 | 8.16 |
| Duffy blood group | NM_002036 | 5.22 | 7.53 |
| E2F transcription factor 5, p130-binding | U15642 | 5.74 | 6.97 |
| Early B-cell factor | BG435302 | 3.64 | 7.95 |
| Early growth response 2 (Krox-20 homolog, | NM_000399 | 5.81 | 6.98 |
| Drosophila) | | | |
| Early growth response 3 | NM_004430 | 6.12 | 7.61 |
| Ecotropic viral integration site 2A | NM_014210 | 8.3 | 9.33 |
| Ecotropic viral integration site 2B | BC005926 | 8.66 | 9.74 |
| Ectonucleoside triphosphate diphosphohydrolase 1 | AV717590 | 7.75 | 8.75 |
| Ectonucleotide | L35594 | 8.3 | 9.64 |
| pyrophosphatase/phosphodiesterase 2 (autotaxin) | | | |
| EF hand domain family, member D2 | AW664179 | 8.79 | 9.96 |
| EGF, latrophilin and 7 transmembrane domain | NM_022159 | 6.62 | 8.82 |
| containing 1 | | | |
| EGF-like module containing, mucin-like, hormone | NM_013447 | 4.13 | 7.02 |
| receptor-like 2 | | | |
| EGF-like-domain, multiple 6 | NM_015507 | 3.96 | 6.85 |
| egl 9 homolog 3 (C elegans) | NM_022073 | 8.1 | 9.37 |
| ELK3, ETS-domain protein (SRF accessory | AW575374 | 7.2 | 9.25 |
| protein 2) | | | |
| Elongation factor, RNA polymerase II, 2 | NM_012081 | 5.38 | 7.37 |
| ELOVL family member 5, elongation of long | AL136939 | 8.31 | 10.01 |
| chain fatty acids (FEN1/Elo2, SUR4/Elo3-like, | | | |
| yeast) | | | |
| Endomucin | AI635774 | 5.86 | 6.88 |
| Endothelial cell growth factor 1 (platelet-derived) | NM_001953 | 6.54 | 8.32 |
| Endothelial differentiation, sphingolipid G-protein- | NM_001400 | 5.79 | 7.79 |
| coupled receptor, 1 | | | |
| Endothelial differentiation, sphingolipid G-protein- | AA534817 | 6.09 | 7.84 |
| coupled receptor, 3 | | | |
| Endothelin receptor type A | NM_001957 | 6.6 | 7.94 |
| Engulfment and cell motility 2 (ced-12 homolog, | BC000143 | 6.09 | 7.19 |
| C elegans) | | | |
| Ependymin-related protein 1 (zebrafish) | BC000686 | 6.61 | 7.64 |
| Epidermal retinal dehydrogenase 2 | AI440266 | 7.88 | 9.88 |
| Epiregulin | NM_001432 | 5.53 | 6.74 |
| Epithelial cell transforming sequence 2 oncogene | NM_018098 | 8.35 | 9.43 |
| Epithelial stromal interaction 1 (breast) | BE645480 | 6.74 | 7.81 |
| Epstein-Barr virus induced gene 2 (lymphocyte- | NM_004951 | 6.95 | 8.23 |
| specific G protein-coupled receptor) | | | |
| ERO1-like (S cerevisiae) | NM_014584 | 7.09 | 9.49 |
| Erythrocyte membrane protein band 4.1 | AW771958 | 6.3 | 7.35 |
| (elliptocytosis 1, RH-linked) | | | |
| Family with sequence similarity 20, member C | BE874872 | 7.6 | 8.61 |
| Family with sequence similarity 3, member B | BF106962 | 5.89 | 7.3 |
| Family with sequence similarity 49, member A | AA243659 | 5.49 | 6.67 |
| Family with sequence similarity 54, member A | AL138828 | 6.08 | 7.16 |
| Fas apoptotic inhibitory molecule | AI084226 | 7.32 | 9.05 |
| F-box protein 11 | AL117620 | 8.91 | 10.66 |
| F-box protein 6 | AF129536 | 6 | 7.47 |
| Fc fragment of IgE, high-affinity I, receptor for γ | NM_004106 | 8.47 | 9.49 |
| polypeptide | | | |
| Fc fragment of IgG, low-affinity IIa, receptor | NM_021642 | 6.65 | 7.9 |
| (CD32) | | | |
| Fc fragment of IgG, low-affinity IIIb, receptor | J04162 | 4.92 | 9.28 |
| (CD16b) | | | |
| Fc receptor-like 5 | AF343663 | 5.55 | 6.91 |
| FCH and double SH3 domains 2 | NM_014824 | 6.57 | 7.6 |
| Fibrillin 1 (Marfan syndrome) | NM_000138 | 8.43 | 9.75 |
| fibroblast growth factor 7 (keratinocyte growth | AF523265 | 6.4 | 7.9 |
| factor) ///keratinocyte growth factor-like protein 1 | | | |
| Fibronectin leucine-rich transmembrane protein 2 | NM_013231 | 6.61 | 8.17 |
| Fibronectin type III domain containing 3B | BF444916 | 7.05 | 8.17 |
| Filamin A interacting protein 1 | BC029425 | 5.68 | 6.78 |
| FK506-binding protein 11, 19 kDa | NM_016594 | 9.1 | 10.37 |
| Follistatin-like 1 | BC000055 | 9.65 | 10.98 |
| Forkhead box Q1 | AI676059 | 5.77 | 8.07 |
| Formyl peptide receptor 1 | NM_002029 | 5.71 | 9.41 |
| Formyl peptide receptor-like 1 | M88107 | 5.02 | 7.29 |
| Friend leukemia virus integration 1 | NM_002017 | 6.18 | 7.39 |
| Fucosyltransferase 2 (secretor status included) | BC001899 | 9.01 | 10.33 |
| Fucosyltransferase 6 (α [1,3] fucosyltransferase) | M98825 | 5.79 | 6.82 |
| Fucosyltransferase 8 (α [1,6] fucosyltransferase) | NM_004480 | 8.09 | 9.5 |
| FXYD domain containing ion transport regulator 5 | AF177940 | 7.14 | 8.32 |
| FYN binding protein (FYB-120/130) | AI633888 | 6.07 | 7.73 |
| FYN oncogene related to SRC, FGR, YES | M14333 | 7.72 | 8.91 |
| FYVE, RhoGEF and PH domain containing 5 | AW269340 | 5.79 | 7.11 |
| G patch domain containing 2 | NM_018040 | 5.72 | 6.83 |
| G protein-coupled receptor 109B | NM_006018 | 3.89 | 8.82 |
| G protein-coupled receptor 116 | BF941499 | 5.64 | 7.18 |
| G protein-coupled receptor 126 | AL033377 | 6.58 | 8.93 |
| G protein-coupled receptor 128 | NM_032787 | 7.18 | 9.43 |
| G protein-coupled receptor 18 | AF261135 | 6.14 | 7.81 |
| G protein-coupled receptor 65 | NM_003608 | 6.13 | 7.58 |
| G protein-coupled receptor kinase 5 | NM_005308 | 7.75 | 8.79 |
| G protein-coupled receptor, family C, group 5, | NM_003979 | 10.22 | 11.43 |
| member A | | | |
| Gap junction protein, α 1, 43 kDa (connexin 43) | NM_000165 | 9.42 | 10.64 |
| Gardner-Rasheed feline sarcoma viral (v-fgr) | NM_005248 | 6.77 | 8.04 |
| oncogene homolog | | | |
| GDP-mannose 4,6-dehydratase | NM_001500 | 8.77 | 9.79 |
| Gene differentially expressed in prostate | BC020934 | 6.93 | 7.96 |
| General transcription factor IIIA | AI241331 | 7.18 | 8.29 |
| Glia maturation factor, γ | NM_004877 | 7.36 | 8.84 |
| Glucocorticoid induced transcript 1 | AA058770 | 7.25 | 8.51 |
| Glutamate receptor interacting protein 2 | BG150485 | 5.87 | 8.1 |
| Glutamate receptor, ionotropic, N-methyl-d- | AL137422 | 5.72 | 6.74 |
| aspartate 3A | | | |
| Glutamate-cysteine ligase, modifier subunit | NM_002061 | 6.88 | 8.06 |
| Glutaminyl-peptide cyclotransferase (glutaminyl | NM_012413 | 6.65 | 8.35 |
| cyclase) | | | |
| Glutaredoxin (thioltransferase) | NM_002064 | 10.2 | 11.83 |
| Glutathione peroxidase 7 | AA406605 | 5.71 | 6.79 |
| Glycerol kinase | AJ252550 | 6.25 | 7.47 |
| Glycosyltransferase 8 domain containing 2 | W63754 | 6.42 | 7.45 |
| Glypican 6 | AU144140 | 6.35 | 7.37 |
| Golgi transport 1 homolog B (S cerevisiae) | NM_016072 | 7.38 | 8.72 |
| Granzyme K (serine protease, granzyme 3; | NM_002104 | 7.24 | 8.25 |
| tryptase II) | | | |
| Gremlin 1 homolog, cysteine knot superfamily | NM_013372 | 7.64 | 10.94 |
| (Xenopus laevis) | | | |
| GTPase, IMAP family member 1 | NM_130759 | 5.85 | 7.01 |
| GTPase, IMAP family member 2 | AI431931 | 7.31 | 8.48 |
| GTPase, IMAP family member 4 | NM_018326 | 7.82 | 9.13 |
| GTPase, IMAP family member 6 | NM_024711 | 5.66 | 6.95 |
| GTPase, IMAP family member 7 | AA858297 | 7.69 | 9.04 |
| Guanine nucleotide binding protein (G protein), α | NM_002068 | 4.81 | 7.51 |
| 15 (Gq class) | | | |
| Guanine nucleotide binding protein (G protein), γ | NM_004126 | 8.46 | 9.81 |
| 11 | | | |
| Guanylate binding protein 1, interferon-inducible, | NM_002053 | 8.18 | 10.37 |
| 67 kDa | | | |
| Guanylate binding protein 2, interferon-inducible | NM_004120 | 9.05 | 10.26 |
| Guanylate binding protein 5 | BG545653 | 5.98 | 8.13 |
| Guanylate cyclase 1, soluble, α 3 | AI719730 | 7.48 | 9.35 |
| HCV F-transactivated protein 1 | BF244081 | 9.22 | 10.27 |
| Hematopoietic cell-specific Lyn substrate 1 | NM_005335 | 8.57 | 9.86 |
| Hematopoietic protein 1 | BC001604 | 7.17 | 8.41 |
| Hematopoietically expressed homeobox | NM_001529 | 5.58 | 7.23 |
| Hemoglobin, α 2 | V00489 | 10.15 | 11.3 |
| Hemoglobin, β /// hemoglobin, β | M25079 | 10.67 | 11.85 |
| Hemoglobin, δ /// hemoglobin, δ | NM_000519 | 6.52 | 8.05 |
| Hemopoietic cell kinase | NM_002110 | 6.29 | 8.19 |
| Heparan sulfate (glucosamine) 3-O- | NM_005114 | 6.57 | 7.91 |
| sulfotransferase 1 | | | |
| Heparan sulfate (glucosamine) 3-O- | NM_006042 | 5.59 | 6.69 |
| sulfotransferase 3A1 | | | |
| Heparanase | NM_006665 | 7.36 | 8.68 |
| Hepatocyte growth factor (hepapoietin A; scatter | X16323 | 5.52 | 7.89 |
| factor) | | | |
| Hepatoma-derived growth factor, related protein 3 | AK001280 | 6.55 | 7.88 |
| Hermansky-Pudlak syndrome 1 | BF059516 | 5.69 | 6.78 |
| Hexokinase 2 | AI761561 | 10.61 | 11.85 |
| Hexokinase domain containing 1 | W81116 | 6.78 | 8.58 |
| Histone 1, H2bc | NM_003526 | 5.55 | 7.32 |
| Histone 2, H2aa | AI313324 | 8.56 | 9.77 |
| Histone deacetylase 9 | NM_014707 | 8.25 | 9.32 |
| Histone mRNA 3′ end-specific exonuclease | AL137679 | 6.38 | 7.39 |
| Homeo box B2 | NM_002145 | 5.98 | 7.09 |
| Homer homolog 1 (Drosophila) | BE550452 | 6.53 | 7.7 |
| Homogentisate 1,2-dioxygenase (homogentisate | NM_000187 | 8.04 | 9.04 |
| oxidase) | | | |
| HRAS-like suppressor 3 | BC001387 | 5.8 | 7.23 |
| HSPC054 protein | NM_014152 | 6.64 | 7.85 |
| HtrA serine peptidase 3 | AW518728 | 6.22 | 7.87 |
| Human immunodeficiency virus type I enhancer | AL023584 | 7.5 | 8.58 |
| binding protein 2 | | | |
| Huntington (Huntington disease) | NM_002111 | 7.44 | 8.45 |
| Hyaluronan and proteoglycan link protein 3 | BE348293 | 5.67 | 7.23 |
| Hyaluronan-mediated motility receptor (RHAMM) | NM_012485 | 6.89 | 8.42 |
| Hyaluronoglucosaminidase 1 | AF173154 | 5.13 | 7.26 |
| Hypoxia-inducible factor 1, α subunit (basic helix- | NM_001530 | 10.65 | 12.11 |
| loop-helix transcription factor) | | | |
| I factor (complement) | NM_000204 | 5.42 | 8.68 |
| IBR domain containing 3 | W27419 | 7.56 | 8.6 |
| IGF-II mRNA-binding protein 3 | AU160004 | 0 | 7.21 |
| IKK interacting protein | BF057681 | 5.36 | 6.75 |
| Immunoglobulin heavy constant μ | X17115 | 9.22 | 10.61 |
| Ig heavy locus /// Ig heavy constant γ 1 (G1m | M87789 | 11.68 | 13.61 |
| marker) /// γ 2 (G2m marker) /// γ 3 (G3m marker) | | | |
| /// Ig heavy constant μ | | | |
| Immunoglobulin λ variable 3-21 | AK025231 | 8.62 | 9.98 |
| Immunoglobulin superfamily containing leucine- | NM_005545 | 6.03 | 7.14 |
| rich repeat | | | |
| Immunoglobulin superfamily, member 4 | NM_014333 | 7.05 | 8.7 |
| Immunoglobulin superfamily, member 6 | NM_005849 | 7.24 | 8.54 |
| Indoleamine-pyrrole 2,3 dioxygenase | M34455 | 5.08 | 8.79 |
| Inhibin, β A (activin A, activin AB α polypeptide) | AI343467 | 3.78 | 7.69 |
| Inositol 1,4,5-triphosphate receptor, type 1 | NM_002222 | 7.6 | 9.13 |
| Insulin receptor substrate 1 | BG403162 | 5.85 | 7 |
| Insulin-like growth factor binding protein 2, 36 kDa | NM_000597 | 6.96 | 8.19 |
| Insulin-like growth factor binding protein 4 | NM_001552 | 9.1 | 10.15 |
| Insulin-like growth factor binding protein 5 | AW007532 | 7.86 | 10.56 |
| Insulin-like growth factor binding protein 7 | NM_001553 | 8.88 | 10.47 |
| Integrin, α 2 (CD49B, α 2 subunit of VLA-2 | N95414 | 7.53 | 9.54 |
| receptor) | | | |
| Integrin, α M (complement component receptor 3, | NM_000632 | 5.75 | 7.1 |
| α; also known as CD11b (p170), macrophage | | | |
| antigen α polypeptide) | | | |
| Integrin, α V (vitronectin receptor, α polypeptide, | AI093579 | 9.33 | 10.63 |
| antigen CD51) | | | |
| Integrin, β 2 (antigen CD18 (p95), lymphocyte | NM_000211 | 7.62 | 9.23 |
| function-associated antigen 1; macrophage | | | |
| antigen 1 [mac-1] β subunit) | | | |
| Integrin, β 6 | NM_000888 | 5.85 | 6.86 |
| Intelectin 1 (galactofuranose binding) | AB036706 | 11.91 | 13.16 |
| Intercellular adhesion molecule 1 (CD54), human | AI608725 | 6.24 | 7.27 |
| rhinovirus receptor | | | |
| Intercellular adhesion molecule 2 | AA126728 | 7.83 | 8.91 |
| Interferon (α, β, and ω) receptor 2 | BF526978 | 7.03 | 8.33 |
| Interferon induced transmembrane protein 1 (9-27) | NM_003641 | 10.42 | 11.62 |
| Interferon regulatory factor 1 | NM_002198 | 8.17 | 9.78 |
| Interferon stimulated gene 20 kDa | NM_002201 | 8.21 | 9.75 |
| Interferon, α-inducible protein (clone IFI-6-16) | NM_022873 | 7.12 | 8.19 |
| Interferon, γ-inducible protein 16 | NM_005531 | 9.42 | 10.7 |
| Interferon-induced protein with tetratricopeptide | AI075407 | 7.06 | 8.64 |
| repeats 3 | | | |
| IL-1 receptor accessory protein | NM_002182 | 6.38 | 7.43 |
| IL-1 receptor antagonist | U65590 | 4.97 | 9.55 |
| IL-1 receptor-like 1 | AI188516 | 5.78 | 6.79 |
| IL-1, β | NM_000576 | 7.26 | 9.83 |
| IL-13 receptor, α 2 | NM_000640 | 3.37 | 7.59 |
| IL-15 receptor, α | NM_002189 | 6.47 | 7.91 |
| IL-17 (cytotoxic T-lymphocyte-associated serine | Z58820 | 0 | 6.81 |
| esterase 8) | | | |
| IL-18 (interferon-γ-inducing factor) | NM_001562 | 7.29 | 8.66 |
| IL-7 receptor | NM_002185 | 8.93 | 10.3 |
| IL-8 | NM_000584 | 7.16 | 11.33 |
| IL-8 receptor, β | NM_001557 | 2.23 | 7.78 |
| Janus kinase 3 (a protein tyrosine kinase, | BF512748 | 6.46 | 8.28 |
| leukocyte) | | | |
| Junctional adhesion molecule 2 | NM_021219 | 6.05 | 7.62 |
| Juxtaposed with another zinc finger gene 1 | AL047908 | 6.63 | 7.64 |
| Kallikrein 10 | BC002710 | 3.37 | 9.34 |
| KARP-1-binding protein | NM_014812 | 8.2 | 9.45 |
| Katanin p60 subunit A-like 2 | AL512748 | 8.22 | 9.29 |
| KDEL (Lys-Asp-Glu-Leu) endoplasmic reticulum | NM_016657 | 7.42 | 8.86 |
| protein retention receptor 3 | | | |
| Kelch-like 5 (Drosophila) | AK002174 | 6.67 | 8.46 |
| Keratin 6A /// keratin 6C /// keratin 6E | J00269 | 2.22 | 6.8 |
| Keratin 6B | AI831452 | 4.92 | 7.48 |
| Kin of IRRE like (Drosophila) | AI049973 | 6.41 | 7.47 |
| Kinase insert domain receptor (a type III receptor | NM_002253 | 6.01 | 7.04 |
| tyrosine kinase) | | | |
| Kinesin family member 14 | NM_014875 | 6.36 | 7.56 |
| Kinetochore associated 2 | NM_006101 | 6.28 | 7.37 |
| Kynureninase (I-kynurenine hydrolase) | NM_003937 | 5.97 | 8.14 |
| Laminin, α 3 | NM_000227 | 8.36 | 9.63 |
| Laminin, γ 2 | NM_005562 | 8.14 | 10.29 |
| Latrophilin 2 | NM_012302 | 6.28 | 7.76 |
| LATS, large tumor suppressor, homolog 2 | AI535735 | 7.3 | 8.67 |
| (Drosophila) | | | |
| Leptin receptor | U50748 | 6.43 | 7.46 |
| Leucine-rich α-2-glycoprotein 1 | AA622495 | 7.01 | 8.22 |
| Leukocyte immunoglobulin-like receptor, | AI681260 | 5.55 | 7.28 |
| subfamily B (with TM and ITIM domains), member 1 | | | |
| Leukocyte-specific transcript 1 | AV713720 | 5.88 | 7.43 |
| Leukocyte-derived arginine aminopeptidase | NM_022350 | 6.86 | 7.89 |
| Likely ortholog of mouse limb-bud and heart gene | NM_030915 | 6.73 | 7.9 |
| Likely ortholog of mouse monocyte macrophage | NM_015957 | 8.93 | 10.76 |
| 19 | | | |
| Likely ortholog of mouse neighbor of Punc E11 | AB046848 | 4.57 | 6.78 |
| LIM domain binding 2 | NM_001290 | 6.04 | 7.6 |
| LIM domain only 2 (rhombotin-like 1) | NM_005574 | 7.4 | 8.85 |
| Lipase, endothelial | NM_006033 | 5.41 | 7.82 |
| Lipocalin 2 (oncogene 24p3) | NM_005564 | 8.52 | 13.05 |
| Lipoma HMGIC fusion partner | NM_005780 | 6.78 | 8.38 |
| Lipoprotein lipase | BF672975 | 5.78 | 6.85 |
| Low-density lipoprotein receptor-related protein | NM_017522 | 5.5 | 7.36 |
| 8, apolipoprotein e receptor | | | |
| Lumican | NM_002345 | 10.05 | 11.74 |
| Lung type-I cell membrane-associated | AU154455 | 5.34 | 6.96 |
| glycoprotein | | | |
| Lymphocyte antigen 64 homolog, radioprotective | NM_005582 | 6.54 | 7.62 |
| 105 kDa (mouse) | | | |
| Lymphocyte antigen 96 | NM_015364 | 7.84 | 9.68 |
| Lymphocyte cytosolic protein 1 (L-plastin) | J02923 | 8.14 | 9.56 |
| Lymphocyte cytosolic protein 2 (SH2 domain | NM_005565 | 6.95 | 8.03 |
| containing leukocyte protein of 76 kDa) | | | |
| Lymphocyte-specific protein tyrosine kinase | NM_005356 | 6.36 | 7.63 |
| Lymphoid enhancer-binding factor 1 | AF288571 | 6.26 | 8.23 |
| Lymphotoxin β (TNF superfamily, member 3) | NM_002341 | 7.72 | 9.1 |
| Lysophosphatidylglycerol acyltransferase 1 | NM_014873 | 8.47 | 9.55 |
| Lysosomal-associated membrane protein 3 | NM_014398 | 5.87 | 7.73 |
| Lysozyme (renal amyloidosis) | U25677 | 5.64 | 8.56 |
| Lysyl oxidase-like 2 | NM_002318 | 6.85 | 9.09 |
| Macrophage migration inhibitory factor | NM_002415 | 9.14 | 10.3 |
| (glycosylation-inhibiting factor) | | | |
| MADS box transcription enhancer factor 2, | AL530331 | 6.26 | 7.31 |
| polypeptide D (myocyte enhancer factor 2D) | | | |
| Major histocompatibility complex, class II, DQ α 1 | NM_002122 | 8.13 | 10.17 |
| Major histocompatibility complex, class II, DR α | M60334 | 10.59 | 12.01 |
| Major histocompatibility complex, class II, DR β 1 | AA807056 | 7.85 | 9.08 |
| Major histocompatibility complex, class II, DR β 4 | BC005312 | 7.69 | 9.32 |
| Malic enzyme 1, NADP(+)-dependent, cytosolic | NM_002395 | 8.14 | 10.18 |
| Matrix Gla protein | NM_000900 | 7.56 | 9.75 |
| Matrix metalloproteinase 1 (interstitial | NM_002421 | 7.13 | 11.18 |
| collagenase) | | | |
| Matrix metalloproteinase 10 (stromelysin 2) | NM_002425 | 4.89 | 8.79 |
| Matrix metalloproteinase 12 (macrophage | NM_002426 | 9.23 | 12.22 |
| elastase) | | | |
| Matrix metalloproteinase 2 (gelatinase A, 72 kDa | NM_004530 | 7.71 | 8.77 |
| gelatinase, 72 kDa type IV collagenase) | | | |
| Matrix metalloproteinase 3 (stromelysin 1, | NM_002422 | 5.62 | 10.85 |
| progelatinase) | | | |
| Matrix metalloproteinase 7 (matrilysin, uterine) | NM_002423 | 4.71 | 10.2 |
| Matrix metalloproteinase 9 (gelatinase B, 92 kDa | NM_004994 | 6.54 | 9.7 |
| gelatinase, 92 kDa type IV collagenase) | | | |
| MCP-1 treatment-induced protein | NM_025079 | 5.99 | 8.37 |
| Mediator of RNA polymerase II transcription, | BE645241 | 8.1 | 9.11 |
| subunit 28 homolog (yeast) | | | |
| Melanoma antigen family D, 4 | NM_030801 | 6.57 | 7.59 |
| Melanoma associated gene | BF342851 | 7.43 | 8.47 |
| Melanoma cell adhesion molecule | AF089868 | 6.92 | 7.96 |
| Melanoma inhibitory activity | NM_006533 | 5.5 | 7.67 |
| Membrane targeting (tandem) C2 domain | NM_152332 | 7.15 | 8.36 |
| containing 1 | | | |
| Membrane-associated ring finger (C3HC4) 1 | NM_017923 | 5.29 | 7.54 |
| Membrane-spanning 4-domains, subfamily A, | AW474852 | 8.74 | 10.7 |
| member 1 | | | |
| met proto-oncogene (hepatocyte growth factor | BG170541 | 8.1 | 9.3 |
| receptor) | | | |
| Methylenetetrahydrofolate dehydrogenase | NM_006636 | 9.32 | 10.38 |
| (NADP⁺ dependent) 2, methenyltetrahydrofolate | | | |
| cyclohydrolase | | | |
| Methylmalonic aciduria (cobalamin deficiency) | AW300959 | 6.01 | 7.22 |
| type A | | | |
| MHC class I polypeptide-related sequence B | NM_005931 | 6.06 | 7.2 |
| Midkine (neurite growth-promoting factor 2) | M69148 | 5.96 | 7.92 |
| Mitochondrial solute carrier protein | R92925 | 6.78 | 7.9 |
| Mitogen-activated protein kinase 1 | NM_138957 | 6.28 | 7.31 |
| Mitogen-activated protein kinase kinase 3 | AA780381 | 9.03 | 10.09 |
| Mitogen-activated protein kinase kinase kinase 8 | NM_005204 | 6.48 | 8.6 |
| Mitogen-activated protein kinase kinase kinase | NM_007181 | 6.32 | 7.59 |
| kinase 1 | | | |
| Mitogen-activated protein kinase kinase kinase | AL561281 | 6.28 | 7.41 |
| kinase 4 | | | |
| MLF1 interacting protein | NM_024629 | 7.45 | 8.66 |
| Moesin | NM_002444 | 9.1 | 10.4 |
| Monocyte to macrophage differentiation- | NM_012329 | 8.21 | 9.28 |
| associated | | | |
| Mucin 17 | AK026404 | 6.05 | 8.78 |
| Mucin 5, subtypes A and C, | AW192795 | 3.01 | 7.1 |
| tracheobronchial/gastric | | | |
| Multiple C2-domains with 2 transmembrane | NM_024717 | 6.12 | 7.96 |
| regions 1 | | | |
| Multiple coagulation factor deficiency 2 | BE880828 | 5.62 | 7.17 |
| Myeloid cell nuclear differentiation antigen | NM_002432 | 6.07 | 8.63 |
| Myeloid/lymphoid or mixed-lineage leukemia | AI023295 | 6.78 | 8.54 |
| (trithorax homolog, Drosophila); translocated to, | | | |
| 10 | | | |
| Myosin IB | BF432550 | 8.92 | 10.01 |
| Myosin IF | BF740152 | 7.27 | 8.36 |
| NAD(P)H dehydrogenase, quinone 1 | NM_000903 | 9.94 | 10.94 |
| NADPH cytochrome B5 oxidoreductase | NM_016230 | 8.08 | 9.12 |
| Neuregulin 1 | NM_004495 | 7.47 | 9.12 |
| Neurexin 3 | AI129949 | 6.17 | 7.56 |
| Neuromedin U | NM_006681 | 5.14 | 6.9 |
| Neuronal pentraxin II | U26662 | 5.32 | 8.39 |
| Neutrophil cytosolic factor 2 (65 kDa, chronic | BC001606 | 5.81 | 7.71 |
| granulomatous disease, autosomal 2) | | | |
| Neutrophil cytosolic factor 4, 40 kDa | NM_013416 | 6.15 | 7.37 |
| Nicotinamide N-methyltransferase | NM_006169 | 7.11 | 9.58 |
| Nidogen (enactin) | BF940043 | 7.98 | 9.02 |
| Nijmegen breakage syndrome 1 (nibrin) | AF049895 | 6.71 | 8.15 |
| Nitric oxide synthase 2A (inducible, hepatocytes) | L24553 | 4.9 | 10.27 |
| Notch homolog 3 (Drosophila) | NM_000435 | 6.61 | 8.07 |
| Nuclear factor of activated T cells, cytoplasmic, | U08015 | 5.52 | 6.97 |
| calcineurin-dependent 1 | | | |
| Nuclear factor of kappa light polypeptide gene | BE646573 | 9.76 | 11.26 |
| enhancer in B-cells inhibitor, ζ | | | |
| Nuclear localized factor 1 | BE218239 | 3.25 | 7.68 |
| Nuclear receptor coactivator 7 | AL035689 | 9.6 | 11.32 |
| Nuclear receptor subfamily 2, group F, member 1 | AI951185 | 7.54 | 8.72 |
| Nucleobindin 2 | NM_005013 | 9.02 | 10.22 |
| Nucleolar and coiled-body phosphoprotein 1 | NM_004741 | 7.07 | 8.45 |
| Nucleoredoxin | NM_017821 | 6.34 | 8.23 |
| Olfactomedin 1 | R38389 | 7.48 | 8.74 |
| Olfactomedin 4 | AL390736 | 8.94 | 12.91 |
| Oligodendrocyte transcription factor 1 | AL355743 | 5.42 | 6.67 |
| Oncostatin M receptor | AI133452 | 6.96 | 8.95 |
| Ovostatin 2 | AW594320 | 6.54 | 8.27 |
| Oxysterol binding protein-like 3 | AI202969 | 6.64 | 7.88 |
| Oxysterol binding protein-like 8 | AW978375 | 6.42 | 7.45 |
| Paired box gene 5 (B-cell lineage specific | BF510692 | 6.02 | 9.21 |
| activator) | | | |
| Paired related homeobox 1 | AA775472 | 3.63 | 6.85 |
| PALM2-AKAP2 protein | BG540494 | 8.62 | 9.76 |
| Palmdelphin | NM_017734 | 4.32 | 6.74 |
| Pannexin 1 | NM_015368 | 6.24 | 7.72 |
| Papilin, proteoglycan-like sulfated glycoprotein | AU145309 | 6.34 | 7.97 |
| PDZK1 interacting protein 1 | NM_005764 | 8 | 11.32 |
| Peptidylprolyl isomerase (cyclophilin)-like 1 | BC003048 | 6.87 | 8.02 |
| Periostin, osteoblast specific factor | AW137148 | 5.93 | 7.98 |
| Peroxiredoxin 4 | NM_006406 | 9.82 | 10.82 |
| PFTAIRE protein kinase 1 | NM_012395 | 6.77 | 7.77 |
| PH domain-containing protein | NM_025201 | 7.21 | 8.34 |
| PHD finger protein 17 | AW138134 | 7.92 | 9.14 |
| Phorbol-12-myristate-13-acetate-induced protein 1 | NM_021127 | 5.67 | 7.64 |
| Phosphatidylinositol 3,4,5-trisphosphate- | BF308645 | 5.51 | 7.12 |
| dependent RAC exchanger 1 | | | |
| Phosphatidylinositol transfer protein, cytoplasmic 1 | NM_012417 | 6.77 | 8.4 |
| Phosphodiesterase 4B, cAMP-specific | NM_002600 | 6.59 | 8.53 |
| (phosphodiesterase E4 dunce homolog, | | | |
| Drosophila) | | | |
| Phosphofructokinase, platelet | NM_002627 | 8.37 | 9.58 |
| Phosphoglucomutase 3 | BC001258 | 7.86 | 9.15 |
| Phosphoinositide-3-kinase adaptor protein 1 | AW575754 | 7.68 | 9.18 |
| Phosphoinositide-3-kinase, catalytic, δ | U86453 | 6.31 | 8.01 |
| polypeptide | | | |
| Phosphoinositide-3-kinase, regulatory subunit 3 | BE622627 | 6.2 | 7.86 |
| (p55, γ) | | | |
| Phosphoinositide-3-kinase, regulatory subunit 5, | BG236366 | 5.66 | 7.08 |
| p101 | | | |
| Phospholamban | NM_002667 | 5.47 | 7.21 |
| Phospholipase A2, group IIA (platelets, synovial | NM_000300 | 10.94 | 12 |
| fluid) | | | |
| Phospholipase A2, group VII (platelet-activating | NM_005084 | 7.38 | 8.84 |
| factor acetylhydrolase, plasma) | | | |
| Phosphoprotein enriched in astrocytes 15 | NM_003768 | 8.07 | 9.29 |
| Phosphoserine aminotransferase 1 | BC004863 | 6.75 | 7.92 |
| pim-1 oncogene | M24779 | 7.33 | 8.33 |
| pim-2 oncogene | NM_006875 | 7.25 | 8.79 |
| pim-3 oncogene | BE778706 | 8.18 | 9.25 |
| Pirin (iron-binding nuclear protein) | NM_003662 | 5.33 | 7.08 |
| Plasminogen activator, urokinase | NM_002658 | 6.27 | 9.08 |
| Plasminogen activator, urokinase receptor | X74039 | 6.91 | 8.38 |
| Plastin 3 (T isoform) | NM_005032 | 8.48 | 9.99 |
| Platelet/endothelial cell adhesion molecule | AA702701 | 7.04 | 8.85 |
| (CD31 antigen) | | | |
| Platelet-derived growth factor receptor, β | NM_002609 | 7.92 | 9.51 |
| polypeptide | | | |
| Pleckstrin | NM_002664 | 7.28 | 9.68 |
| Pleckstrin homology domain containing, family C | AI928241 | 7.16 | 8.33 |
| (with FERM domain) member 1 | | | |
| Pleckstrin homology, Sec7 and coiled-coil | NM_013385 | 6.82 | 8.04 |
| domains 4 | | | |
| Pleckstrin homology, Sec7 and coiled-coil | L06633 | 7.51 | 8.96 |
| domains, binding protein | | | |
| Pleckstrin homology-like domain, family A, | AI795908 | 6.24 | 7.75 |
| member 1 | | | |
| Pleckstrin homology-like domain, family B, | AK025444 | 7.83 | 8.92 |
| member 2 | | | |
| Polo-like kinase 2 (Drosophila) | NM_006622 | 7.29 | 8.49 |
| Poly (ADP-ribose) polymerase family, member 14 | AW297731 | 6.09 | 7.83 |
| Poly (ADP-ribose) polymerase family, member 9 | AF307338 | 7.57 | 9.14 |
| POU domain, class 2, associating factor 1 | NM_006235 | 8.22 | 9.83 |
| POU domain, class 2, transcription factor 2 | AA805754 | 5.57 | 6.76 |
| Pre-B-cell colony enhancing factor 1 | NM_005746 | 9.84 | 11.61 |
| Pregnancy-specific β-1-glycoprotein 6 | NM_002782 | 10.32 | 11.37 |
| Pregnancy-associated plasma protein A, | BF107618 | 6.4 | 8.34 |
| pappalysin 1 | | | |
| Pro-apoptotic caspase adaptor protein | NM_016459 | 7.94 | 9.11 |
| Prokineticin 2 | AF182069 | 4.21 | 9.43 |
| Pro-platelet basic protein (chemokine [C—X—C | R64130 | 4.42 | 6.66 |
| motif] ligand 7) | | | |
| Proprotein convertase subtilisin/kexin type 1 | NM_000439 | 5.69 | 7.46 |
| Prospero-related homeobox 1 | AK025453 | 7.34 | 8.38 |
| Prostaglandin D2 synthase 21 kDa (brain) | NM_000954 | 7.67 | 9.5 |
| Prostaglandin-endoperoxide synthase 2 | NM_000963 | 7.13 | 8.4 |
| (prostaglandin G/H synthase and cyclo- | | | |
| oxygenase) | | | |
| Protease inhibitor 15 | AI088609 | 6.11 | 8.05 |
| Protease inhibitor 3, skin-derived (SKALP) | NM_002638 | 8.82 | 12.74 |
| Protease, serine, 11 (IGF binding) | NM_002775 | 7.31 | 8.45 |
| Proteasome (prosome, macropain) activator | BC002684 | 6.86 | 7.91 |
| subunit 3 (PA28 γ; Ki) | | | |
| Proteasome (prosome, macropain) subunit, β | NM_002800 | 9.2 | 10.95 |
| type, 9 (large multifunctional protease 2) | | | |
| Protein C receptor, endothelial (EPCR) | NM_006404 | 7 | 8.38 |
| Protein kinase C, δ binding protein | AI088622 | 7.21 | 8.22 |
| Protein kinase C, η | NM_024064 | 6.48 | 7.53 |
| Protein kinase, cAMP-dependent, regulatory, | NM_002736 | 7.87 | 9.03 |
| type II, β | | | |
| Protein phosphatase 1, regulatory (inhibitor) | AB020630 | 5.89 | 7.12 |
| subunit 16B | | | |
| Protein phosphatase 2C, magnesium-dependent, | BG542521 | 8.39 | 9.49 |
| catalytic subunit | | | |
| Protein tyrosine phosphatase type IVA, member 3 | BC003105 | 7.06 | 8.5 |
| Protein tyrosine phosphatase, nonreceptor type | AB023430 | 6.53 | 7.57 |
| substrate 1 | | | |
| Protein tyrosine phosphatase, receptor type, C | NM_002838 | 7.27 | 8.97 |
| Protein tyrosine phosphatase, receptor type, G | NM_002841 | 5.55 | 6.67 |
| Protein tyrosine phosphatase, receptor type, M | BC029442 | 5.98 | 7.23 |
| Proteoglycan 1, secretory granule | NM_002727 | 11.22 | 12.5 |
| PTPL1-associated RhoGAP 1 | NM_004815 | 6.46 | 7.57 |
| Purinergic receptor P2X, ligand-gated ion | U49396 | 5.13 | 6.91 |
| channel, 5 | | | |
| Purinergic receptor P2Y, G-protein coupled, 10 | NM_014499 | 6.27 | 7.41 |
| Purinergic receptor P2Y, G-protein coupled, 13 | NM_023914 | 6.08 | 7.61 |
| Purinergic receptor P2Y, G-protein coupled, 14 | NM_014879 | 7.39 | 8.52 |
| Putative insulin-like growth factor II-associated | X07868 | 6.12 | 7.68 |
| protein | | | |
| Putative lymphocyte G0/G1 switch gene | NM_015714 | 7.09 | 10.39 |
| Pyruvate dehydrogenase kinase, isoenzyme 3 | NM_005391 | 3.65 | 6.82 |
| Pyruvate kinase, muscle | NM_002654 | 9.6 | 10.92 |
| Quaking homolog, KH domain RNA binding | AL031781 | 7.09 | 8.11 |
| (mouse) | | | |
| Quiescin Q6 | NM_002826 | 8.63 | 9.7 |
| RAB GTPase activating protein 1-like | BG107203 | 7.86 | 8.94 |
| RAB31, member RAS oncogene family | NM_006868 | 7.52 | 9.59 |
| RAB34, member RAS oncogene family | AF322067 | 7.15 | 8.4 |
| RAB7, member RAS oncogene family-like 1 | BG338251 | 6.47 | 8.01 |
| RAB8B, member RAS oncogene family | AI807023 | 7.78 | 9.23 |
| Rac/Cdc42 guanine nucleotide exchange factor | D25304 | 7.09 | 8.15 |
| (GEF) 6 | | | |
| Raft-linking protein | D42043 | 8.24 | 9.62 |
| Ras association (RaIGDS/AF-6) domain family 2 | NM_014737 | 6.66 | 7.79 |
| Ras association (RaIGDS/AF-6) domain family 5 | BC004270 | 7.64 | 9 |
| RAS guanyl releasing protein 1 (calcium and | NM_005739 | 7.28 | 8.88 |
| DAG-regulated) | | | |
| ras homolog gene family, member H | NM_004310 | 6.96 | 8.31 |
| ras homolog gene family, member Q | NM_012249 | 7.09 | 8.19 |
| RasGEF domain family, member 1B | BF110534 | 7.81 | 9.01 |
| Ras-induced senescence 1 | BF062629 | 5.89 | 8.57 |
| ras-related C3 botulinum toxin substrate 2 (ρ | BE138888 | 9.07 | 10.44 |
| family, small GTP binding protein Rac2) | | | |
| RecQ protein-like (DNA helicase Q1-like) | AI962943 | 7.72 | 8.85 |
| Regenerating islet-derived 1 α (pancreatic stone | AF172331 | 0 | 12.48 |
| protein, pancreatic thread protein) | | | |
| Regenerating islet-derived 1 β (pancreatic stone | NM_006507 | 4.33 | 11.63 |
| protein, pancreatic thread protein) | | | |
| Regenerating islet-derived 3 α | NM_002580 | 0.86 | 10.32 |
| Regenerating islet-derived family, member 4 | AY007243 | 8.73 | 12.56 |
| Regenerating islet-derived-like, pancreatic stone | NM_006508 | 0 | 11.19 |
| protein-like, pancreatic thread protein-like (rat) | | | |
| Regucalcin (senescence marker protein-30) | D31815 | 3.86 | 6.68 |
| Regulated in glioma | NM_006394 | 7.03 | 8.33 |
| Regulator of G-protein signaling 13 | AF030107 | 6.69 | 7.82 |
| Regulator of G-protein signaling 18 | AF076642 | 4.68 | 6.76 |
| Regulator of G-protein signaling 19 | NM_005873 | 7.03 | 8.23 |
| Regulator of G-protein signaling 5 | NM_025226 | 8.88 | 10.81 |
| Regulatory factor X, 5 (influences HLA class II | AW027312 | 7.1 | 8.11 |
| expression) | | | |
| Response gene to complement 32 | NM_014059 | 6.68 | 8.2 |
| Restin (Reed-Steinberg cell-expressed | BF673049 | 5.75 | 6.95 |
| intermediate filament-associated protein) | | | |
| Retinoic acid induced 2 | NM_021785 | 6.44 | 7.44 |
| Retinol-binding protein 1, cellular | NM_002899 | 6.9 | 7.92 |
| Retinol dehydrogenase 10 (all-trans) | AW150720 | 8.08 | 9.12 |
| Rho family GTPase 1 | U69563 | 5.31 | 7.86 |
| Rho GDP dissociation inhibitor (GDI) β | NM_001175 | 9.98 | 11.12 |
| Rho GTPase activating protein 15 | NM_018460 | 7.1 | 8.39 |
| Rho GTPase activating protein 28 | AI935647 | 5.75 | 6.91 |
| Rho GTPase activating protein 9 | BC006107 | 7.31 | 8.52 |
| Rhomboid, veinlet-like 6 (Drosophila) | NM_024599 | 6.27 | 7.49 |
| Ribonuclease, RNase A family, k6 | NM_005615 | 8.13 | 9.22 |
| Ribonucleotide reductase M2 polypeptide | BE966236 | 7.53 | 8.73 |
| Ribosomal protein L39-like | L05096 | 6.4 | 7.88 |
| Ribosomal protein S6 kinase, 90 kDa, | AI992251 | 6.88 | 7.98 |
| polypeptide 2 | | | |
| Ring finger protein 183 | BE796148 | 6.14 | 7.26 |
| RNA-binding motif, single-stranded interacting | NM_016837 | 7.39 | 8.46 |
| protein 1 | | | |
| RNA-binding protein with multiple splicing | NM_006867 | 5.33 | 7.53 |
| Roundabout, axon guidance receptor, homolog 1 | BF059159 | 7.46 | 9.4 |
| (Drosophila) | | | |
| Runt-related transcription factor 1 (acute myeloid | AK026743 | 6.1 | 7.65 |
| leukemia 1; aml1 oncogene) | | | |
| Runt-related transcription factor 1; translocated | NM_004349 | 7.53 | 8.56 |
| to, 1 (cyclin D-related) | | | |
| Runt-related transcription factor 3 | NM_004350 | 6.75 | 8.33 |
| S100 calcium binding protein A11 (calgizzarin) | NM_005620 | 9.91 | 11.66 |
| S100 calcium binding protein A12 (calgranulin C) | NM_005621 | 2.99 | 7.66 |
| S100 calcium binding protein A2 | NM_005978 | 5.7 | 7.25 |
| S100 calcium binding protein A8 (calgranulin A) | NM_002964 | 5.22 | 11.62 |
| S100 calcium binding protein A9 (calgranulin B) | NM_002965 | 4.06 | 10.14 |
| S100 calcium binding protein P | NM_005980 | 10.73 | 12.6 |
| SAM domain and HD domain 1 | AV715309 | 7.29 | 8.46 |
| SAM domain, SH3 domain and nuclear | NM_022136 | 7.26 | 9.2 |
| localization signals, 1 | | | |
| SAR1a gene homolog 1 (S cerevisiae) | NM_020150 | 7.81 | 8.97 |
| Sarcoglycan, δ (35 kDa dystrophin-associated | AA479286 | 6.63 | 7.75 |
| glycoprotein) | | | |
| SEC14 and spectrin domains 1 | AW409611 | 8.18 | 9.37 |
| SEC14-like 1 (S cerevisiae) | NM_003003 | 7.93 | 9 |
| SEC24 related gene family, member D (S | NM_014822 | 8.9 | 10.33 |
| cerevisiae) | | | |
| Secreted frizzled-related protein 2 | AF311912 | 3.05 | 8.15 |
| Secreted phosphoprotein 1 (osteopontin, bone | M83248 | 6.07 | 9.19 |
| sialoprotein I, early T-lymphocyte activation 1) | | | |
| Secreted protein, acidic, cysteine-rich | NM_003118 | 9.63 | 11.15 |
| (osteonectin) | | | |
| Selectin E (endothelial adhesion molecule 1) | NM_000450 | 5.01 | 6.77 |
| Selectin L (lymphocyte adhesion molecule 1) | NM_000655 | 5.88 | 8.73 |
| Selectin P (granule membrane protein 140 kDa, | NM_003005 | 6.44 | 7.57 |
| antigen CD62) | | | |
| Septin 6 | D50918 | 5.32 | 6.74 |
| Serine (or cysteine) proteinase inhibitor, clade A | NM_000295 | 10.44 | 11.58 |
| (α-1 antiproteinase, antitrypsin), member 1 | | | |
| Serine (or cysteine) proteinase inhibitor, clade A | NM_001085 | 5.51 | 9.81 |
| (α-1 antiproteinase, antitrypsin), member 3 | | | |
| Serine (or cysteine) proteinase inhibitor, clade B | NM_030666 | 10 | 11.27 |
| (ovalbumin), member 1 | | | |
| Serine (or cysteine) proteinase inhibitor, clade B | U19556 | 6.94 | 8.1 |
| (ovalbumin), member 3 | | | |
| Serine (or cysteine) proteinase inhibitor, clade B | U19557 | 5.57 | 7.13 |
| (ovalbumin), member 4 | | | |
| Serine (or cysteine) proteinase inhibitor, clade B | NM_002639 | 7.72 | 10.83 |
| (ovalbumin), member 5 | | | |
| Serine (or cysteine) proteinase inhibitor, clade B | AW238005 | 7.62 | 9.29 |
| (ovalbumin), member 6 | | | |
| Serine (or cysteine) proteinase inhibitor, clade B | NM_003784 | 0 | 9.24 |
| (ovalbumin), member 7 | | | |
| Serine (or cysteine) proteinase inhibitor, clade B | NM_002640 | 6.54 | 7.88 |
| (ovalbumin), member 8 | | | |
| Serine (or cysteine) proteinase inhibitor, clade B | AI986192 | 7.75 | 9.42 |
| (ovalbumin), member 9 | | | |
| Serine (or cysteine) proteinase inhibitor, clade E | AL541302 | 8.21 | 9.61 |
| (nexin, plasminogen activator inhibitor type 1), | | | |
| member 2 | | | |
| Serine (or cysteine) proteinase inhibitor, clade I | NM_005025 | 4.61 | 7.06 |
| (neuroserpin), member 1 | | | |
| Serine protease inhibitor, Kazal type 4 | NM_014471 | 10.49 | 12.71 |
| Serum amyloid A2 | NM_030754 | 2.13 | 8.27 |
| SH2 domain containing 3C | AW665063 | 6.02 | 7.14 |
| SH2 domain protein 1A, Duncan's disease | AF072930 | 5.56 | 7.45 |
| (lymphoproliferative syndrome) | | | |
| SH3-domain binding protein 5 (BTK-associated) | AL562152 | 7.05 | 8.25 |
| SHC SH2-domain binding protein 1 | NM_024745 | 5.28 | 6.75 |
| Sialic acid binding Ig-like lectin 10 | AF301007 | 7.13 | 8.34 |
| Signal peptidase complex subunit 3 homolog (S | NM_021928 | 7.56 | 8.74 |
| cerevisiae) | | | |
| Signal sequence receptor, α (translocon- | AI016620 | 8.62 | 9.68 |
| associated protein α) | | | |
| Signal transducer and activator of transcription 1, | BC002704 | 7.16 | 9.04 |
| 91 kDa | | | |
| SLAM family member 7 | AL121985 | 6.84 | 8.19 |
| SLAM family member 8 | NM_020125 | 6.82 | 7.87 |
| Slingshot homolog 2 (Drosophila) | AA975530 | 8.16 | 9.29 |
| SMAD, mothers against DPP homolog 5 | NM_005903 | 4.34 | 7.22 |
| (Drosophila) | | | |
| Small proline-rich protein 1B (cornifin) | NM_003125 | 5.68 | 6.84 |
| SMC2 structural maintenance of chromosomes | NM_006444 | 6.46 | 7.48 |
| 2-like 1 (yeast) | | | |
| SNARE protein Ykt6 | NM_006555 | 6.07 | 7.11 |
| Solute carrier family 1 (glutamate/neutral amino | BF510711 | 6.85 | 7.97 |
| acid transporter), member 4 | | | |
| Solute carrier family 15 (H+/peptide transporter), | AA836116 | 7.16 | 8.63 |
| member 2 | | | |
| Solute carrier family 15, member 4 | AI636759 | 6.45 | 7.51 |
| Solute carrier family 16 (monocarboxylic acid | R15072 | 5.88 | 8.54 |
| transporters), member 14 | | | |
| Solute carrier family 16 (monocarboxylic acid | NM_004207 | 8.52 | 9.69 |
| transporters), member 3 | | | |
| Solute carrier family 16 (monocarboxylic acid | NM_004696 | 4.21 | 6.71 |
| transporters), member 4 | | | |
| Solute carrier family 2 (facilitated glucose | NM_006931 | 6.15 | 7.85 |
| transporter), member 3 | | | |
| Solute carrier family 28 (sodium-coupled | NM_004212 | 6.37 | 8.06 |
| nucleoside transporter), member 2 | | | |
| Solute carrier family 40 (iron-regulated | AA588092 | 7.28 | 8.52 |
| transporter), member 1 | | | |
| Solute carrier family 5 (sodium/glucose | NM_000343 | 6.77 | 8.07 |
| cotransporter), member 1 | | | |
| Solute carrier family 6 (amino acid transporter), | NM_007231 | 3.03 | 10.72 |
| member 14 | | | |
| Solute carrier family 6 (neurotransmitter | U17986 | 8.02 | 9.2 |
| transporter, creatine), member 8 | | | |
| Solute carrier family 6 (neurotransmitter | U41163 | 7.73 | 8.79 |
| transporter, creatine), member 8 /// similar to | | | |
| sodium- and chloride-dependent creatine | | | |
| transporter | | | |
| Solute carrier family 6 (proline IMINO | NM_020208 | 6.31 | 7.53 |
| transporter), member 20 | | | |
| Solute carrier family 7 (cationic amino acid | NM_014270 | 5.06 | 8.16 |
| transporter, y+ system), member 9 | | | |
| Solute carrier family 7, (cationic amino acid | NM_014331 | 5.59 | 6.8 |
| transporter, y+ system) member 11 | | | |
| Solute carrier family 8 (sodium/calcium | BF223010 | 6.73 | 7.8 |
| exchanger), member 1 | | | |
| Solute carrier organic anion transporter family, | NM_016354 | 6.5 | 8.44 |
| member 4A1 | | | |
| Sorbitol dehydrogenase | NM_003104 | 6.06 | 7.5 |
| Sorting nexin 10 | NM_013322 | 6.95 | 8.73 |
| SP110 nuclear body protein | NM_004509 | 7.55 | 8.9 |
| SPARC related modular calcium binding 2 | AB014737 | 5.96 | 7.1 |
| sparc/osteonectin, cwcv and kazal-like domains | AF231124 | 6.08 | 7.11 |
| proteoglycan (testican) | | | |
| sparc/osteonectin, cwcv and kazal-like domains | NM_014767 | 7.45 | 8.64 |
| proteoglycan (testican) 2 | | | |
| SPARC-like 1 (mast9, hevin) | NM_004684 | 10.07 | 11.09 |
| Spleen focus forming virus (SFFV) proviral | NM_003120 | 5.81 | 7.59 |
| integration oncogene spi1 | | | |
| Spondin 2, extracellular matrix protein | NM_012445 | 6.99 | 8.93 |
| Squalene epoxidase | AF098865 | 7.69 | 8.73 |
| Squamous cell carcinoma antigen recognized by | NM_013352 | 7.05 | 8.33 |
| T cells 2 | | | |
| Src-like-adaptor /// Src-like-adaptor | NM_006748 | 7.01 | 8.61 |
| ST8 α-N-acetyl-neuraminide α-2,8- | AA352113 | 6 | 7.33 |
| sialyltransferase 4 | | | |
| ST8 α-l-acetyl-neuraminide α-2,8- | AW015140 | 5.56 | 6.73 |
| sialyltransferase 6 | | | |
| Stannin | AF070673 | 6.32 | 8.04 |
| Stanniocalcin 1 | AI300520 | 5.86 | 7.54 |
| START domain containing 4, sterol regulated | AA628398 | 7.7 | 8.72 |
| Stearoyl-CoA desaturase (δ-9-desaturase) | AB032261 | 9.04 | 10.3 |
| Sterile α motif and leucine zipper containing | NM_016653 | 5.59 | 6.83 |
| kinase AZK | | | |
| Sterile α motif domain containing 9 | NM_017654 | 8.25 | 9.35 |
| Steroid 5 α-reductase 2-like | BC002480 | 8.28 | 10.37 |
| Steroid-sensitive gene 1 | AW303375 | 6.3 | 7.56 |
| Steroid sulfatase (microsomal), arylsulfatase C, | AI122754 | 6.92 | 8.96 |
| isozyme S | | | |
| Stomatin | AI537887 | 8.02 | 9.93 |
| Stress 70 protein chaperone, microsome- | NM_006948 | 7.39 | 8.72 |
| associated, 60 kDa | | | |
| Sulfatase 1 | BE500977 | 7.49 | 8.76 |
| Sulfotransferase family, cytosolic, 1C, member 1 | AF186255 | 5.55 | 7.43 |
| Superoxide dismutase 2, mitochondrial | W46388 | 7.89 | 9.84 |
| Supervillin | BF000697 | 6.29 | 7.42 |
| Suppressor of cytokine signaling 1 | AB005043 | 5.8 | 8.16 |
| Suppressor of cytokine signaling 3 | AI244908 | 6.71 | 9.83 |
| SWAP-70 protein | AI139569 | 7.85 | 9.44 |
| SWI/SNF related, matrix associated, actin | NM_003069 | 5.7 | 6.91 |
| dependent regulator of chromatin, subfamily a, | | | |
| member 1 | | | |
| Synaptopodin 2 | AA541622 | 7.95 | 9.04 |
| Synaptotagmin binding, cytoplasmic RNA | AF037448 | 7.34 | 8.46 |
| interacting protein | | | |
| Syndecan 2 (heparan sulfate proteoglycan 1, cell | AI380298 | 6.5 | 7.79 |
| surface-associated, fibroglycan) | | | |
| T-cell receptor α constant /// T-cell receptor α | M12959 | 8.78 | 9.87 |
| constant | | | |
| T-cell activation GTPase activating protein | BF591040 | 6.88 | 8.86 |
| TEK tyrosine kinase, endothelial (venous | NM_000459 | 6.46 | 7.92 |
| malformations, multiple cutaneous, and mucosal) | | | |
| Tenascin C (hexabrachion) | NM_002160 | 6.17 | 9.7 |
| Tetracycline transporter-like protein | L34409 | 6.43 | 7.69 |
| Thiamin pyrophosphokinase 1 | NM_022445 | 7.52 | 9.53 |
| Thrombomodulin | NM_000361 | 5 | 6.96 |
| Thrombospondin 2 | NM_003247 | 4.22 | 7.41 |
| Thromboxane A synthase 1 (platelet, cytochrome | NM_030984 | 6.88 | 8.33 |
| P450, family 5, subfamily A) /// thromboxane A | | | |
| synthase 1 (platelet, cytochrome P450, family 5, | | | |
| subfamily A) | | | |
| Thy-1 cell surface antigen | AA218868 | 6.77 | 9.03 |
| Tissue factor pathway inhibitor (lipoprotein- | BF511231 | 7.33 | 8.73 |
| associated coagulation inhibitor) | | | |
| Tissue factor pathway inhibitor 2 | L27624 | 4.71 | 6.75 |
| Tissue inhibitor of metalloproteinase 1 (erythroid | NM_003254 | 9.61 | 11.76 |
| potentiating activity, collagenase inhibitor) | | | |
| Tissue inhibitor of metalloproteinase 3 (Sorsby | NM_000362 | 8.3 | 9.38 |
| fundus dystrophy, pseudoinflammatory) | | | |
| Tissue-specific transplantation antigen P35B | NM_003313 | 8.25 | 9.34 |
| TNFAIP3-interacting protein 3 | NM_024873 | 5.79 | 9.42 |
| Toll-like receptor 2 | NM_003264 | 5.9 | 7.38 |
| Toll-like receptor 4 /// Toll-like receptor 4 | U93091 | 6.14 | 7.26 |
| Toll-like receptor 8 | AW872374 | 6.34 | 7.99 |
| TPA-regulated locus | NM_018475 | 8.4 | 9.86 |
| TRAF3 interacting protein 2 | AW296296 | 5.48 | 6.93 |
| TRAF3-interacting Jun N-terminal kinase (JNK)- | NM_025228 | 5.75 | 7.41 |
| activating modulator | | | |
| TRAF-interacting protein with a forkhead- | AA195074 | 6.97 | 8.39 |
| associated domain | | | |
| Transcobalamin I (vitamin B₁₂ binding protein, R | NM_001062 | 5.3 | 9.91 |
| binder family) | | | |
| Transcription elongation factor A (SII)-like 7 | BF591534 | 5.98 | 7.04 |
| Transcription factor 3 (E2A immunoglobulin | AI655986 | 5.66 | 7.1 |
| enhancer binding factors E12/E47) | | | |
| Transcription factor 4 | NM_003199 | 9.23 | 10.25 |
| Transcription factor 8 (represses IL-2 expression) | AI373166 | 5.98 | 7.21 |
| Transcription factor CP2-like 2 | BE566136 | 4.89 | 6.66 |
| Transforming growth factor, β 1 (Camurati- | BC000125 | 6.24 | 7.34 |
| Engelmann disease) | | | |
| Transforming growth factor, β-induced, 68 kDa | NM_000358 | 9.71 | 11.08 |
| Transglutaminase 2 (C polypeptide, protein- | AL031651 | 7 | 9.55 |
| glutamine-γ-glutamyltransferase) | | | |
| Transmembrane 4 L 6 family member 1 | AI346835 | 9.02 | 11.05 |
| Transmembrane 4 L 6 family member 20 | NM_024795 | 7.12 | 8.72 |
| Transmembrane 4 L 6 family member 4 | BC001386 | 6.7 | 8.65 |
| Transmembrane 7 superfamily member 1 (up- | NM_003272 | 7.21 | 8.32 |
| regulated in kidney) | | | |
| Transmembrane and coiled-coil domains 1 | AI934469 | 6.33 | 7.81 |
| Transmembrane channel-like 5 | BG484769 | 10.71 | 11.78 |
| Transmembrane protein 23 | AI377497 | 7.37 | 8.8 |
| Transmembrane protein 45A | NM_018004 | 6.13 | 8.12 |
| Transmembrane, prostate androgen induced | NM_020182 | 7.16 | 8.82 |
| RNA | | | |
| Transporter 1, ATP-binding cassette, subfamily B | NM_000593 | 8.93 | 10.37 |
| (MDR/TAP) | | | |
| Transporter 2, ATP-binding cassette, subfamily B | AA573502 | 6.68 | 8.21 |
| (MDR/TAP) | | | |
| Transposon-derived Buster1 transposase-like | AA813103 | 5.32 | 6.65 |
| protein gene | | | |
| Trefoil factor 1 (breast cancer, estrogen-inducible | NM_003225 | 11.42 | 13.08 |
| sequence expressed in) | | | |
| Trefoil factor 2 (spasmolytic protein 1) | NM_005423 | 8.29 | 9.4 |
| Tribbles homolog 2 (Drosophila) | NM_021643 | 5.6 | 8.17 |
| Tribbles homolog 3 (Drosophila) | NM_021158 | 6.03 | 7.14 |
| Trichorhinophalangeal syndrome I | T56980 | 6.08 | 7.79 |
| Tripartite motif-containing 22 | AA083478 | 8.76 | 10.32 |
| Tropomyosin 3 /// tropomyosin 4 | AF362887 | 6.07 | 7.66 |
| Tropomyosin 4 | AI214061 | 8.29 | 9.35 |
| Tryptase α/β 1 | NM_003294 | 8.41 | 9.43 |
| Tryptase β 2 | NM_024164 | 8.4 | 9.48 |
| Tryptophan 2,3-dioxygenase | NM_005651 | 5.67 | 8.55 |
| Tryptophanyl-tRNA synthetase | NM_004184 | 8.6 | 10.32 |
| Tubulin, α 3 | AF141347 | 9.31 | 10.42 |
| Tubulin, β 2 | NM_001069 | 8.63 | 10.35 |
| Tubulin, β 6 | BC002654 | 7.91 | 9.26 |
| Tumor necrosis factor (ligand) superfamily, | AF114013 | 7.46 | 8.77 |
| member 13 /// tumor necrosis factor (ligand) | | | |
| superfamily, member 12-member 13 | | | |
| Tumor necrosis factor (ligand) superfamily, | AW151360 | 7.51 | 8.74 |
| member 13b | | | |
| Tumor necrosis factor receptor superfamily, | NM_002546 | 6.64 | 7.67 |
| member 11b (osteoprotegerin) | | | |
| Tumor necrosis factor receptor superfamily, | NM_001066 | 7.69 | 8.79 |
| member 1B | | | |
| Tumor necrosis factor receptor superfamily, | NM_003823 | 7.1 | 9.67 |
| member 6b, decoy /// regulator of telomere | | | |
| elongation helicase 1 | | | |
| Tumor necrosis factor, α-induced protein 2 | NM_006291 | 7.66 | 8.74 |
| Tumor necrosis factor, α-induced protein 6 | NM_007115 | 6.99 | 8.13 |
| Tumor necrosis factor, α-induced protein 8 | NM_014350 | 9.15 | 10.37 |
| Tumor necrosis factor, α-induced protein 9 | AA650281 | 6.23 | 7.8 |
| Tumor protein D52-like 1 | NM_003287 | 6.85 | 8.85 |
| Tumor rejection antigen (gp96) 1 | AK025862 | 8.68 | 9.95 |
| Tumor-associated calcium signal transducer 2 | J04152 | 5.57 | 7.46 |
| Twisted gastrulation homolog 1 (Drosophila) | NM_020648 | 5.86 | 6.93 |
| Tyrosylprotein sulfotransferase 2 | NM_003595 | 7.56 | 8.77 |
| Ubiquitin D | NM_006398 | 7.69 | 10.96 |
| Ubiquitin-conjugating enzyme E2, J2 (UBC6 | BE962920 | 9.02 | 10.28 |
| homolog, yeast) | | | |
| Ubiquitin-conjugating enzyme E2H (UBC8 | AI829920 | 5.82 | 7 |
| homolog, yeast) | | | |
| Ubiquitin-conjugating enzyme E2L 6 | NM_004223 | 9 | 10.15 |
| UDP-N-acetyl-α-d-galactosamine:polypeptide N- | NM_004481 | 7.77 | 8.99 |
| acetylgalactosaminyltransferase 2 (GalNAc-T2) | | | |
| UDP-N-acetyl-α-d-galactosamine:polypeptide N- | NM_007210 | 6.81 | 8.05 |
| acetylgalactosaminyltransferase 6 (GalNAc-T6) | | | |
| Uridine phosphorylase 1 | NM_003364 | 7.57 | 8.72 |
| UTP14, U3 small nucleolar ribonucleoprotein, | NM_006649 | 5.33 | 7.69 |
| homolog A (yeast) | | | |
| Vacuolar protein sorting 13C (yeast) | AV703288 | 7.3 | 8.35 |
| V-akt murine thymoma viral oncogene homolog 3 | U79271 | 6.13 | 7.15 |
| (protein kinase B, γ) | | | |
| Vanin 1 /// vanin 1 | NM_004666 | 5.62 | 10.01 |
| Vascular cell adhesion molecule 1 | NM_001078 | 7.95 | 9.68 |
| Vascular endothelial growth factor | M27281 | 5.84 | 6.98 |
| v-ets erythroblastosis virus E26 oncogene | BC017314 | 5.7 | 7.24 |
| homolog 1 (avian) | | | |
| v-maf musculoaponeurotic fibrosarcoma | NM_012323 | 6.62 | 7.9 |
| oncogene homolog F (avian) | | | |
| von Willebrand factor | NM_000552 | 6.6 | 8.68 |
| V-set and immunoglobulin domain containing 1 | AW085312 | 5.81 | 9.59 |
| v-yes-1 Yamaguchi sarcoma viral-related | NM_002350 | 8.32 | 10.23 |
| oncogene homolog | | | |
| WD repeat domain 44 | NM_019045 | 7.45 | 9.15 |
| Wingless-type MMTV integration site family, | NM_003392 | 7.53 | 9.41 |
| member 5A | | | |
| Wiskott-Aldrich syndrome protein interacting | AI005043 | 6.29 | 7.57 |
| protein | | | |
| WNT1 inducible signaling pathway protein 1 | AI917494 | 5.46 | 6.91 |
| WW domain containing transcription regulator 1 | BF674349 | 8.7 | 9.94 |
| Zinc finger homeobox 1b | AW611486 | 6.8 | 8.25 |
| Zinc finger protein 226 | NM_015919 | 6.42 | 7.58 |
| Zinc finger protein 42 | BI092935 | 4.13 | 6.99 |
| Zinc finger protein 521 | AK021452 | 5.96 | 7.78 |
| Zinc finger protein 555 | NM_152791 | 5.62 | 6.77 |
| Zinc finger protein 650 | AW293453 | 6.69 | 7.75 |
| Zinc finger protein, multitype 2 | NM_012082 | 4.98 | 6.76 |
| Zinc finger protein, subfamily 1A, 1 (Ikaros) | BG540504 | 6.15 | 7.32 |
| Zinc finger, CSL domain containing 2 | AI825858 | 8.1 | 9.12 |

Down-regulated in UC

| | | | |
|---|---|---|---|
| 3-Hydroxy-3-methylglutaryl-Coenzyme A | NM_005518 | 12.03 | 7.99 |
| synthase 2 (mitochondrial) | | | |
| 3-Hydroxybutyrate dehydrogenase (heart, | BF433037 | 8.42 | 7.36 |
| mitochondrial) | | | |
| a disintegrin-like and metalloprotease (reprolysin | AK023795 | 7.31 | 5.57 |
| type) with thrombospondin type 1 motif, 1 | | | |
| A kinase (PRKA) anchor protein 1 | U34074 | 7.89 | 6.87 |
| Acetyl-coenzyme A acetyltransferase 1 | NM_000019 | 10.76 | 9.72 |
| (acetoacetyl coenzyme A thiolase) | | | |
| Activin A receptor, type IC | NM_145259 | 9.28 | 8.17 |
| Acyl-coenzyme A dehydrogenase, C-2 to C-3 | NM_000017 | 8.15 | 6.78 |
| short chain | | | |
| Acyl-coenzyme A oxidase 1, palmitoyl | T62985 | 9.73 | 8.62 |
| Adenosine monophosphate deaminase 1 | NM_000036 | 7.06 | 5.25 |
| (isoform M) | | | |
| Alcohol dehydrogenase 1A (class I), α | NM_000667 | 7.73 | 6.48 |
| polypeptide | | | |
| Alcohol dehydrogenase 1C (class I), γ | NM_000669 | 11.38 | 8.31 |
| polypeptide | | | |
| α-Methylacyl-CoA racemase | AF047020 | 8.56 | 7.41 |
| Amnionless homolog (mouse) | AW051926 | 8.16 | 6.37 |
| Amyotrophic lateral sclerosis 2 (juvenile) | AB038950 | 9.57 | 8.55 |
| chromosome region, candidate 2 | | | |
| Ankyrin 3, node of Ranvier (ankyrin G) | NM_020987 | 9.56 | 8.51 |
| Ankyrin repeat domain 9 | AW138815 | 8.36 | 7.09 |
| APG10 autophagy 10-like (S cerevisiae) | BC018651 | 7.04 | 6.01 |
| Apolipoprotein B mRNA editing enzyme, catalytic | NM_004900 | 8.77 | 6.69 |
| polypeptide-like 3B | | | |
| Aquaporin 8 | NM_001169 | 12.27 | 8.34 |
| Arylsulfatase D | AI741110 | 8.94 | 7.85 |
| ATP-binding cassette, subfamily A (ABC1), | AI568925 | 7.43 | 6.31 |
| member 5 | | | |
| ATP-binding cassette, subfamily A (ABC1), | NM_007168 | 9.1 | 6.96 |
| member 8 | | | |
| ATP-binding cassette, subfamily B (MDR/TAP), | AF016535 | 9.35 | 7.94 |
| member 1 | | | |
| ATP-binding cassette, subfamily C (CFTR/MRP), | BF515888 | 9.44 | 7.94 |
| member 3 | | | |
| ATP-binding cassette, subfamily G (WHITE), | AF098951 | 10.28 | 8.54 |
| member 2 | | | |
| Branched chain aminotransferase 2, | NM_001190 | 8.06 | 6.28 |
| mitochondrial | | | |
| Breast cancer metastasis-suppressor 1-like | AA779684 | 8.27 | 6.99 |
| BTB/POZ-zinc finger protein-like | AI278995 | 9.07 | 7.64 |
| Calcium channel, voltage-dependent, γ subunit 5 | NM_145811 | 7.93 | 5.98 |
| Calcium/calmodulin-dependent protein kinase ID | AA835485 | 9.72 | 8.65 |
| Calcium/calmodulin-dependent protein kinase II | NM_018584 | 11.85 | 10.75 |
| Calpain 13 | AK026692 | 7.31 | 5.12 |
| Carbonic anhydrase VII | NM_005182 | 8.32 | 7.31 |
| Carbonic anhydrase XII | NM_001218 | 11.51 | 10.29 |
| Carboxylesterase 2 (intestine, liver) | D50579 | 10.97 | 9.77 |
| Chemokine-like factor super family 4 | AK000855 | 9.91 | 8.86 |
| Chloride channel 2 | NM_004366 | 8.66 | 7.33 |
| Cholinergic receptor, nicotinic, α polypeptide 1 | NM_000079 | 6.88 | 4.98 |
| (muscle) | | | |
| Chromogranin A (parathyroid secretory protein 1) | NM_001275 | 11.07 | 9.53 |
| Churchill domain containing 1 | BE568660 | 8.4 | 7.38 |
| Citrate lyase β like | BG398847 | 7.84 | 6.62 |
| Claudin 8 | AL049977 | 10.88 | 7.32 |
| Coenzyme Q6 homolog (yeast) | AA528157 | 11.27 | 9.78 |
| Collomin | AW006648 | 8.07 | 6.5 |
| Contactin 3 (plasmacytoma associated) | BE221817 | 7.61 | 5.49 |
| Creatine kinase, brain | NM_001823 | 12.36 | 11.23 |
| CTD (carboxy-terminal domain, RNA polymerase | NM_005808 | 9.26 | 8.23 |
| II, polypeptide A) small phosphatase-like | | | |
| Cyclin-dependent kinase (CDC2-like) 10 | AF153430 | 6.7 | 5.67 |
| Cyclin-dependent kinase inhibitor 2B (p15, | AW444761 | 9.79 | 8.17 |
| inhibits CDK4) | | | |
| Cystathionine-β-synthase | BC007257 | 7.29 | 6.1 |
| Cystatin SA | NM_001322 | 7.9 | 6.22 |
| Cytochrome c, somatic | AI760495 | 7.55 | 6.08 |
| Cytochrome P450, family 2, subfamily B, | NM_000767 | 7.58 | 6.13 |
| polypeptide 7 pseudogene 1 | | | |
| Cytochrome P450, family 2, subfamily J, | NM_000775 | 8.47 | 7.3 |
| polypeptide 2 | | | |
| Cytochrome P450, family 4, subfamily F, | NM_023944 | 9.29 | 7.77 |
| polypeptide 12 | | | |
| Dedicator of cytokinesis 1 | AK000789 | 8.25 | 7.02 |
| Defensin, β 1 | U73945 | 8.69 | 7.26 |
| DEP domain containing 6 | NM_022783 | 8.07 | 7.05 |
| Dimethylarginine dimethylaminohydrolase 2 | NM_013974 | 8.37 | 7.22 |
| Dimethylarginine dimethylaminohydrolase 2 | AJ012008 | 8.39 | 7.2 |
| dopa decarboxylase (aromatic I-amino acid | NM_000790 | 8.2 | 6.77 |
| decarboxylase) | | | |
| Early growth response 1 | AI459194 | 9.98 | 8.91 |
| Ectonucleoside triphosphate diphosphohydrolase 5 | NM_001249 | 9.84 | 8.23 |
| Enoyl coenzyme A hydratase 1, peroxisomal | NM_001398 | 10.08 | 8.68 |
| Enoyl coenzyme A hydratase domain containing 2 | AI903313 | 8.1 | 7.01 |
| Ephrin-A1 | NM_004428 | 10.6 | 9.3 |
| Epoxide hydrolase 2, cytoplasmic | AF233336 | 8.18 | 6.68 |
| Erythrocyte membrane protein band 4.1 like 4B | NM_019114 | 11.26 | 10.16 |
| Esterase 31 | NM_024922 | 9.37 | 8.04 |
| ets variant gene 6 (TEL oncogene) | BF436898 | 7.52 | 6.3 |
| Family with sequence similarity 55, member A | NM_152315 | 10.52 | 9.4 |
| Fatty acid amide hydrolase | NM_001441 | 8.3 | 6.43 |
| Fc fragment of IgG, receptor, transporter, α | NM_004107 | 10.4 | 9.33 |
| Fibroblast growth factor 9 (glia-activating factor) | NM_002010 | 7.28 | 5.86 |
| Fibroblast growth factor receptor 2 (bacteria- | NM_022969 | 8.87 | 7.77 |
| expressed kinase, keratinocyte growth factor | | | |
| receptor, craniofacial dysostosis 1, Crouzon | | | |
| syndrome, Pfeiffer syndrome, Jackson-Weiss | | | |
| syndrome) | | | |
| Fibroblast growth factor receptor 3 | NM_000142 | 8.71 | 6.87 |
| (achondroplasia, thanatophoric dwarfism) | | | |
| Flavin-containing mono-oxygenase 5 | AK022172 | 7.02 | 5.98 |
| Forkhead box A2 | AB028021 | 9.1 | 8.01 |
| Frizzled-related protein | NM_001463 | 8.26 | 7.18 |
| FXYD domain containing ion transport regulator 3 | NM_005971 | 11.67 | 10.2 |
| G-protein-coupled receptor 125 | M37712 | 9.06 | 7.67 |
| γ-Aminobutyric acid (GABA) A receptor, α 2 | NM_000807 | 7.22 | 5.82 |
| GATA-binding protein 6 | BF002339 | 8.93 | 7.81 |
| Glucosaminyl (N-acetyl) transferase 2, I- | BF059748 | 7.49 | 5.19 |
| branching enzyme | | | |
| Glucosidase, β, acid 3 (cytosolic) | AW235567 | 7.78 | 6.55 |
| Glutathione S-transferase M4 | NM_000850 | 7.21 | 5.66 |
| G-rich RNA sequence binding factor 1 | BF058465 | 7.17 | 6.1 |
| Growth hormone receptor | NM_000163 | 7.2 | 5.89 |
| Guanylate cyclase activator 2A (guanylin) | NM_002098 | 12.62 | 10.96 |
| Hepatocellular carcinoma antigen gene 520 | NM_022097 | 11.05 | 8.2 |
| HERV-H LTR-associating 2 | AK027132 | 6.8 | 5.68 |
| Homeo box A5 | NM_019102 | 8.66 | 7.48 |
| Homeo box A7 | AF026397 | 7.31 | 6.29 |
| Homeo box D10 | AW299531 | 7.42 | 5.88 |
| Hydroxysteroid (11-β) dehydrogenase 2 | NM_000196 | 10.96 | 9.36 |
| InaD-like (Drosophila) | AJ001306 | 7.88 | 6.77 |
| Inositol 1,4,5-trisphosphate 3-kinase A | NM_002220 | 8.73 | 7.45 |
| Inositol hexaphosphate kinase 2 | BC004469 | 9.06 | 7.91 |
| IL-1 receptor, type II | NM_004633 | 10.94 | 9.49 |
| IL-11 receptor, α | NM_004512 | 6.68 | 5.57 |
| Itchy homolog E3 ubiquitin protein ligase (mouse) | AA868238 | 7.69 | 6.57 |
| Kallikrein 1, renal/pancreas/salivary | L10038 | 10.45 | 8.26 |
| Ku70-binding protein 3 | AI628122 | 8.78 | 7.73 |
| Laminin, α 1 | AI990816 | 9.36 | 7.74 |
| Lectin, galactoside-binding, soluble, 2 (galectin 2) | NM_006498 | 9.98 | 7.92 |
| Lethal giant larvae homolog 2 (Drosophila) | AF289551 | 7.33 | 6.28 |
| Leukotriene B4 12-hydroxydehydrogenase | BE566894 | 8.11 | 6.79 |
| LY6/PLAUR domain containing 1 | AW268162 | 7.22 | 5.88 |
| maba1 | AB037745 | 8.91 | 7.73 |
| Macrophage stimulating 1 (hepatocyte growth | U37055 | 7.77 | 6.35 |
| factor-like) | | | |
| Macrophage stimulating, pseudogene 9 | U28055 | 7.23 | 5.05 |
| MAD1 mitotic arrest deficient-like 1 (yeast) | AK022078 | 6.93 | 5.31 |
| MAM domain containing 2 | AI862120 | 7.29 | 5.99 |
| Matrix metalloproteinase 28 | NM_024302 | 7.33 | 6.33 |
| MAWD binding protein | NM_022129 | 9.82 | 8.57 |
| Membrane progestin receptor γ | AI934557 | 9.72 | 8 |
| Membrane-spanning 4-domains, subfamily A, | NM_017716 | 12.34 | 11.18 |
| member 12 | | | |
| Meprin A, α (PABA peptide hydrolase) | NM_005588 | 11.25 | 10.06 |
| Meprin A, β | NM_005925 | 6.83 | 5.47 |
| Mercaptopyruvate sulfurtransferase | NM_021126 | 9.64 | 8.29 |
| Metallothionein 1E (functional) | BF217861 | 12.46 | 11.33 |
| Metallothionein 1F (functional) | BF246115 | 12.18 | 10.58 |
| Metallothionein 1G | NM_005950 | 12.44 | 10.9 |
| Metallothionein 1H | NM_005951 | 12.06 | 10.48 |
| Metallothionein 1K | AL031602 | 11.31 | 10.17 |
| Metallothionein 1X | NM_002450 | 12.02 | 10.72 |
| Metallothionein 2A | NM_005953 | 13.03 | 11.98 |
| Mitochondrial ribosomal protein S25 | AK024433 | 6.69 | 5.53 |
| Mitogen-activated protein kinase kinase kinase | BF739979 | 8.9 | 7.81 |
| 15 | | | |
| Monoacylglycerol O-acyltransferase 2 | AK000245 | 9.33 | 8.29 |
| Monoamine oxidase A | AA923354 | 11.69 | 10.46 |
| Mucin 12 | AF147790 | 11.17 | 10.05 |
| Mucin 20 | AB037780 | 8.16 | 7.02 |
| Mucolipin 2 | AV713773 | 9.28 | 7.74 |
| Myofibrillogenesis regulator 1 | AA074597 | 8.6 | 7.6 |
| Myosin IA /// myosin IA | AF009961 | 9.17 | 8.09 |
| Myosin VIIB | AK000145 | 6.87 | 5.54 |
| Myotubularin related protein 11 | NM_006697 | 10.3 | 8.85 |
| Myristoylated alanine-rich protein kinase C | AW163148 | 9.02 | 8 |
| substrate | | | |
| N-acylsphingosine amidohydrolase (acid | AK025371 | 7.7 | 6 |
| ceramidase)-like | | | |
| NADH dehydrogenase (ubiquinone) 1 β | AF044954 | 10.22 | 9.18 |
| subcomplex, 10, 22 kDa | | | |
| NADH dehydrogenase (ubiquinone) Fe—S protein | AI808395 | 7.8 | 6.63 |
| 1, 75 kDa (NADH-coenzyme Q reductase) | | | |
| NADH dehydrogenase (ubiquinone) flavoprotein | AF092131 | 10.72 | 9.67 |
| 1, 51 kDa | | | |
| Neural precursor cell expressed, developmentally | AL833742 | 7.86 | 5.81 |
| down-regulated 4-like | | | |
| Neuron navigator 1 | AB033039 | 7.62 | 6.32 |
| Neuronal guanine nucleotide exchange factor | AV703769 | 8.29 | 7.24 |
| Neuropeptide Y | NM_000905 | 6.93 | 5.57 |
| NOL1/NOP2/Sun domain family, member 5 | NM_018044 | 9.35 | 8.05 |
| NS5ATP13TP2 protein | BG033561 | 9.38 | 7.74 |
| Nuclear pore complex interacting protein /// | AC002045 | 10.49 | 9.44 |
| hypothetical protein LOC339047 /// similar to | | | |
| hypothetical protein LOC339047 | | | |
| Nuclear receptor subfamily 1, group D, member 2 | AI761621 | 8.09 | 6.88 |
| OGT(O-Glc-NAc transferase)-interacting protein | J03068 | 7.21 | 5.54 |
| 106 kDa | | | |
| Olfactory receptor, family 2, subfamily H, | AJ459849 | 7.44 | 6.24 |
| member 1 | | | |
| Organic solute transporter α | AA702685 | 7.61 | 6.09 |
| ORM1-like 1 (S cerevisiae) | AI923278 | 6.8 | 5.75 |
| Oxysterol binding protein-like 7 | AI955239 | 8.71 | 7.68 |
| Paired immunoglobulin-like type 2 receptor β | NM_013440 | 7.6 | 6.49 |
| Pancreatic lipase-related protein 2 | BC005989 | 8.11 | 4.94 |
| PDZ domain containing 1 | NM_002614 | 7.98 | 5.45 |
| PDZ domain containing 2 | NM_024791 | 7.37 | 5.94 |
| Peptide YY | NM_004160 | 7.64 | 5.77 |
| per1-like domain containing 1 | BF033007 | 7.45 | 6.2 |
| Period homolog 3 (Drosophila) | NM_016831 | 7.1 | 5.72 |
| Peroxiredoxin 6 | NM_004905 | 12.24 | 11.24 |
| Peroxisomal membrane protein 2, 22 kDa | NM_018663 | 9.52 | 8.16 |
| Peroxisome proliferative activated receptor, γ, | NM_013261 | 8.67 | 7.19 |
| coactivator 1, α | | | |
| Phosphatase and actin regulator 4 | NM_023923 | 6.82 | 5.77 |
| Phosphodiesterase 6A, cGMP-specific, rod, α | NM_000440 | 6.78 | 5.1 |
| Phosphodiesterase 9A | NM_002606 | 10.45 | 8.68 |
| Phosphoenolpyruvate carboxykinase 1 (soluble) | NM_002591 | 11.67 | 8.96 |
| Phospholipase C, epsilon 1 | NM_016341 | 8.2 | 7.16 |
| Phosphomannomutase 1 | NM_002676 | 8.89 | 7.62 |
| Phosphorylase kinase, α 2 (liver) | D38616 | 7.34 | 5.89 |
| Pleckstrin and Sec7 domain containing 3 | AW117368 | 8.78 | 7.67 |
| Pleckstrin homology domain containing, family A | AA535361 | 8.04 | 6.91 |
| member 6 | | | |
| Pleiotrophin (heparin binding growth factor 8, | AL565812 | 7.89 | 6.21 |
| neurite growth-promoting factor 1) | | | |
| Plexin A2 | AI688418 | 8.37 | 7.11 |
| PLSC domain containing protein | BG255923 | 6.98 | 5.89 |
| Polycythemia rubra vera 1 | NM_020406 | 11.69 | 9.78 |
| Prion protein interacting protein | BC001072 | 7.89 | 6.76 |
| Programmed cell death 4 (neoplastic | NM_014456 | 9.73 | 8.54 |
| transformation inhibitor) | | | |
| Prolactin receptor | AA843963 | 8.02 | 6.74 |
| Proline-rich acidic protein 1 | AA502331 | 9.78 | 8.67 |
| Proline-rich protein PRP2 | NM_173490 | 8.8 | 7.71 |
| Prominin 2 | NM_144707 | 8.99 | 7.71 |
| Prostaglandin D2 receptor (DP) | AI460323 | 8.82 | 7.32 |
| Protein tyrosine phosphatase, receptor type, O | NM_002848 | 8.29 | 7.11 |
| Protocadherin 21 | AI825832 | 8.79 | 5.69 |
| Queuine tRNA-ribosyltransferase 1 (tRNA- | NM_031209 | 7.72 | 6.39 |
| guanine transglycosylase) | | | |
| RAB40B, member RAS oncogene family | NM_006822 | 8.31 | 6.92 |
| Rap guanine nucleotide exchange factor (GEF)- | NM_016339 | 8.4 | 6.73 |
| like 1 | | | |
| Rap2-binding protein 9 | AI928037 | 8.01 | 6.73 |
| Ras responsive element binding protein 1 | BF591556 | 10.04 | 9.02 |
| RAX-like homeobox | AK025181 | 10.1 | 7.51 |
| Ribonuclease, RNase A family, 1 (pancreatic) | NM_002933 | 10.46 | 9.44 |
| Ring finger protein 157 | BF056204 | 7.04 | 5.95 |
| SA hypertension-associated homolog (rat) | D16350 | 7.58 | 6.52 |
| SATB family member 2 | AB028957 | 10.63 | 9.39 |
| Selenium-binding protein 1 | NM_003944 | 12.51 | 10.08 |
| sema domain, transmembrane domain (TM), and | AB002438 | 9.13 | 8.04 |
| cytoplasmic domain, (semaphorin) 6A | | | |
| Serine palmitoyltransferase, long chain base | H68862 | 7.85 | 6.76 |
| subunit 2 | | | |
| Serine protease inhibitor, Kazal type 2 (acrosin- | NM_021114 | 7.1 | 5.41 |
| trypsin inhibitor) | | | |
| Serum/glucocorticoid regulated kinase 2 | AI631895 | 8.4 | 6.32 |
| SH3 domain containing ring finger 2 | AW082633 | 8.63 | 7.46 |
| Short-chain dehydrogenase/reductase | NM_024308 | 9.86 | 8.21 |
| SLAC2-B | AB014524 | 10.23 | 8.71 |
| Small EDRK-rich factor 1A (telomeric) | AF073518 | 9.16 | 8.11 |
| Small nuclear protein PRAC | BG498699 | 11.32 | 10.03 |
| Sodium channel, nonvoltage-gated 1, β (Liddle | NM_000336 | 9.13 | 7.31 |
| syndrome) | | | |
| Solute carrier family 16 (monocarboxylic acid | NM_003051 | 10.55 | 8.52 |
| transporters), member 1 | | | |
| Solute carrier family 16 (monocarboxylic acid | BG401568 | 10.2 | 8.44 |
| transporters), member 9 | | | |
| Solute carrier family 22 (organic cation | NM_003060 | 9.88 | 8.73 |
| transporter), member 5 | | | |
| Solute carrier family 26 (sulfate transporter), | AI025519 | 13.29 | 10.97 |
| member 2 | | | |
| Solute carrier family 26, member 3 | AK025044 | 10.38 | 8.97 |
| Solute carrier family 30 (zinc transporter), | NM_018713 | 7.27 | 6.23 |
| member 10 | | | |
| Solute carrier family 36 (proton/amino acid | AW058600 | 9.76 | 8.09 |
| symporter), member 1 | | | |
| Solute carrier family 38, member 4 | NM_018018 | 6.84 | 4.82 |
| Solute carrier family 4, sodium bicarbonate | NM_003759 | 11.64 | 10.5 |
| cotransporter, member 4 | | | |
| Solute carrier family 6 (neutral amino acid | AI627358 | 7.51 | 5.42 |
| transporter), member 19 | | | |
| Solute carrier family 9 (sodium/hydrogen | AF073299 | 7.14 | 5.41 |
| exchanger), isoform 2 | | | |
| Sorcin | AV752215 | 10.37 | 9.27 |
| Spectrin, α, nonerythrocytic 1 (α-fodrin) | AK026484 | 7.91 | 6.69 |
| Sphingomyelin phosphodiesterase, acid-like 3B | NM_014474 | 7.3 | 5 |
| Spire homolog 2 (Drosophila) | BC011119 | 7.68 | 6.58 |
| ST6 (α-N-acetyl-neuraminyl-2,3-β-galactosyl- | AK023900 | 10.56 | 9.44 |
| 1,3)-N-acetylgalactosaminide α-2,6- | | | |
| sialyltransferase 6 | | | |
| START domain containing 10 | AF151810 | 10.57 | 8.98 |
| Succinate dehydrogenase complex, subunit A, | AW090199 | 8.97 | 7.82 |
| flavoprotein-like 2 | | | |
| sulfotransferase family, cytosolic, 1A, phenol- | U37025 | 10.83 | 9.38 |
| preferring, member 1 | | | |
| sulfotransferase family, cytosolic, 1A, phenol- | U28169 | 10.27 | 9.21 |
| preferring, member 2 | | | |
| Testis expressed sequence 11 | AL139109 | 7.55 | 4.64 |
| Tetraspanin 7 | NM_004615 | 10.41 | 8.78 |
| Thyrotrophic embryonic factor | AA779795 | 7.55 | 6.04 |
| Transcription elongation factor A (SII), 3 | AI675780 | 10.37 | 9.12 |
| Transcription factor CP2-like 1 | AI632567 | 7.78 | 6.26 |
| Transient receptor potential cation channel, | NM_017636 | 10.24 | 8.82 |
| subfamily M, member 4 | | | |
| Transient receptor potential cation channel, | BF447669 | 9.41 | 7.42 |
| subfamily M, member 6 | | | |
| Transmembrane emp24 protein transport domain | AA708152 | 6.98 | 5.85 |
| containing 6 | | | |
| Transmembrane protein 20 | AI452512 | 6.97 | 5.59 |
| Transmembrane protein 38B | NM_018112 | 8.32 | 7.2 |
| Triggering receptor expressed on myeloid cells 5 | BC028091 | 6.66 | 5.58 |
| Tryptophan hydroxylase 1 (tryptophan 5-mono- | AI350339 | 8.47 | 6.92 |
| oxygenase) | | | |
| Tubulin, α-like 3 | NM_024803 | 8.85 | 7.44 |
| Tumor protein p53 inducible protein 5 | AW084755 | 8.8 | 7.79 |
| Ubiquitin-specific protease 2 | AW274034 | 7.17 | 5.64 |
| Ubiquitin-specific protease 30 | BC004868 | 6.81 | 5.76 |
| UDP glycosyltransferase 1 family, polypeptide A3 | NM_019093 | 9.72 | 7.97 |
| UDP glycosyltransferase 1 family, polypeptide A6 | NM_001072 | 10.41 | 8.66 |
| UDP glycosyltransferase 1 family, polypeptide A8 | NM_019076 | 8.24 | 6.01 |
| UDP-Gal:β-Gal β 1,3-galactosyltransferase | AI760623 | 7.75 | 6.65 |
| polypeptide 7 | | | |
| UDP-GlcNAc:β-Gal β-1,3-N- | CA503291 | 10.69 | 9.05 |
| acetylglucosaminyltransferase 7 | | | |
| UDP-N-acetyl-α-d-galactosamine:polypeptide N- | NM_024642 | 10.21 | 9.07 |
| acetylgalactosaminyltransferase 12 (GalNAc-T12) | | | |
| Vasoactive intestinal peptide receptor 1 | NM_004624 | 8.91 | 7.61 |
| vav 3 oncogene | NM_006113 | 10.34 | 9.25 |
| V-erb-a erythroblastic leukemia viral oncogene | R48991 | 7.88 | 6.63 |
| homolog 4 (avian) | | | |
| Villin 1 | NM_007127 | 9.91 | 8.89 |
| Vitelliform macular dystrophy 2-like 1 | NM_017682 | 9.18 | 6.44 |
| Vitelliform macular dystrophy 2-like 2 | NM_153274 | 7.98 | 6.69 |
| v-ral simian leukemia viral oncogene homolog B | BG169673 | 10.94 | 9.88 |
| (ras related; GTP binding protein) | | | |
| WAP 4-disulfide core domain 2 | NM_006103 | 10.21 | 8.54 |
| WD repeat domain 9 | AW268572 | 8.58 | 7.52 |
| Williams Beuren syndrome chromosome region | N29665 | 9.16 | 8.11 |
| 20C | | | |
| WNK lysine deficient protein kinase 2 | AI637586 | 8.72 | 7.39 |
| WNK lysine deficient protein kinase 4 | AW082836 | 7.77 | 5.8 |
| Zinc finger and BTB domain containing 33 | BG391005 | 6.86 | 3.27 |
| Zinc finger protein 291 | AK025663 | 7.45 | 6.32 |
| Zinc finger protein 395 | NM_018660 | 7.46 | 6.41 |
| Zinc finger with KRAB and SCAN domains 1 | BG761185 | 8.14 | 7.04 |

TABLE 9
miRNAs Differentially Expressed in Crohn's Disease
(CD) Tissues as Compared With Normal, Healthy Controls

| microRNA | Normal | Crohn's | Fold Change |
|---|---|---|---|
| miR-23b | 0.0684 | 0.1132 | +1.66 |
| miR-192 | 1.652 | 0.687 | −2.40 |
| Let-7b | 0.7197 | 0.2649 | −2.72 |
| Let-7a | 0.5493 | 0.3025 | −1.82 |
| miR-26a | 0.4415 | 0.2775 | −1.59 |
| miR-19b | 0.3790 | 0.1373 | −2.76 |
| Let-7f | 0.2600 | 0.1458 | −1.78 |
| miR-126 | 0.2384 | 0.1480 | −1.61 |
| miR-320 | 0.1326 | 0.0439 | −3.02 |
| miR-203 | 0.0428 | 0.0185 | −2.31 |
| miR-422b (also known as miR-378) | 0.0346 | 0.0191 | −1.81 |
| miR-629 | 0.0225 | 0.0063 | −3.57 |
| miR-29a | 0.0221 | 0.0112 | −1.97 |

TABLE 10
miRNAs Differentially Expressed in Crohn's Disease (CD) Terminal
Ileal Biopsy Tissues as Compared With Normal, Healthy Controls
microRNA

miR-422b (also known as miR-378)
miR-16
miR-21
miR-594
Let-7i
miR-20a
miR-223

TABLE 11
miRNAs Differentially Expressed in Blood Samples From
Subjects Having Active Ulcerative Colitis (UC) as
Compared With Normal, Healthy Controls
microRNA

miR-574
miR-516
miR-300
miR-1275

TABLE 12
miRNAs Differentially Expressed in Blood Samples From
Subjects Having Active Crohn's Disease (CD) as
Compared With Normal, Healthy Controls
microRNA

miR-939
miR-765
miR-628-3p
miR-583
miR-574-5p
miR-516a-5p
miR-32*
miR-300
miR-193b*
miR-1299
miR-125b-1*
miR-1184

TABLE 13
miRNAs Differentially Expressed in Blood Samples From
Subjects Having TNBS- Induced Ulcerative Colitis (UC)
as Compared With Normal, Healthy Controls

| microRNA upregulated | MicroRNA downregulated |
|---|---|
| miR-34b | miR-375 |
| miR-679 | miR-26a |
| miR-449b | miR-23a |
| miR-21 | miR-23b |
| | miR-200c |
| | miR-145 |
| | miR-143 |
| | miR-133b |
| | miR-133a |
| | miR-200a |
| | miR-30c |
| | Let-7b |
| | Let-7c |
| | miR-422b (also known as miRNA-378) |
| | miR-195 |
| | miR-24 |
| | miR-194 |
| | miR-103 |
| | miR-30d |
| | Let-7a |
| | Let-7d |
| | miR-107 |
| | miR-27b |

TABLE 14
Sequences of miRNAs described in Tables 1-13

| microRNA | Mature microRNA sequence (5′-3′) |
|---|---|
| Let-7a | ugagguaguagguuguauaguu |
| Let-7b | ugagguaguagguugugugguu |
| Let-7f | ugagguaguagauuguauaguu |
| Let-7i | ugagguaguaguuugugcuguu |
| miR-106a | aaaagugcuuacagugcagguag |
| miR-107 | agcagcauuguacagggcuauca |
| miR-1184 | ccugcagcgacuugauggcuucc |
| miR-125b-1* | acggguuaggcucuugggagcu |
| miR-126 | ucguaccgugaguaauaaugcg |
| miR-1275 | gugggggagaggcuguc |
| miR-1299 | uucuggaauucugugugaggga |
| miR-141 | uaacacugucugguaaagaugg |
| miR-150 | ucucccaacccuuguaccagug |
| miR-16 | uagcagcacguaaauauuggcg |
| miR-191 | caacggaaucccaaaagcagcug |
| miR-192 | cugaccuaugaauugacagcc |
| miR-193b | aacuggcccucaaagucccgcu |
| miR-195 | uagcagcacagaaauauuggc |
| miR-199a | cccaguguucagacuaccuguuc |
| miR-19b | ugugcaaauccaugcaaaacuga |
| miR-200a | uaacacugucugguaacgaugu |
| miR-200b | uaauacugccugguaaugauga |
| miR-200c | uaauacugccggguaaugaugga |
| miR-203 | gugaaauguuuaggaccacuag |
| miR-20a | uaaagugcuuauagugcagguag |
| miR-21 | uagcuuaucagacugauguuga |
| miR-215 | augaccuaugaauugacagac |
| miR-217 | uacugcaucaggaacugauugga |
| miR-223 | ugucaguuugucaaauacccca |
| miR-23a | aucacauugccagggauuucc |
| miR-23b | aucacauugccagggauuacc |
| miR-24 | uggcucaguucagcaggaacag |
| miR-26a | uucaaguaauccaggauaggcu |
| miR-27b | uucacaguggcuaaguucugc |
| miR-29a | uagcaccaucugaaaucgguua |
| miR-300 | uauacaagggcagacucucucu |
| miR-32* | caauuuagugugugugauauuu |
| miR-320 | aaaagcuggguugagagggcga |
| miR-375 | uuuguucguucggcucgcguga |
| miR-422b | acuggacuuggagucagaagg |
| (also known as | |
| miRNA-378) | |
| miR-516a-5p | uucucgaggaaagaagcacuuuc |
| miR-532 | caugccuugaguguaggaccgu |
| miR-574-5p | ugagugugugugugugagugugu |
| miR-583 | caaagaggaaggucccauuac |
| miR-603 | cacacacugcaauuacuuuugc |
| miR-628-3p | ucuaguaagaguggcagucga |
| miR-629 | uggguuuacguugggagaacu |
| miR-765 | uggaggagaaggaaggugaug |
| miR-769-5p | ugagaccucuggguucugagcu |
| miR-939 | uggggagcugaggcucugggggug |

1. A method of determining whether a subject is afflicted with an inflammatory bowel disease, condition, or subtype thereof, the method comprising:
a) determining the level of expression or activity of a biomarker listed in Tables 2-14 or a fragment thereof in a subject sample;
b) determining the normal level of expression or activity of the biomarker in a control sample; and
c) comparing the level of expression or activity of said biomarker detected in steps a) and b);
wherein a significant modulation in the level of expression or activity of the biomarker in the subject sample relative to the normal level of expression or activity of the biomarker in a control sample is an indication that the subject is afflicted with an inflammatory bowel disease, condition, or a subtype thereof.

2. The method of claim 1, wherein the inflammatory bowel disease, condition, or subtype thereof is selected from the group consisting of active ulcerative colitis, inactive ulcerative colitis, Crohn's disease, irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis, pseudomembranous colitis, ischemic inflammatory bowel disease, Behcet's disease, sarcoidosis, scleroderma, IBD dysplasia, and dysplasia associated masses or lesions.

3. The method of claim 1, wherein the sample comprises cells, tissue, blood, plasma, serum, stool, or mucus, obtained from the subject.

4. The method of claim 3, wherein the subject cells are obtained from the group consisting of stomach tissue, small intestine tissue, colon tissue, and peripheral blood cell subtypes.

5. The method of claim 1, wherein the expression level of the biomarker is assessed by detecting the presence in the samples of a polynucleotide molecule encoding the biomarker or a portion of said polynucleotide molecule.

6. The method of claim 5, wherein the polynucleotide molecule is a mRNA, cDNA, miRNA, or functional variants or fragments thereof.

7. The method of claim 6, wherein the miRNA or functional variants thereof comprise mature miRNA, pre-miRNA, pri-miRNA, miRNA*, anti-miRNA, or a miRNA binding site.

8. The method of claim 5, wherein the step of detecting further comprises amplifying the polynucleotide molecule.

9. The method of claim 5, wherein the expression level of the biomarker is assessed by annealing a nucleic acid probe with the sample of the polynucleotide encoding the biomarker or a portion of said polynucleotide molecule under stringent hybridization conditions.

10. The method of claim 1, wherein the expression level of the biomarker is assessed by detecting the presence in the samples of a protein of the biomarker, a polypeptide, or protein fragment thereof comprising said protein.

11. The method of claim 10, wherein the presence of said protein, polypeptide or protein fragment thereof is detected using a reagent which specifically binds with said protein, polypeptide or protein fragment thereof.

12. The method of claim 11, wherein the reagent is selected from the group consisting of an antibody, an antibody derivative, and an antibody fragment.

13. The method of claim 1, wherein the activity level of the biomarker is assessed by determining the magnitude of modulation of the activity or expression level of downstream targets of the biomarker.

14. The method of claim 1, wherein said significant modulation comprises an at least two fold increase or an at least two fold decrease between the expression or activity level of the biomarker in the subject sample relative to the normal expression or activity of the biomarker in the sample from the control subject.

15. A method for monitoring the progression of an inflammatory bowel disease, condition, or a subtype thereof in a subject, the method comprising:
a) detecting in a subject sample at a first point in time the level of expression or activity of a biomarker listed in Tables 2-14 or a fragment thereof;
b) repeating step a) at a subsequent point in time; and
c) comparing the level of expression or activity of said biomarker detected in steps a) and b) to monitor the progression of the inflammatory bowel disease, condition, or subtype thereof.

16-18. (canceled)

19. A method for predicting the clinical outcome of a patient, the method comprising:
a) assessing the level of expression or activity of a biomarker listed in Tables 2-14 or a fragment thereof in a patient sample;
b) assessing the level of expression or activity of the biomarker in a sample from a control subject having a good clinical outcome; and
c) comparing the level of expression or activity of the biomarker in the patient sample and in the sample from the control subject;
wherein a significantly modulated level of expression or activity in the patient sample as compared to the expression or activity level in the sample from the control subject predicts the clinical outcome of the patient.

20-24. (canceled)

25. A method of determining the efficacy of a therapy for inhibiting an inflammatory bowel disease, condition, or subtype thereof in a subject, the method comprising comparing:
a) the level of expression or activity of a biomarker listed in Tables 2-14 or a fragment thereof in a first sample obtained from the subject prior to providing at least a portion of the therapy to the subject, and
b) the level of expression or activity of the biomarker in a second sample obtained from the subject following provision of the portion of the therapy,
wherein a significantly modulated level of expression or activity of the biomarker in the second sample, relative to the first sample, is an indication that the therapy is efficacious for inhibiting the inflammatory bowel disease, condition, or subtype thereof in the subject.

26. (canceled)

27. A method for identifying a compound which inhibits an inflammatory bowel disease, condition, or subtype thereof, the method comprising:
a) contacting a biomarker listed in Tables 2-14 or a fragment thereof with a test compound; and
b) determining the effect of the test compound on the level of expression or activity of the biomarker to thereby identify a compound which inhibits an inflammatory bowel disease, condition, or subtype thereof.

28-31. (canceled)

32. A method for inhibiting an inflammatory bowel disease, condition, or subtype thereof, the method comprising contacting a cell with an agent that modulates the expression or activity level of a biomarker listed in Tables 2-14 or a fragment thereof to thereby inhibit an inflammatory bowel disease, condition, or subtype thereof.

33-36. (canceled)

37. A method for treating a subject having an inflammatory bowel disease, condition, or subtype thereof, the method comprising administering an agent that modulates the level of expression or activity of a biomarker listed in Tables 2-14 or a fragment thereof such that the inflammatory bowel disease, condition, or subtype thereof is treated.

38-48. (canceled)
